SEQUENCE LISTING (42 sequences)

SEQ ID NO: 1 - protein, 393 aa, Homo sapiens
MWWRVLSLLAWFPLQEASLTNHTETITVEEGQTLTLKCVTSLRKNSSLQWLTPSGFTIFLNEYPALKNSKYQLLHHSANQLSITVPNVTLQDEGVYKCLHYSDSVSTKEVKVIVLATPFKPILEASVIRKQNGEEHVVLMCSTMRSKPPPQITWLLGNSMEVSGGTLHEFETDGKKCNTTSTLIIHTYGKNSTVDCIIRHRGLQGRKLVAPFRFEDLVTDEETASDALERNSLSSQDPQQPTSTVSVTEDSSTSEIDKEEKEQTTQDPDLTTEANPQYLGLARKKSGILLLTLVSFLIFILFIIVQLFIMKLRKAHVIWKRENEVSEHTLESYRSRSNNEETSSEEKNGQSSHPMRCMNYITKLYSEAKTKRKENVQHSKLEEKHIQVPESIV

SEQ ID NO: 2 - protein, 374 aa, Homo sapiens
TNHTETITVEEGQTLTLKCVTSLRKNSSLQWLTPSGFTIFLNEYPALKNSKYQLLHHSANQLSITVPNVTLQDEGVYKCLHYSDSVSTKEVKVIVLATPFKPILEASVIRKQNGEEHVVLMCSTMRSKPPPQITWLLGNSMEVSGGTLHEFETDGKKCNTTSTLIIHTYGKNSTVDCIIRHRGLQGRKLVAPFRFEDLVTDEETASDALERNSLSSQDPQQPTSTVSVTEDSSTSEIDKEEKEQTTQDPDLTTEANPQYLGLARKKSGILLLTLVSFLIFILFIIVQLFIMKLRKAHVIWKRENEVSEHTLESYRSRSNNEETSSEEKNGQSSHPMRCMNYITKLYSEAKTKRKENVQHSKLEEKHIQVPESIV

SEQ ID NO: 3 - protein, 388 aa, Mus musculus
MWWGVLSLLFWVPVQAAFLKMETVTVEEGQTLTLTCVTSQTKNVSLQWLAPSGFTIFLNQHPALKSSKYQLLHHSATQLSISVSNVTLREEGVYTCLHYGSSVKTKQVRVTVLVTPFQPTVEALVLRRQNGEKSVVLKCSTERSKPPPQITWLLGEGLEIYGELHHEFEADGKICNTSSTLIARAYGKNSTVHCIIQHEGLHGRKLVAPFQFEDLVADQETTSDAPEQSSLSSQALQQPTSTVSMMENSSIPETDKEEKEHATQDPGLSTASAQHTGLARRKSGILLLTLVSFLIFILFIIVQLFIMKLRKAHVVWKKESEISEQALESYRSRSNNEETSSQENSSQAPQSKRCMNYITQLYSGAKTKKSAQHWKLGGKHSRVPESIV

SEQ ID NO: 4 - protein, 372 aa, Mus musculus
AFLKMETVTVEEGQTLTLTCVTSQTKNVSLQWLAPSGFTIFLNQHPALKSSKYQLLHHSATQLSISVSNVTLREEGVYTCLHYGSSVKTKQVRVTVLVTPFQPTVEALVLRRQNGEKSVVLKCSTERSKPPPQITWLLGEGLEIYGELHHEFEADGKICNTSSTLIARAYGKNSTVHCIIQHEGLHGRKLVAPFQFEDLVADQETTSDAPEQSSLSSQALQQPTSTVSMMENSSIPETDKEEKEHATQDPGLSTASAQHTGLARRKSGILLLTLVSFLIFILFIIVQLFIMKLRKAHVVWKKESEISEQALESYRSRSNNEETSSQENSSQAPQSKRCMNYITQLYSGAKTKKSAQHWKLGGKHSRVPESIV

SEQ ID NO: 5 - protein, 442 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQRDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDHAVIGGVVAVVVFAMLCLLIILGRYFARHKGTYFTHEAKGADDAADADTAIINAEGGQNNSEEKKEYFI

SEQ ID NO: 6 - protein, 398 aa, Homo sapiens
QNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQRDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDHAVIGGVVAVVVFAMLCLLIILGRYFARHKGTYFTHEAKGADDAADADTAIINAEGGQNNSEEKKEYFI

SEQ ID NO: 7 - protein, 456 aa, Mus musculus
MASAVLPSGSQCAAAAAVAAAAAPPGLRLRLLLLLLSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNKELKGKSEVEEWSDMYTVTSQLMLKVHKEDDGVPVICQVEHPAVTGNLQTQRYLEVQYKPQVHIQMTYPLQGLTREGDAFELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDTTATTEPAVHDSRAGEEGTIGAVDHAVIGGVVAVVVFAMLCLLIILGRYFARHKGTYFTHEAKGADDAADADTAIINAEGGQNNSEEKKEYFI

SEQ ID NO: 8 - protein, 409 aa, Mus musculus
QNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNKELKGKSEVEEWSDMYTVTSQLMLKVHKEDDGVPVICQVEHPAVTGNLQTQRYLEVQYKPQVHIQMTYPLQGLTREGDAFELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDTTATTEPAVHDSRAGEEGTIGAVDHAVIGGVVAVVVFAMLCLLIILGRYFARHKGTYFTHEAKGADDAADADTAIINAEGGQNNSEEKKEYFI

SEQ ID NO: 9 - DNA, 21 bp, Artificial sequence
  sequence is synthesized
agggaagacggtgaaagtgaa

SEQ ID NO: 10 - DNA, 21 bp, Artificial sequence
  sequence is synthesized
ccggatggcttcacacagcta

SEQ ID NO: 11 - DNA, 21 bp, Artificial sequence
  sequence is synthesized
cggaacgatattcctgagata

SEQ ID NO: 12 - DNA, 21 bp, Artificial sequence
  sequence is synthesized
ctggagggacttacccaccta

SEQ ID NO: 13 - DNA, 20 bp, Artificial sequence
  sequence is synthesized
gacacaggcaaggtcacaga

SEQ ID NO: 14 - DNA, 22 bp, Artificial sequence
  sequence is synthesized
agagtaactgcccttggacgtg

SEQ ID NO: 15 - DNA, 20 bp, Artificial sequence
  sequence is synthesized
gcagcgcatcgccttctatc

SEQ ID NO: 16 - DNA, 22 bp, Artificial sequence
  sequence is synthesized
gtctgccaagttccttgtacac

SEQ ID NO: 17 - DNA, 22 bp, Artificial sequence
  sequence is synthesized
actgggctctcacttcttaatc

SEQ ID NO: 18 - DNA, 22 bp, Artificial sequence
  sequence is synthesized
ttggttttgggagcttaattct

SEQ ID NO: 19 - DNA, 22 bp, Artificial sequence
  sequence is synthesized
gtatagagctcagggaattgaa

SEQ ID NO: 20 - DNA, 20 bp, Artificial sequence
  sequence is synthesized
ggcagaatggagagaaatcg

SEQ ID NO: 21 - DNA, 20 bp, Artificial sequence
  sequence is synthesized
ccagtgtgagcagcaggata

SEQ ID NO: 22 - DNA, 26 bp, Artificial sequence
  sequence is synthesized
tctacctcagactctttgaagtcttg

SEQ ID NO: 23 - DNA, 22 bp, Artificial sequence
  sequence is synthesized
ggtgtgattcaatgacgcttat

SEQ ID NO: 24 - DNA, 26 bp, Artificial sequence
  sequence is synthesized
aagacaatcaggccatcagcaacaac

SEQ ID NO: 25 - DNA, 19 bp, Artificial sequence
  sequence is synthesized
gacaggttccagccctaca

SEQ ID NO: 26 - DNA, 20 bp, Artificial sequence
  sequence is synthesized
gagctgattgctgagtttgg

SEQ ID NO: 27 - DNA, 22 bp, Artificial sequence
  sequence is synthesized
caggaaaggcaccacctcctgc

SEQ ID NO: 28 - DNA, 21 bp, Artificial sequence
  sequence is synthesized
aaagggctctgacaacacatt

SEQ ID NO: 29 - DNA, 21 bp, Artificial sequence
  sequence is synthesized
tcagaaagtccaccacagttg

SEQ ID NO: 30 - DNA, 25 bp, Artificial sequence
  sequence is synthesized
tgactcatcatcgaattggcactca

SEQ ID NO: 31 - protein, 217 aa, Artificial sequence
  17B2 chimeric hamster-mouse anti-CRTAM antibody light chain
AEVVFTQPQSVSGSLGQEISISCTRSSGNIGNNYVSWYQQQSSNKPRLLIYKDDQRPSGVPDRFSGSTDSSSNSGILTISRLQPEDEGDYYCLSAFDNYFVFGSGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSPAECS

SEQ ID NO: 32 - protein, 446 aa, Artificial sequence
  17B2 chimeric hamster-mouse anti-CRTAM antibody heavy chain
EVQLVESGGGVVQPAKSLKLSCVASGFTFSGAWMSWVRQAPGKGLEWVAQIRDKKNNYATYYGESVSGRLTISRDDSKSTVYLQMNNLRVEDTAVYYCVRLGLDYWGQGTMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - DNA, 800 bp, Artificial sequence
  17B2 chimeric hamster-mouse anti-CRTAM antibody light chain
acctcggttctatcgattgaattccaccatgggatggtcatgtatcatcctttttctagtagcaactgcaaccggtgtacattcagcggaggttgtctttacgcagccccagtctgtgtccggatctttgggccaggagatcagcatttcctgcacacgcagcagtggaaacattggaaacaattatgtatcctggtatcagcaacaatcatcgaacaagccccgtctgctcatctataaagatgatcaaagaccatctggcgttccagacaggttttctggctccacggacagttcctccaactcaggcattttaactatctccaggttgcagcctgaggatgaaggtgactattattgtctgtctgcttttgacaactattttgtttttggcagcgggaccaagctgactgtccttggccaacctaagtcttcaccatctgtcaccctcttcccgccatcttctgaggagttggaaactaacaaagccactcttgtgtgcacgatcactgacttctatcccggagtggtcacagtagactggaaggtggatggcacccccgtaacgcagggcatggagacgactcagccttcgaagcagagcaacaacaaatacatggcgtctagctacctgaccctgaccgcaagagcgtgggagcgccacagtagctactcctgccaagtcacccatgagggccataccgtcgaaaagtccctctccccggcagagtgttcttaagcttggccgccatggcccaacttgtttattgcagcttataatggttacaaataaagcaata

SEQ ID NO: 34 - DNA, 1500 bp, Artificial sequence
  17B2 chimeric hamster-mouse anti-CRTAM antibody heavy chain
acctcggttctatcgattgaattccaccatgggatggtcatgtatcatcctttttctagtagcaactgcaactggagtacattcagaagttcagctggtggagtctggaggaggcgtggtccagcctgcaaagtccctgaaactctcctgtgttgcctctggattcaccttcagtggtgcctggatgagctgggtccgccaggctccagggaaggggctggagtgggttgcacaaattagagacaaaaaaaataattatgcaacatattatggggagtccgtgagcggcagactcaccatctcaagagatgactccaagagcactgtctacctgcaaatgaacaacctaagagtcgaggacaccgccgtttattattgtgtaagactcgggcttgattactggggccaaggaacgatggtcaccgtcagctcagcgaaaacaacagccccatcggtctatccactggctcctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgaaagcttggccgccatggcccaacttgtttattgcagcttataatggttacaaataaagcaatagcatcacaaat

SEQ ID NO: 35 - protein, 112 aa, Artificial sequence
  17B2 antibody light chain variable domain
AEVVFTQPQSVSGSLGQEISISCTRSSGNIGNNYVSWYQQQSSNKPRLLIYKDDQRPSGVPDRFSGSTDSSSNSGILTISRLQPEDEGDYYCLSAFDNYFVFGSGTKLTVLG

SEQ ID NO: 36 - protein, 116 aa, Artificial sequence
  17B2 antibody heavy chain variable domain
EVQLVESGGGVVQPAKSLKLSCVASGFTFSGAWMSWVRQAPGKGLEWVAQIRDKKNNYATYYGESVSGRLTISRDDSKSTVYLQMNNLRVEDTAVYYCVRLGLDYWGQGTMVTVSS

SEQ ID NO: 37 - protein, 13 aa, Artificial sequence
  17B2 antibody light chain CDR1
TRSSGNIGNNYVS

SEQ ID NO: 38 - protein, 7 aa, Artificial sequence
  17B2 antibody light chain CDR2
KDDQRPS

SEQ ID NO: 39 - protein, 9 aa, Artificial sequence
  17B2 antibody light chain CDR3
LSAFDNYFV

SEQ ID NO: 40 - protein, 10 aa, Artificial sequence
  17B2 antibody heavy chain CDR1
GFTFSGAWMS

SEQ ID NO: 41 - protein, 19 aa, Artificial sequence
  17B2 antibody heavy chain CDR2
QIRDKKNNYATYYGESVSG

SEQ ID NO: 42 - protein, 5 aa, Artificial sequence
  17B2 antibody heavy chain CDR3
LGLDY

(19) United States
(12) Patent Application Publication
(10) Pub. No.: US 2010/0247430 A1
(43) Pub. Date: Sep. 30, 2010

(54) Methods and Compositions for Modulating T Cells

(76) Inventors: Andrew Chan, Menlo Park, CA (US); Jung-Hua Yeh, Belmont, CA (US)

Correspondence Address: GENENTECH, INC., 1 DNA WAY, SOUTH SAN FRANCISCO, CA, 94080, US

(21) Appl. No.: 12/675,763

(22) PCT Filed: Aug. 29, 2008
(86) PCT No.: PCT/US08/74908

§ 371 Date: Jun. 11, 2010

Related U.S. Application Data
(60) Provisional application No. 60/969,059, filed on Aug. 30, 2007.
(60) Provisional application No. 61/034,021, filed on Mar. 5, 2008.

(51) Int. Cl. A61K 39/395; A61K 51/10; A61P 37/00; C12Q 1/04; C12N 5/0783; C07K 16/18; C12N 9/96

(52) U.S. Cl. 424/1.49; 424/154.1; 424/133.1; 424/183.1; 435/34; 435/372.3; 530/388.75; 530/387.3; 530/391.7; 435/188

(57) ABSTRACT

The invention provides methods and compositions useful for modulating T cells, and disorders associated with the dysregulation thereof.

### CROSS REFERENCE TO RELATED APPLICATIONS

This application claims the benefit of priority of provisional U.S. Application No. 60/969,059 filed Aug. 30, 2007, and provisional U.S. Application No. 61/034,021 filed Mar. 5, 2008, both of which are incorporated by reference herein in their entirety.

### TECHNICAL FIELD

The invention relates generally to the fields of treatment of immune-related diseases and other pathological conditions. More specifically, the invention concerns methods and compositions for modulating molecular events associated with CRTAM expression in activated T cells.

### BACKGROUND

Coordinate interaction between T cells and APCs is required for efficient TCR activation and development of their effector functions (Dustin and Cooper, 2000; Friedl et al., 2005; Huppa and Davis, 2003; Krummel and Macara, 2006; Vicente-Manzanares and Sanchez-Madrid, 2004). Surface co-receptors, including CD2, CD4, CD8 and CD28, coordinate an initial signaling platform at the T cell:APC contact region to induce changes of membrane dynamics, cell polarity and cell shape required for cytokine production and effector functions. Many TCR-activated genes control subsequent cellular fate and repertoire decisions (Cantrell, 1996). Upregulation of transcription factors, such as Tbx21/T-bet, Gata3, Rorc (γt), and Foxp3, regulate T cell subset differentiation into T_{H}1, T_{H}2, T_{H}17 and Treg cells, respectively (Lee et al., 2006; Tato et al., 2006). Upregulation of cell surface proteins, such as CD25, confers IL2 responsiveness and enables cells to undergo cytokine-mediated cell division (Ma et al., 2006). Conversely, upregulation of CTLA4 modulates T cell responses and contributes to shaping the TCR repertoire (Teft et al., 2006). Hence, the regulated expression of molecules following TCR activation can play important roles in determining T cell responses.

CRTAM, a type I transmembrane protein containing V and C1-like Ig domains (Du Pasquier, 2004), is a cytotoxic or regulatory T cell associated molecule identified as a cell surface marker protein structurally related to the immunoglobulin superfamily and expressed on certain T cells. (Kennedy et al.; U.S. Pat. No. 5,686,257; and WO2006/098887). It has been reported that human CRTAM transcripts are transiently detected in activated CD8⁺ T cells, NK and NKT cells and that interaction between CRTAM and its ligand Necl2 (also known as CADM1) may play a role in antitumor immune responses (Fuchs and Colonna, 2006; Kennedy et al., 2000). Mouse Crtam has been disclosed as an activation-induced gene in double negative (DN) thymocytes (Kennedy et al., 2000). Microarray analysis by Abbas et al. (Genes Immun (2005), 6:319-331; US2007/0184444; US2007/0185017; US2006/0263774) indicated CRTAM as a candidate gene upregulated on human CD4⁺ and CD8⁺ T cells, although the precise T cell sub-population, nature, extent, and physiological significance of CRTAM expression during T cell activation was not clear. The cytoplasmic COOH-terminal sequence of Crtam contains a highly conserved class I PSD-95/Discs-large/ZO-1 (PDZ)-domain protein-interacting motif that facilitates assembly of large protein complexes involved in a variety of signaling pathways including cellular adhesion, polarity and proliferation. In T cells, a network of PDZ-containing proteins, including Scrib and Dlg1, play critical roles in T cell uropod formation, migration and contributes to T cell:APC conjugate formation (Ludford-Menting et al., 2005). Scrib and Dlg1 have been demonstrated to be dynamically regulated following TCR engagement and affect NFAT and cytokine transcriptional activation (Round et al., 2007; Round et al., 2005; Xavier et al., 2004). Scrib serves as a scaffold to assemble signaling complexes that regulate cellular polarity in epithelial and neuronal cells (Humbert et al., 2006). Mutation of scribble (scrib) in Drosophila or knockdown of Scrib in epithelial cells results in loss of polarity, increased cell cycling from G1 to S phase and increased cellular proliferation (Bilder et al., 2000; Nagasaka et al., 2006). However, it is not clear what role, if any, CRTAM plays in activated T cells (such as CD4+ cells) whose dysregulation underlies numerous pathological disorders. Because T cells play critical roles in the normal physiological context, therapeutic modulation of T cell activity would benefit from a fine-tuned targeting of the pathological subset of T cells, while sparing bystander T cells that are required for normal immune function.

It is well-established that T cells are central players in the immune system, and that undesirable perturbation of the delicate balance of T cells in its various stages and forms can lead to serious pathological conditions, including autoimmune diseases where there is unchecked and/or heightened immune responses, or cancer and persistent infections where there is insufficient immune defense. As such, a number of therapeutic agents have been used to treat such conditions. Nonetheless, clinical experience has demonstrated that these agents fall short of the ideal in efficacy and safety characteristics, and are often associated with short and long-term undesirable side effects. For example, it is clear that not all agents are efficacious and/or safe in all patients, thus pointing to a need to develop novel agents that target new molecular entities that are restrictively associated with the underlying pathological causes, for example by adopting a more fine-tuned approach focused on targeting specific subsets of T cells. Such an approach requires identification of targets present on and/or responsible for only the subset of T cells primed and/or physiologically destined to cause the effects underlying a disease of interest.

Molecules suitable for therapeutic targeting include those that are expressed on restricted sub-populations of T cells that mark and/or are responsible for demarcating, from the broad T cell repertoire, the minimum sub-population involved in the etiology or pathology of particular immune-related disorders. Indeed, ideally, such molecules would, in addition to being a restricted T cell marker, also be associated with cellular activities that may be targeted for therapeutic intervention. Unfortunately, to date, information regarding such molecules has been lacking The lack of information has hampered efforts to develop therapeutic strategies that would permit fine-tuned T-cell therapeutic modulation. There is a real need to identify such molecular targets and pathways that could be the focus of novel therapeutic intervention as described above. The invention described herein meets this need and provides other benefits.

All references cited herein, including patent applications and publications, are incorporated by reference in their entirety.

### SUMMARY OF THE INVENTION

The invention is based, at least in part, on the discovery that Crtam is upregulated on a specific subset of T cells (in particular, a specific subset of CD4+ T cells), and that it coordinates a Scrib-centered signaling complex to control a previously unrecognized late phase of T cell polarity and selectively regulates production of certain cytokines, including IFNγ,IL22, and IL17.

Spatial organization of cellular proteins plays an important role in establishment of cellular polarity and is an essential feature for cell division, differentiation, cell migration and organogenesis. Activation of T cells by antigen presenting cells (APCs) results in the formation of an immunological synapse (IS), coordinated assembly of a signaling scaffold at the TCR contact region, reorganization of actin and microtubules and generation of second messengers within the first hours following intercellular contact. As described herein, CRTAM is upregulated on both CD4⁺ and CD8⁺ T cells, and more specifically and unexpectedly, it is upregulated in specific subsets of activated T cells subsequent to a distinct (early) phase of T cell activation, and that it coordinates a distinct set of signaling molecules anchored by the Scrib polarity protein to regulate cell polarity during a late phase of T cell activation to impact cellular division, proliferation and selective cytokine production of the adaptive immune response. CRTAM expression is shown herein to mark specific subsets of the T cell repertoire. CRTAM biological function is shown herein to be linked to effectuating certain activities in T cells during late phase activation, and interference with CRTAM function is demonstrated herein to result in modification of activated T cell activities. Thus, the data disclosed herein reveal an important molecular target/pathway that is specifically associated with an important subset of activated T cells, and that when interfered with, results in modulation of key activities associated with such subset of activated T cells. The invention provides methods, compositions, kits and articles of manufacture useful for regulating the presence and/or activity of such cells, by modulating CRTAM-associated T cell activity, for example by selective targeting and/or ablation of the presence and/or activity of certain CRTAM-expressing T cells.

In one aspect, the invention provides a CRTAM modulator that modulates CRTAM activity in T cells, e.g., activated T cells such as CD4+ T cells. In one embodiment, the CRTAM modulator modulates CRTAM activity associated with binding of cognate ligand (e.g., Necl2) to CRTAM. In one embodiment, the CRTAM modulator modulates CRTAM activity induced by complex formation between CRTAM and Scrib. A CRTAM modulator of the invention may modulate one or more aspects of the CRTAM-Scrib pathway. For example, in one embodiment, a CRTAM modulator of the invention modulates one or more cellular events associated with CRTAM and/or Scrib activity, including, for example, formation and/or maintenance of immunological synapse, T cell polarization, maintenance of T cell activation, etc. For example, in one embodiment, a CRTAM modulator of the invention is capable of antagonizing and/or inhibiting CRTAM activity in activated T cells such as CD4+ T cells. In one embodiment, a CRTAM modulator of the invention is capable of modulating the amount of polarized activated T cells, such as CD4+ T cells, in vitro or in vivo. For example, in one embodiment, a CRTAM modulator of the invention is capable of antagonizing and/or inhibiting CRTAM activity in activated T cells such as CD4+ T cells such that the amount of polarized T cells is decreased. In one embodiment, a CRTAM modulator of the invention is capable of enhancing CRTAM activity in activated T cells such as CD4+ T cells. Thus, for example, in one embodiment, a CRTAM modulator of the invention is capable of enhancing CRTAM activity in activated T cells such as CD4+ T cells such that the amount of polarized T cells is increased.

A CRTAM modulator of the invention can be provided in any form suitable for clinical use. For example, a modulator of the invention can be provided and/or administered in the form of a nucleic acid or polypeptide. Accordingly, in one embodiment, the nucleic acid encodes inhibitory/interfering RNA (such as siRNA) or antisense RNA capable of modulating CRTAM, e.g. by modulating expression of CRTAM. In one embodiment, the nucleic acid encodes or comprises a microRNA sequence capable of modulating CRTAM expression and/or activity. In one embodiment, a modulator of the invention is a molecule capable of interfering with a microRNA such that CRTAM expression and/or activity is modulated in a cell. In one embodiment, a CRTAM modulator of the invention comprises a nucleic acid that encodes a polypeptide capable of antagonizing CRTAM activity. For example, the polypeptide may comprise a peptide comprising at least a portion of CRTAM, wherein the peptide is capable of interacting with CRTAM's interacting partner (e.g., its extracellular or intracellular binding partner). In one embodiment, the modulator polypeptide is a fusion polypeptide comprising at least a portion of CRTAM (e.g., a portion of the extracellular domain of CRTAM capable of binding to a CRTAM ligand such as Necl2) fused to at least a portion of an immunoglobulin sequence (e.g., Ig Fc). In one embodiment, the modulator polypeptide comprises truncated CRTAM comprising a Scrib binding sequence. In one embodiment, the modulator polypeptide is a fusion polypeptide comprising at least a portion of a CRTAM binding partner (e.g., a portion of Necl2 capable of binding to CRTAM) fused to at least a portion of an immunoglobulin sequence (e.g., Ig Fc). In one embodiment, the modulator polypeptide is an extracellular domain of Necl2 fused to Ig Fc. In one embodiment, the modulator polypeptide is an antibody. In one embodiment, the modulator polypeptide is an anti-CRTAM antibody. In one embodiment, the modulator polypeptide is an anti-Necl2 antibody. In one embodiment, a modulator of the invention comprises an aptamer.

Accordingly, in one embodiment, a CRTAM modulator of the invention comprises a nucleic acid that when present in a cell (e.g., an activated CD4+ T cell) inhibits activity (including but not limited to gene expression) of CRTAM in the cell. In one embodiment, the nucleic acid comprises an antisense oligonucleotide. In another embodiment, the nucleic acid comprises an inhibitory/interfering RNA. In one embodiment, the inhibitory/interfering RNA is a small inhibitory/interfering RNA (siRNA). In one embodiment, a CRTAM modulator of the invention comprises an antibody. In one embodiment, an anti-CRTAM antibody may be a monoclonal antibody, including fragments thereof. In one embodiment, an anti-CRTAM antibody may be a polyclonal antibody. In one embodiment, an antibody is blocking or non-blocking, which in each case may also be a depleting antibody. In one embodiment, the antibody is a depleting antibody. In one embodiment, an antibody fragment may, for example, be Fab, Fab′, F(ab′)₂, scFv, (scFv)₂, dAb, complementarity determining region (CDR) fragments, linear antibodies, single-chain antibody molecules, minibodies, diabodies, and multispecific antibodies (e.g., as formed from antibody fragments). In one embodiment, an antibody of the invention has altered effector function, for example, by Fc sequence mutation, alterations of Fc glycosylation (e.g., afucosylation). Methods for altering antibody effector function are varied and well known in the art.

In one embodiment, a CRTAM antibody is a depleting antibody. In one embodiment, the depleting antibody is an antibody conjugate that comprises a toxin. In one embodiment, the toxin is a radioactive isotope, an enzyme, a small molecule toxin, and/or a cytotoxic agent. In one embodiment, a CRTAM modulator of the invention comprises a molecule that interferes with CRTAM interaction with its intracellular binding partner (e.g., Scrib). For example, such molecule may comprise a nucleic acid, a peptide, a small molecule, or an antibody that is administered such that it enters a target T cell to inhibit CRTAM activity in the cell, i.e., the molecule is internalized. In one embodiment, a peptide or antibody is encoded by a nucleic acid that is introduced (e.g., transfected) into a target T cell.

In one aspect, the invention provides a binding agent capable of binding CRTAM. In one embodiment, such binding agent comprises any molecule that is capable of detecting CRTAM present in a sample, or associated with a cell or tissue. In one embodiment, such binding agent is useful for targeting a molecule of interest (e.g., a therapeutic agent, a toxin, etc.) to CRTAM+ subpopulations of T cells. In one such embodiment, a CRTAM+ subpopulation of T cells is CD4+. In one embodiment, a binding agent comprises one or more of the CRTAM modulators described herein.

In one aspect, the invention provides a method of identifying a CRTAM modulator (such as a molecule useful for modulating CRTAM activity in activated T cells), the method comprising contacting a candidate substance with CRTAM (or functional variant thereof as defined hereinbelow), and measuring amount of one or more CRTAM-associated activity as described herein in the presence and absence of the candidate substance, wherein differential amounts of such CRTAM-associated activity in the presence and absence of the candidate substance indicates that the candidate substance is a CRTAM modulator. CRTAM-associated activity can be determined by any suitable qualitative or quantitative measurement known in the art. CRTAM-associated activities include, for example, regulation of cytokine levels, such as IFNγ, interleukin IL22, and/or IL17 in, for example, CD4+ Th1, Th17 and/or CD8+ cells. For example, amount of activity may be indicated by amount of activity of a downstream molecule whose activity is regulated by CRTAM. In another example, amount of activity is indicated by detection and/or measurement of cellular phenotypes (e.g., T cell polarity). Methods of the invention can be used either to identify CRTAM antagonists or agonists. For example, in one embodiment, CRTAM-associated activity is lower in the presence of the candidate substance. In another embodiment, CRTAM-associated activity is higher in the presence of the candidate substance. A modulator identified by a method of the invention can have any of the characteristics required for use in methods of the invention. For example, a candidate can be selected that is capable of modulating T cell activation, and/or maintenance of such activation. In another example, a CRTAM antagonist modulator can be selected that is capable of modulating expression of specific cytokines, including for example inhibiting expression of IFNγ, IL22, and/or IL17. In one aspect, a CRTAM modulator of the invention is obtained by the methods of identifying of the invention as described herein.

As described herein, an antibody of the invention can be in any suitable form for use in a method of the invention. For example, an antibody of the invention can be human, humanized or chimeric. In one embodiment, an antibody of the invention is the humanized or chimeric form of the antibody designated 17B2.7.12.9.2.2 (ATCC deposit no. PTA-8463); 32D6.4.1.1.1 (ATCC deposit no. PTA-8461); 34G4.6.2.1.1.3 (ATCC deposit no. PTA-8460); 6E2.27.8.2.1 (ATCC deposit no. PTA-8462); or 20F4.1.1.1.2.1 (ATCC deposit no. PTA-8464). In one embodiment, an antibody of the invention is a humanized, chimeric or human antibody that binds to the same epitope on CRTAM as the antibody designated 17B2.7.12.9.2.2 (ATCC deposit no. PTA-8463); 32D6.4.1.1.1 (ATCC deposit no. PTA-8461); 34G4.6.2.1.1.3 (ATCC deposit no. PTA-8460); 6E2.27.8.2.1 (ATCC deposit no. PTA-8462); or 20F4.1.1.1.2.1 (ATCC deposit no. PTA-8464). In one embodiment, an antibody of the invention is a humanized, chimeric or human antibody that competes with the antibody designated 17B2.7.12.9.2.2 (ATCC deposit no. PTA-8463); 32D6.4.1.1.1 (ATCC deposit no. PTA-8461); 34G4.6.2.1.1.3 (ATCC deposit no. PTA-8460); 6E2.27.8.2.1 (ATCC deposit no. PTA-8462); or 20F4.1.1.1.2.1 (ATCC deposit no. PTA-8464) for binding to CRTAM (e.g., where CRTAM is located in a cell free environment, or is on a cell (in vitro or in vivo)). In one embodiment, an antibody of the invention comprises one, two, three, four, five or all of the hypervariable regions, hypervariable loops and/or complementarity determining region (CDR) sequences of the antibody designated 17B2.7.12.9.2.2 (ATCC deposit no. PTA-8463); 32D6.4.1.1.1 (ATCC deposit no. PTA-8461); 34G4.6.2.1.1.3 (ATCC deposit no. PTA-8460); 6E2.27.8.2.1 (ATCC deposit no. PTA-8462); or 20F4.1.1.1.2.1 (ATCC deposit no. PTA-8464). In one embodiment, an antibody of the invention comprises one or both variable domains, or functional portion thereof, of the antibody designated 17B2.7.12.9.2.2 (ATCC deposit no. PTA-8463); 32D6.4.1.1.1 (ATCC deposit no. PTA-8461); 34G4.6.2.1.1.3 (ATCC deposit no. PTA-8460); 6E2.27.8.2.1 (ATCC deposit no. PTA-8462); or 20F4.1.1.1.2.1 (ATCC deposit no. PTA-8464). In one embodiment, an antibody of the invention comprises the light chain of the antibody designated 17B2 (SEQ ID NO: 31). In one embodiment, an antibody of the invention comprises the variable region of the light chain of the antibody designated 17B2 (SEQ ID NO: 35). In one embodiment, an antibody of the invention comprises at least one of CDR1 (SEQ ID NO: 37), CDR2 (SEQ ID NO: 38), or CDR3 (SEQ ID NO: 39) of the variable region of the light chain of the antibody designated 17B2. In one embodiment, an antibody of the invention comprises CDR1 (SEQ ID NO: 37), CDR2 (SEQ ID NO: 38), and CDR3 (SEQ ID NO: 39) of the variable region of the light chain of the antibody designated 17B2. In one embodiment, an antibody of the invention comprises the heavy chain of the antibody designated 17B2 (SEQ ID NO: 32). In one embodiment, an antibody of the invention comprises the variable region of the heavy chain of the antibody designated 17B2 (SEQ ID NO: 36). In one embodiment, an antibody of the invention comprises at least one of CDR1 (SEQ ID NO: 40), CDR2 (SEQ ID NO: 41), or CDR3 (SEQ ID NO: 42) of the variable region of the heavy chain of the antibody designated 17B2. In one embodiment, an antibody of the invention comprises CDR1 (SEQ ID NO: 40), CDR2 (SEQ ID NO: 41), and CDR3 (SEQ ID NO: 42) of the variable region of the heavy chain of the antibody designated 17B2. In one embodiment, an antibody of the invention is the humanized, chimeric or human form of an affinity-matured derivative of the antibody designated 17B2.7.12.9.2.2 (ATCC deposit no. PTA-8463); 32D6.4.1.1.1 (ATCC deposit no. PTA-8461); 34G4.6.2.1.1.3 (ATCC deposit no. PTA-8460); 6E2.27.8.2.1 (ATCC deposit no. PTA-8462); or 20F4.1.1.1.2.1 (ATCC deposit no. PTA-8464).

In one aspect, the invention provides methods for the prevention or treatment of disease, for example an autoimmune disease, comprising administering to a subject an effective amount of a CRTAM modulator of the invention. In one embodiment, the subject is a mammalian subject, for example, a human subject. In one embodiment, the CRTAM modulator is a CRTAM antagonist.

In one embodiment, a disease treated and/or prevented by a method of the invention is characterized by the presence of activated CD4⁻CRTAM⁺ T cells. In one embodiment, the T cells are also characterized by elevated levels of cytokine expression, when compared to expression in CD4⁻CRTAM⁻ T cells. In yet another embodiment, the T cells are characterized by elevated cytokine secretion levels when compared to cytokine secretion levels of CD4⁺CRTAM⁻ T cells. In one embodiment, the cytokine is IFNγ, IL22, and/or IL17. In one embodiment, the T cells are autoreactive.

In one embodiment, a method of treatment or prevention of a disease is characterized by a decrease in cytokine expression in the subject following administration of a CRTAM modulator of the invention as compared to a subject not administered said CRTAM modulator. In another embodiment, the treatment or prevention is also characterized by a decrease in cytokine secretion levels in the subject following administration of a CRTAM modulator of the invention as compared to a subject not administered said CRTAM modulator. In one embodiment, the cytokine is IFNγ, IL22, and/or IL17. In one embodiment, the T cells are autoreactive.

In another aspect, the invention provides a method of inhibiting T cell proliferation or an effector function of T cells. In one embodiment, T cells are activated T cells, such as CD4+ T cells. In one embodiment, the invention provides a method of inhibiting T cell proliferation or an effector function of T cells comprising contacting a CRTAM modulator with a T cell, wherein the modulator inhibits CRTAM activity in the T cell, for example by inhibiting binding of a ligand to CRTAM in said T cell. In one embodiment, the modulator comprises an antibody as described herein, including a blocking or non-blocking antibody, which in each case may also be a depleting antibody. In one embodiment, the antibody is a depleting antibody. In one embodiment, the antibody is an antibody fragment. In one embodiment, the antibody is an antibody conjugate that comprises a toxin. In one embodiment, the toxin comprises a radioactive isotope, an enzyme, a small molecule toxin, and/or an agent having cytotoxic activity.

The invention provides methods of treating or preventing a disease using a CRTAM modulator of the invention that is capable of modulation of a CRTAM biological activity as described herein. In one embodiment, the modulation relates to one or more cellular events including, without limitation, one or more of the following: cell proliferation, cycling and/or division; cell adhesion; development of T cell effector function; cytokine production; intracellular recruitment to the membrane of certain molecules including, without limitation, one or more of the following: Scrib, PKCζ, and Cdc42; intracellular coordination of the assembly of a Cdc42-containing complex at the leading edge of T cells; later stage cytoskeletal reorganization; and T cell polarity. In one embodiment, the modulation by a CRTAM modulator may affect certain CRTAM⁺ cell types including, without limitation, one or more of the following: CD4⁺ T cells (e.g., Th1 and Th17 cells), CD8⁺ T cells, NK cells, and NKT cells. In another embodiment, the modulation by a CRTAM modulator involves an effect on cytokine production including, without limitation, one or more of the following cytokines IFNγ, IL22, and/or IL17.

In one embodiment, a method of treating or preventing a disease of the invention comprises depletion of a T cell sub-population associated with administering to a subject a CRTAM modulator as provided herein. In one embodiment, the depleted T cell population is a CRTAM⁺ CD4+ T cell sub-population, for example, Th1 and/or Th17 T cell sub-population.

In one aspect, the invention provides a method of detecting or diagnosing a disease in a subject comprising (a) contacting a CRTAM modulator (e.g., a CRTAM binding agent) with a first sample of cells (e.g., T-cells) obtained from the subject, (b) comparing amount of CRTAM⁺ cells in the sample as compared to a control sample, wherein a higher amount in the first sample is indicative of the presence of the disease in the subject. In one embodiment, the method of detecting a disease further comprises the steps of (c) obtaining a second sample containing T cells from the subject; (d) contacting a CRTAM modulator (e.g., a CRTAM binding agent) with the second sample; and (e) comparing amount of CRTAM⁺ cells in the first and second sample, where a higher amount in the second sample is indicative of a flare-up in the disease (e.g., autoimmune disease) in the subject.

In one embodiment, a sample(s) from a subject may contain T cells, including without limitation, one or more of the following CRTAM+ T cells, such as CD4⁺ cells (e.g., Th1 or Th17), and/or CD8⁺ cells. In one embodiment, an immunological disorder that is detected/diagnosed by a method of the invention is an active autoimmune disease. In one embodiment, such active autoimmune disease is characterized by the presence of activated T cells, where the T cells are CRTAM⁺. In one embodiment, the T cells are CD4⁺ and/or CD8+. In one embodiment, the CD4⁺ cells are Th1 or Th17. In one embodiment, the activated T cells may also be characterized by elevated levels of cytokine expression as compared to such cytokines' expression in CRTAM⁻ T cells, and in one embodiment, such cells are further characterized by elevated cytokine secretion levels as compared to such cytokines' secretion levels in CRTAM⁻ T cells. In one embodiment, the T cells are CD8+ or CD4+ (e.g., Th1 or Th17). In one embodiment, such activated T cells are autoreactive. These autoreactive T cells may be activated CD4+ T cells or CD8+ T cells. In one embodiment, the amount of polarized T cells (e.g., as a ratio to other cells) is increased compared to a non-disease reference. In one embodiment, these cells are associated with etiology and/or pathology of autoimmune diseases.

In one aspect, the invention provides methods for the preparation, isolation, and/or purification of substantially pure populations of activated T cells. In one embodiment, the population comprises activated CD4+ T cells characterized by expression of CRTAM. In one embodiment, such T cells exhibit an elevated level of cytokine expression and/or secretion relative to CD4+ activated T cells not expressing CRTAM. In one embodiment, the cytokine is IFNγ, IL22, and/or IL17. In one embodiment, the methods of preparation, isolation, and/or purification comprise contacting a sample known or suspected to comprise a mixed population of T cells with a CRTAM binding agent (e.g., an anti-CRTAM antibody), and separating any cells from the mixed population that do not substantially bind the CRTAM binding agent (e.g., a CRTAM antibody), whereby a population of activated CD4+ T cells bound by the CRTAM binding agent (e.g., CRTAM antibody) is isolated. The method may further comprise separating the bound CRTAM binding agent from the population of activated CD4+ cells that is bound to the CRTAM binding agent.

In one aspect, the invention provides a composition comprising a substantially pure population of activated T cells. In one embodiment, the population comprises activated CD4+ T cells characterized by expression of CRTAM. In one embodiment, such T cells exhibit an elevated level of cytokine expression and/or secretion relative to CD4+ activated T cells not expressing CRTAM. In one embodiment, the cytokine is IFNγ, IL22, and/or IL17.

In one other aspect, the invention provides use of a CRTAM modulator in the preparation of a medicament to treat a disease, e.g., an autoimmune disease.

In one aspect, the invention provides a CRTAM modulator for use in the treatment of a disease, e.g., an autoimmune disease. In one aspect, the invention provides methods for treating a variety of disorders associated with dysregulation of T cell activation and/or function. For example, in one aspect, the invention provides a method of treating a disorder associated with abnormal T cell activation and/or function, said method comprising administering to a subject an effective amount of a CRTAM modulator of the invention, thereby treating the disorder. In one embodiment, the modulator decreases T cell activation. In one embodiment, the modulator decreases T cell activity, which in one embodiment is autoreactive activity. In one embodiment, the modulator inhibits inflammation associated with dysregulation of T cell activation.

In one aspect, the invention provides a method of treating a disorder by enhancing T cell activity (in particular, CD4+ T cell activity), said method comprising administering to a subject with the disorder an effective amount of a CRTAM modulator that enhances CRTAM actvitiy, whereby the disorder is treated. In one embodiment, the CRTAM modulator comprises an agonist molecule, for example an agonist antibody or a fusion polypeptide comprising a CRTAM-binding ligand (e.g., at least a portion of the CRTAM-binding domain of Necl2 fused to an immunoglobulin sequence such as an immunoglobulin Fc). Such methods of the invention are useful for treating disorders that would benefit from enhancement of activated CD4+ T cell activity, e.g. cancer, immune deficiencies, and infections.

In methods of the invention wherein a CRTAM modulator of the invention is administered in conjunction with another therapeutic agent, the timing of administration of CRTAM modulator relative to the timing of administration of said another therapeutic agent would be any that is deemed empirically and/or clinically to be therapeutically beneficial. For example, in one embodiment, the CRTAM modulator is administered prior to administration of said another therapeutic agent. In one embodiment, the CRTAM modulator is administered subsequent to administration of said another therapeutic agent. In another embodiment, the CRTAM modulator is administered concurrently with administration of said another therapeutic agent. In one embodiment, said another therapeutic agent has anti-inflammatory activity. In one embodiment, said another therapeutic agent has immunosuppresive activity. In one embodiment, said another therapeutic agent has cytotoxic activity. In one embodiment, said another therapeutic agent has anti-infective activity. In one embodiment, the two agents are administered as separate compositions. In one embodiment, the two agents are administered as a single composition.

Where clinically appropriate or desired, methods of the invention can further comprise additional treatment steps. For example, in one embodiment, a method further comprises a step wherein a targeted cell and/or tissue is exposed to other standard of care treatment regimens (e.g., steroids, or other polypeptide or small molecule anti-inflammatory agents).

As noted above, CRTAM modulators and methods of the invention are useful in treating a variety of disorders suspected or known to be associated with improper regulation of T cell activity. For example, these disorders include, but are not limited to, those that are in the immunological (such as autoimmune), cancer or infection-related categories of disorders. In one embodiment, the disorders are associated with tissue inflammation (acute or chronic), for example autoimmune disorders. It should also be noted that some diseases may have characteristics that overlap between two or more categories of disorders.

In one embodiment, an autoimmune disease may be, for example, rheumatoid arthritis (RA); multiple sclerosis (MS); systemic lupus erythematosus (SLE); lupus nephritis; cutaneous lupus erythematosus (CLE); autoimmune hepatitis; juvenile rheumatoid arthritis; infectious hepatitis; primary biliary cirrhosis; psoriasis; dermatitis; atopic dermatitis; systemic scleroderma; systemic sclerosis; Crohn's disease, ulcerative colitis; respiratory distress syndrome; adult respiratory distress syndrome; ARDS; meningitis; encephalitis; uveitis; glomerulonephritis; pemphigus; macrophage activation syndrome; eczema; asthma; atherosclerosis; leukocyte adhesion deficiency; diabetes mellitus; Type I diabetes mellitus; insulin dependent diabetes mellitis; allergic rhinitis; autoimmune reaction associated with organ transplantation; Reynaud's syndrome; autoimmune thyroiditis; Hashimoto's thyroiditis; allergic encephalomyelitis; Sjogren's syndrome; juvenile onset diabetes; immune responses associated with acute and delayed hypersensitivity mediated by cytokines and T-lymphocytes; tuberculosis, sarcoidosis, polymyositis, granulomatosis; vasculitis; pernicious anemia (Addison's disease); diseases involving leukocyte diapedesis; central nervous system (CNS) inflammatory disorder; multiple organ injury syndrome; hemolytic anemia; cryoglobinemia; Coombs positive anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) and autoimmune thrombocytopenia.

In one embodiment, an autoimmune disease may be, more specifically, rheumatoid arthritis (RA), multiple sclerosis (MS), systemic lupus erythematosus (SLE), cutaneous lupus erythematosus (CLE), psoriasis, Crohn's disease, ulcerative colitis, uveitis, atopic dermatitis, asthma, autoimmune reaction associated with organ transplantation, autoimmune hepatitis, juvenile rheumatoid arthritis, infectious hepatitis, glomerulonephritis, primary biliary cirrhosis, vasculitis, pemphigus, macrophage activation syndrome, allergic rhinitis, diabetes mellitus 1, and diabetes mellitus 2.

In one aspect, the invention provides compositions comprising one or more CRTAM modulators of the invention and a carrier. In one embodiment, the carrier is pharmaceutically acceptable.

In one aspect, the invention provides nucleic acids encoding a CRTAM modulator of the invention. In one embodiment, a nucleic acid of the invention encodes a CRTAM modulator which is or comprises a polypeptide (e.g., an oligopeptide). In one embodiment, a nucleic acid of the invention encodes a CRTAM modulator which is or comprises an antibody or fragment thereof. In one embodiment, a nucleic acid of the invention encodes a nucleic acid sequence that inhibits CRTAM expression/activity (e.g., CRTAM gene transcription or translation of CRTAM protein), e.g., siRNA, antisense oligonucleotides, etc.

In one aspect, the invention provides vectors comprising a nucleic acid of the invention.

In one aspect, the invention provides host cells comprising a nucleic acid or a vector of the invention. A vector can be of any type, for example a recombinant vector such as an expression vector. Any of a variety of host cells can be used. In one embodiment, a host cell is a prokaryotic cell, for example, E. coli. In one embodiment, a host cell is a eukaryotic cell, for example a mammalian cell such as Chinese Hamster Ovary (CHO) cell. In one embodiment, a host cell is genetically engineered to produce antibodies having altered effector function, for example where antibodies produced by the cell comprise glycosylation profiles associated with a desired effector function alteration (e.g., as observed in afucosylated antibodies). In one embodiment, a nucleic acid or vector of the invention is expressed in an activated T cell.

In one aspect, the invention provides methods for making a CRTAM modulator of the invention. For example, the invention provides a method of making a CRTAM modulator which is or comprises an antibody (or fragment thereof), said method comprising expressing in a suitable host cell a recombinant vector of the invention encoding said antibody (or fragment thereof), and recovering said antibody (or fragment thereof). In another example, the invention provides a method of making a CRTAM modulator which is or comprises a polypeptide (such as an oligopeptide), said method comprising expressing in a suitable host cell a recombinant vector of the invention encoding said polypeptide (such as an oligopeptide), and recovering said polypeptide (such as an oligopeptide).

In one aspect, the invention provides an article of manufacture comprising a container; and a composition contained within the container, wherein the composition comprises one or more CRTAM modulators of the invention. In one embodiment, the composition comprises a nucleic acid of the invention. In one embodiment, a composition comprising a CRTAM modulator further comprises a carrier, which in some embodiments is pharmaceutically acceptable. In one embodiment, an article of manufacture of the invention further comprises instructions for administering the composition (e.g., the CRTAM modulator) to treat a disorder indicated herein.

In one aspect, the invention provides a kit comprising a first container comprising a composition comprising one or more CRTAM modulators of the invention; and a second container comprising a buffer. In one embodiment, the buffer is pharmaceutically acceptable. In one embodiment, a composition comprising a CRTAM modulator further comprises a carrier, which in some embodiments is pharmaceutically acceptable. In one embodiment, a kit further comprises instructions for administering the composition (e.g., the CRTAM modulator) to treat a disorder indicated herein.

### BRIEF DESCRIPTION OF THE DRAWINGS

FIG. 1. Crtam is expressed upon TCR activation on a sub-population of CD4⁺ T cells that produce more IFNγ, IL22 and IL17.

(A) Flow cytometric analysis of Crtam expression of splenic CD4⁺CD62L⁺ T cells following activation with anti-CD3 (10 μg/ml) and anti-CD28 (2 μg/ml) mAbs.

(B) Splenic naïve CD4⁺ (CD4⁺CD62L⁻) T cells from C57BL/6 mice were activated with anti-CD3 (2 μg/ml) and anti-CD28 (2 μg/ml) mAbs. 14 h later, Crtam⁺ and Crtam⁻ T cells were purified by FACS sorting. Sorted Crtam⁺ and Crtam⁻ T cells were rested for 3 days and re-stimulated at a concentration of 5×10⁵ cells/ml with anti-CD3/28 mAbs. Expression of Crtam on re-stimulated T cells was then examined by flow cytometry.

(C) Splenic naïve CD4⁺ T cells were activated with anti-CD3/28 mAbs for 14 h. mRNA was extracted from sorted Crtam⁺ and Crtam⁻ T cells and subjected to quantitative TaqMan analysis (upper panel). Sorted cells were rested and re-stimulated as described in (B) and supernatants harvested at 48 h and analyzed by ELISA (lower panel).

(D, F, G) Splenic naïve CD4⁺ (CD4⁺CD620⁺) T cells from C57BL/6 mice were stimulated with anti-CD3/28 mAbs under T_{H} differentiating conditions as described. 14 h following TCR activation, Crtam⁺ and Crtam⁻ T cells were purified by FACS sorting. Sorted Crtam⁺ and Crtam⁻ T cells were rested for 3 days and re-stimulated at a concentration of 5×10⁵ cells/ml with anti-CD3 (10 μg/ml) and anti-CD28 (2 μg/ml) mAbs. Supernatants from re-stimulated T cells were harvested at 48 h and analyzed by ELISA.

(E) CD4⁺ T cells were activated under T_{H}1 condition. 14 hrs later, Crtam⁻ and Crtam⁺ CD4⁺ T cells were purified by FACS sorting and thereafter cultured in T_{H}1 conditional media for 4 days. Differentiated T cells were then re-stimulated with PMA and ionomycin in the presence of GolgiPlug (BD Pharmingen) for 4 h. Cells were then fixed and permeabilized by using BD Cytofix/Cytoperm™ Plus Kit for immunofluorescent staining of intracellular IFNγ. FACS analysis shown is representative of five (in A and B) and three (in E) independent experiments. ELISA data shown are representative of five independent experiments in which cells were purified from thirty mice. Error bars indicate the standard deviation (SD). Statistical analysis was performed with a control using Dunnett's Method.

FIG. 2. Crtam^{-/-} CD4⁺ and CD8⁺ T cells have defects in cytokine production.

(A and B) Crtam expression of 12 h-activated Crtam^{+/+} and Crtam^{-/-} T cells was analyzed by flow cytometry (A) and Western blot analysis (B).

(C and D) Naïve CD4⁺ T cells were purified from Crtam^{+/+} and Crtam^{-/-} mice and were activated under T_{H}0 or T_{H}1 conditions. After 6 days, T cells were washed, re-activated and cultured in normal media for three additional days. On day 10, differentiated T cells were stimulated with PMA/Ionomycin for 4 h for TaqMan analysis (C) and PMA/Ionomycin plus GolgiPlug for intracellular staining of IFNγ (D). Data shown are representative of two independent experiments. (E) Naïve Crtam^{+/+} and Crtam^{-/-} CD4⁺ T cells were activated and cultured in T_{H} differentiation media. Six days later, cells were washed, counted and re-stimulated in normal media at a concentration of 5×10⁵ cells/ml with anti-CD3 (10 μg/ml) and anti-CD28 (2 μg/ml) mAbs. Supernatants were collected 48 h after stimulation and analyzed by ELISA.

(F) Naïve (CD8⁺CD62L⁻) and effector/memory (CD8⁺CD62L⁻) CD8⁺ T cells were purified and stimulated at a concentration of 1×10⁶ cells/ml with anti-CD3 (10 μg/ml) and anti-CD28 (2 μg/ml) mAbs for naïve T cells and 5×10⁵ cells/ml with anti-CD3 (5 μg/ml) and anti-CD28 (2 μg/ml) mAbs for effector/memory T cells. Cytokine production was analyzed by ELISA 40 h following activation. Data shown in (E) and (F) are representative of three independent experiments, in which cells were purified and stimulated independently from Crtam^{+/-} (n=3) and Crtam^{-/-} (n=3) mice.

(G) Crtam^{-/-} mice were backcrossed to C57/BL-6 genetic background by Speedy congenic strategy. 10-13 weeks-old mice from N4 generation (Crtam^{+/+}, n=5; Crtam^{-/-}, n=6) were orally inoculated with 2×10⁹ CFU of C. rodentium in 200 μl PBS. Numbers of viable bacteria in the distal colon and spleen were determined on MacConkey agar 7 and 14 days post-infection. In this figure, error bars indicate the standard deviation (SD). Statistical analysis was performed with a control using Dunnett's Method.

FIG. 3. Hyperproliferation of Crtam^{-/-} naïve T cells in response to APC-peptides stimulation.

(A) Naïve OT-II TCR⁻ CD4⁺ T cells from Crtam^{+/+} (open bars) and Crtam^{-/-} (solid bars) mice were activated with irradiated OVA peptide-loaded APCs and cellular proliferation assessed by [³H]-thymidine incorporation.

(B) Cell division was monitored by flow cytometric analysis of carboxyfluorescein diacetate succinimidyl ester (CFSE)-labeled OT-II TCR⁺ CD4⁺ T cells (5 μM OVA peptides). % cells within each division are represented above each histogram.

(C) Naïve OT-I TCR⁺ CD8⁺ T cells from Crtam^{+/+} and Crtam^{-/-} mice were activated with irradiated OVA peptide-loaded APCs and cellular division assessed by [³H]-thymidine incorporation.

(D) Dilution of CFSE-labeled OT-I TCR⁺ CD8⁺ T cells was examined 3 days following stimulation (10 μM OVA peptides). Data shown in A to D are representative of three independent experiments.

(E) Enhanced in vivo expansion of OT-II TCR⁺ Crtam^{-/-} CD4⁺ T cells. CFSE-labeled OT-II TCR⁺ CD4⁺ T cells (8×10⁶ cells/mouse) from Crtam^{-/+} and Crtam^{-/-} mice were transferred into B6129SF2/J mice and challenged with 20 μg OVA protein by footpad injection 12 h following T cell transfer. Draining lymph nodes of recipient mice were analyzed for cell cycling three days following antigen challenge. This data was representative of four independent experiments.

(F) Total CD4⁺ and CD8⁺ T cells, and B220⁺ B-cells from cervical lymph nodes (LN), spleen and blood were quantified from Crtam^{+/+} (open bars) and Crtam^{-/-} (solid bars) mice (age 6 weeks, n=16; age 10 months, n=10). Error bars indicate the standard deviation (SD). Statistical analysis was performed with a control using Dunnett's Method.

FIG. 4. Disruption of a late phase of T cell polarity in activated Crtam^{-/-} CD4⁺ T cells. a-c, (A to C) Naïve Crtam^{-/+} (upper panels) or Crtam^{-/-} (lower panels) CD4⁺ T cells, either unstimulated or stimulated with anti-CD3 (10 μg/ml) and CD28 (2 μg/ml) mAbs for 14 h, were stained for Crtam and Talin (A), CD3ε (B), Crtam and CD44 (C) and analyzed by deconvolution microscopy.

(D) Quantitation of Talin, CD44, and CD3 polarization by cell counting (n>200) using deconvolution microscopy (DeltaVision).

(E) 3D reconstitution of Crtam, Talin, and CD44 staining of 14 h anti-CD3/28 activated T cells using Imaris 5.5 from >35 of 2D slats.

(F) Naïve OT-II TCR⁺ Crtam^{+/+} or Crtam^{-/-} CD4 T cells, either unstimulated or stimulated with OVA peptide-pulsed DC for 14 h, were stained for Talin, CD44 and CD3, and analyzed by deconvolution microscopy.

(G) Quantitation of Talin, CD44, and CD3 polarization in activated OT-II TCR⁺ Crtam^{+/+} and Crtam^{-/-} CD4⁺ T cells (n>200).

(H) Analysis of initial T cell contact formation. CMRA-labeled and OVA peptide-pulsed DC were incubated with OT-II TCR⁺ Crtam^{-/+} and Crtam^{-/-} CD4⁺ T cells for 30 min and stained for F-actin (phalloidin).

(I) Cells were activated as described in (H) and the ability of either Crtam^{+/+} or Crtam^{-/-} OT-II TCR⁺ CD4⁺ T cells to form cell conjugates with OVA peptide-pulsed DCs were analyzed by flow cytometry. Data shown are representative of four independent experiments. Error bars indicate the standard deviation (SD).

FIG. 5. Crtam controls cell polarity, proliferation, and cytokine production through PDZ protein networks.

(A) Naïve CD4⁺ T cells from Crtam^{-/+} and Crtam^{-/-} mice were activated for the indicated times and cell extracts were immunoprecipitated with Abs against Scrib (upper panels) or Dlg1 (lower panels). Immune complexes were then analyzed by immunoblotting against Crtam. Data shown are representative of five independent experiments.

(B to D) Naïve CD4⁺ T cells were activated with anti-CD3 (10 μg/ml) and anti-CD28 (2 g/ml) mAbs for 14 h. Activated cells were then stained for Crtam, Scrib, Cdc42 or PKCζ and analyzed by deconvolution microscopy. Images are representative of >300 cells for each staining.

(E) Naïve OT-II TCR⁺ CD4⁺ T cells were incubated with DCs pulsed with the OVA peptide, fixed at defined time points, adhered onto 8-well chamber slides, and stained with specific antibodies as indicated above each panel. Images are representative of >50 cells for each staining at each time point.

FIG. 6. Interaction of Crtam with Scrib is requisite to control cell polarity, proliferation and cytokine production.

(A) Schematic depiction of structure of Crtam and Crtam mutants.

(B and C) Cellular proliferation and cytokine production of retroviral reconstituted Crtam, Crtam (ΔICD), or Crtam (ΔESIV) in Crtam^{-/-} CD4⁺ T cells. Crtam^{+/+} and Crtam^{-/-} CD4⁻ T cells reconstituted with GFP served as controls. Data are representative of five independent experiments.

(D) Polarity of Crtam, Crtam (ΔICD), or Crtam (ΔESIV) reconstituted Crtam^{-/-} CD4 T cells was examined at 14 h after TCR re-stimulation.

(E and F) FACS sorted Crtam⁺ and Crtam⁻CD4 T cells from C57BL/6 mice were retrovirally transduced with a GFP control, Crtam, or Crtam (ΔESIV). All transfected cells were rested for 3 days and re-stimulated at 2×10⁵ cells/ml with anti-CD3 (10 μg/ml) and anti-CD28 (2 μg/ml) mAbs. Cytokine production was analyzed by ELISA 48 h following TCR restimulation (E). ELISA data are representative of four independent experiments. Error bars indicate the standard deviation (SD) and statistical analysis was performed with a control using Dunnett's Method. Talin polarity in Crtam⁺CD4⁺ T cells (panel 1) or retroviral reconstituted Crtam⁻CD4⁺ T cells (panels 2-4) were analyzed 14 h after re-stimulation with anti-CD3/28 mAbs (F). Images are representative of >100 cells examined for each staining

FIG. 7. Crtam function is transmitted through Scrib.

(A and B) Crtam⁺ CD4 T cells from C57/BL6 mice were purified from 14 h-activated T cells by FACS sorting. Sorted Crtam⁺ CD4 T cells were expanded for 4 days, and were electroporated with control or Scrib siRNA (QIAGEN) by Amaxa Nucleofector. 12 hours following transfection, Scrib expression was analyzed by Western blotting (A). Arrow in (A) indicates endogenous Scrib. T cells were re-stimulated with anti-CD3 and CD28 mAbs for 8 h and stained for Talin (B).

(C) Cytokine production by control or Scrib siRNA transfected cells was analyzed by ELISA 48 h after restimulation. Data shown are representative of two independent experiments.

(D and E) Naïve Crtam^{-/-} CD4⁺ T cells were purified and activated with plate-bound anti-CD3 and anti-CD28 mAbs. Four days following stimulation, T cells were electroporated with pIRES-GFP or pIRES-Crtam(ΔICD):Scrib using Amaxa Nucleofector. Expression of Crtam(ΔICD):Scrib was confirmed by Western blot analysis (D) and flow cytometry (E). Arrowheads indicate the Crtam(ΔICD):Scrib chimera with its differentially glycosylated forms. Arrow in (D) indicates endogenous Scrib.

(F) 12 hours following transfection, T cells were re-stimulated with anti-CD3 and CD28 mAbs for 8 h and cells fixed for staining

(G) Supernatants of cell cultures were collected at 48 hours following TCR stimulation for ELISA analysis. Data shown are representative of three independent experiments.

FIG. 8. Crtam is expressed upon TCR activation on T cells.

Flow cytometric analysis of Crtam expression of splenic CD8⁺CD62L⁻ T cells following activation with anti-CD3 (10 μg/ml) and anti-CD28 (2 μg/ml) mAbs. Data are representative of three independent experiments.

FIG. 9. Generation of Crtam^{-/-} mice.

(A) Gene targeting strategy. Genomic structure surrounding exon 1 of Crtam (top), targeting vector (middle) and the targeted allele (bottom) are depicted. Exon 1 of Crtam was replaced with a neomycin resistance gene by homologous recombination. Arrows indicate primers used for PCR genotyping. Positions of probes used for Southern blotting are shown as gray bars.

(B) Genotyping of targeted alleles (+/+: 328 by and −/−: 191 bp) using PCR amplification (left) and Southern blot analysis (right). The 5′probe hybridized EcoRI fragments of 10.5 kB for +/+ mice and 7.6 kB for −/− mice. The 3′ probe hybridized EcoR1 fragments of 10.5 kB for +/+ mice and 2.8 kB for −/− mice.

(C) Absence of Crtam mRNA expression in Crtam^{-/-} mice. Total RNA of spleen from Crtam^{+/-} and Crtam^{-/-} mice were extracted, reverse transcribed to cDNA and amplified with primers specific to Crtam (exons 4 to 8) or actin.

FIG. 10. Normal T cell development in Crtam^{-/-} mice.

(A) Thymocyte subsets, CD4⁻CD8⁻ (DN), CD4⁻, CD8⁺, and CD4⁺CD8⁺ (DP) were quantified from Crtam^{/+} (open bars) and Crtam^{-/-} (solid bars) mice (age 6 weeks, n=16).

(B) Thymocyte subsets from OT-II TCR⁺ Crtam^{+/+} (open bars) and Crtam^{-/-} (solid bars) mice (age 6 weeks, n=10) were quantified.

(C and D) Total CD4⁺ and CD8⁺ T cells, B220⁺ B-cells, CD4⁺ naive (CD62L^{hi}), CD4⁺ effector)(CD44^{hi}CD45RB^{hi}CD62L^{lo}, CD4⁺ memory)(CD44^{hi}CD45RB^{lo}CD62L^{lo}), CD8⁺ naive (CD62L^{hi}), and CD8⁺ effector)(CD43-1B11^{hi}CD62L^{lo}) T cells from spleen and blood were quantified from Crtam^{+/+} (open bars) and Crtam^{-/-} (solid bars) mice (age 6 weeks, n=16). Representative flow cytometric analysis are shown on top. Error bars indicate the standard deviation (SD). T=total cells; N=naïve cells; E=effector cells; M=memory cells.

FIG. 11. Reduced IFNγ secretion by Crtam^{-/-} CD4⁺ T cells in response to TCR stimulation.

Naive wildtype and Crtam^{-/-} CD4 T cells were purified and stimulated with plate-bound anti-CD3 (1-10 μg/ml) and anti-CD28 (2 μg/ml) mAbs in T_{H}1 differentiation media. Six days later, cells were washed, counted and re-stimulated in normal media at a concentration of 5×10⁵ cells/ml with anti-CD3 (1-10 μg/ml) and anti-CD28 (2 μg/ml) mAbs. Supernatants were collected 48 h after stimulation and analyzed by ELISA. This experiment was representative of two independent experiments. Error bars indicate the standard deviation (SD). Statistical analysis was performed with a control using Dunnett's Method.

FIG. 12. Reduction of IL22 with IL23 treatment.

Naive Crtam^{+/+} and Crtam^{-/-} CD4⁺ T cells were activated and cultured in T_{H}17 differentiation media containing recombinant mouse IL23 (10 ng/ml, eBioscience), anti-IFNγ mAb (5 μg/ml, BD Pharmingen) and anti-IL4 mAb (5 μg/ml, BD Pharmingen). Six days later, cells were washed, counted and re-stimulated in normal media. Supernatants were collected 48 h after stimulation and analyzed by ELISA. Data shown are representative of three independent experiments in which cells were purified and stimulated independently from Crtam^{+/-} (n=3) and Crtam^{-/-} (n=3) mice. Error bars indicate the standard deviation (SD). Statistical analysis was performed with a control using Dunnett's Method.

FIG. 13. Hyperproliferation of Crtam^{-/-} naive CD4⁺ T cells following TCR activation.

(A) Naive CD4⁺ T cells from Crtam^{+/+} (black) and Crtam^{-/-} (grey) mice were activated with plate-bound anti-CD3/CD28 mAbs. Cell proliferation was analyzed by [³H]-thymidine incorporation.

(B) Cell division was monitored with CFSE-labeled CD4⁺ T cells by flow cytometry 3 days after activation.

(C and D) The same experiments were performed with naive CD8⁺ T cells from Crtam^{+/-} (black) and Crtam^{-/-} (grey) mice. The data shown here are representative of four independent experiments.

(E) Naive CD4⁺ T cells from C57BL/6 mice were activated with plate-bound anti-CD3and anti-CD28 mAbs. Crtam^{hi} and Crtam⁻ CD4 T cells were purified by FACS sorting. After four days, cells were restimulated with plate-bound mAbs and cell proliferation was analyzed by [³H]-thymidine incorporation. Data shown here are representative of two independent experiments. Error bars in this figure indicate the standard deviation (SD). Statistical analysis was performed with a control using Dunnett's Method.

(F) Purified CD62L⁺CD4⁺ naive splenic T cells from C57BL/6 (CD45.2⁻) and congenic B6.SJL (CD45.1⁺) mice were stimulated with plate-bound anti-CD3 and anti-CD28 mAbs. Fourteen hours later, Crtam^{hi} CD4 T cells from C57BL/6 (CD45.2⁺) mice and Crtam⁻ CD4 T cells from congenic B6.SJL (CD45.1⁺) mice were purified by restricted FACS sorting. After resting for four days, Crtam^{hi} CD45.2⁺ and Crtam⁻ CD45.1⁺ T cells were labeled with CFSE and restimulated with plate-bound mAbs either as a single or mixed population for three more days. Cells were stained for CD45.2 and CFSE dilution examined by FACS analysis. Data shown here are representative of two independent experiments.

(G) Purified CD62L⁺CD4⁺ naive splenic T cells from C57BL/6 (CD45.2⁺) and congenic B6.SJL (CD45.1⁺) mice were stimulated with plate-bound anti-CD3 and anti-CD28 mAbs in T_{H}1 conditional media. Fourteen hours later, Crtam^{hi} CD4 T cells from C57BL/6 (CD45.2⁺) mice and Crtam⁻ CD4 T cells from congenic B6.SJL (CD45.1⁺) mice were purified by restricted FACS sorting. In order to enhance cell viability, cells were sorted at 20 psi sheath pressure and 2000 events per second and kept at 4° C. at all times using a BD FACSVantage Cell Sorter. Sorted cells were rested and cultured in T_{H}1 conditional media. 72 hours later, cells were re-stimulated with PMA plus Ionomycin in the presence of GolgiPlug (BD Biosciences) for 4 hours. Stimulated cells were stained with anti-CD45.2 mAbs and permeabilized for intracellular IFNγ staining Data shown here are representative of two independent experiments.

FIG. 14. Crtam interacts with Scrib PDZ3 and comparable Crtam surface expression in retroviral reconstituted Crtam^{-/-} CD4⁺ T cells.

(A) pCDNA4-Crtam-Flag was transfected into 293 cells and cell extracts were incubated with GST-Scrib-PDZ1, GST-Scrib-PDZ2, GST-Scrib-PDZ3, GST-Scrib-PDZ4 or GST-Erbb2ip (Erbin)-PDZ1. Immune complexes using anti-Flag M2-agarose beads were analyzed by immunoblotting with anti-GST (upper panel) or anti-Crtam (lower panel) antibodies.

(B) Crtam^{-/-} CD4⁺ T cells were retrovirally reconstituted with eGFP-IRES-Crtam, eGFP-IRES-Crtam (ΔICD) or eGFP-IRES-Crtam (ΔESIV). Surface expression of Crtam, Crtam (ΔICD) and eGFP-IRES-Crtam (ΔESIV) was examined by flow cytometry.

FIG. 15. The PDZ-binding motif of Crtam is required to control cell proliferation.

Cellular proliferation of retroviral reconstituted Crtam, Crtam (ΔICD), or Crtam (ΔESIV) in OT-II TCR⁺ Crtam^{-/-} CD4⁺ T cells in response to OVA peptide/APC stimulation. OT-II TCR⁺ Crtam^{+/-} and Crtam^{-/-} CD4⁺ T cells reconstituted with GFP served as controls. Data are representative of five independent experiments. Error bars indicate the standard deviation (SD). Statistical analysis was performed with a control using Dunnett's Method.

FIG. 16. Intracellular domain of Crtam controls T cell polarity and proliferation and the interaction of Cadm1 and Crtam can further enhance cytokine production of CD4 T cells

(A) Naive CD4⁺ T cells, either unstimulated or stimulated with anti-CD3 and anti-CD28 (10:2 mg/ml) mAbs for 14 hours, were stained with anti-Cadm1 rabbit polyclonal Ab (GNE) plus Talin or Crtam and analyzed by deconvolution microscopy.

(B) Structure of Crtam and Crtam(ΔECD) mutant.

(C) Naive Crtam^{-/-} CD4⁺ T cells were purified and activated with plate-bound anti-CD3 and anti-CD28 mAbs. Four days following stimulation, T cells were reconstituted with a retroviral vector encoding GFP or Flag-Crtam(ΔECD). Polarity of GFP, or Crtam(ΔECD) reconstituted Crtam^{-/-} CD4 T cells was examined at 8 h after TCR re-stimulation.

(D) Cellular proliferation of Crtam or Flag-Crtam(ΔECD) transfected Crtam^{-/-} CD4⁺ T cells was assessed by [³H]-thymidine incorporation. Crtam^{-/+} and Crtam^{-/-} CD4⁺ T cells transfected with GFP served as controls.

(E) Cytokines were analyzed by ELISA at 48 hours following TCR stimulation.

(F) Naive Crtam^{+/+} and Crtam^{-/-} CD4⁺ T cells were purified, activated with plate-bound mAbs plus plate-bound Cadm1(ECD)-Fc (1 μg/ml) or human IgG1 (1 μg/ml), and cytokines measured at 48 hours. N=2 independent experiments.

FIG. 17. Crtam is upregulated rapidly on CD8⁺ T cells following TCR activation and is required for optimal IFNγ, TNFα and IL22 production, but not for cytolytic activity.

(A) Naïve CD8⁺ T cells were purified from Crtam^{+/+} and Crtam^{-/-} mice, activated with plate-bound anti-CD3/28 mAbs and analyzed by flow cytometry at the indicated time points. Data shown are representative of two independent experiments.

(B) OT-I TCR⁻ mice were challenged with 20 μg OVA protein in 30 μl PBS by left footpad injection. The draining lymph node of the left foot and control lymph node of the right foot from immunized mice were analyzed for Crtam expression. Data is representative of three mice at each time point.

(C) Naïve (CD8⁺ CD62L⁺) and effector/memory (CD8⁺ CD62L⁻) CD8⁺ T cells were purified from Crtam^{+/+} and Crtam^{-/-} mice and stimulated with plate-bound anti-CD3/28 mAbs. Cytokine production was analyzed by ELISA 40 hr after stimulation. Data is representative of five independent experiments.

(D) CD8⁺ T cells from OT-I TCR⁺Crtam^{-/-} and OT-I TCR⁺Crtam^{+/+} mice were activated by plate-bound anti-CD3/28 mAbs for five days. CD8⁺ T cell blasts were incubated with 10 μM OVA₂₅₇₋₂₆₄ pulsed target cells (EL4) for 4 hr, and production of Granzyme B analyzed by ELISpot. Images are representative of two independent experiments with triplicate reactions. Quantitation of Granzyme B staining cells using a Leica M655 operating microscope is shown on the right.

(E) Day5 CD8⁺ T cell blasts from OT-I TCR⁺Crtam^{-/-} and OT-I TCR⁺Crtam^{+/+} mice were re-activated with peptide-pulsed EL4 in the presence of GolgiStop and anti-CD107 mAbs for 4 hr or in normal culture medium for 16 hr. Expression of CD107 at 4 hr and FasL at 16 hr on activated CD8⁻ T cell surface was analyzed by flow cytometry. Data shown are representative of two independent experiments. Statistical analysis was performed with a control using Dunnett's Method.

(F) Day5 CD8⁺ T cell blasts from OT-I TCR⁺Crtam^{-/-} and OT-I TCR⁺Crtam^{+/+} mice were re-activated with ⁵¹Cr-labeled EL4 cells pulsed with 10 μM OVA₂₅₇₋₂₆₄. ⁵¹Cr-release assays were performed 4 hr after incubation. Data is representative of two independent experiments with triplicate reactions.

FIG. 18. Crtam expression in CD8⁺ T cell is regulated upon activation.

OT-I TCR⁺ mice were challenged with 20 μg OVA protein in 30 μl PBS by footpad injection into the left foot. Draining lymph node of the left foot and control lymph node of the right foot from the immunized mice were analyzed for CD69 expression. Data is representative of three individual mice at each time points.

FIG. 19. Crtam is required for optimal IFNγ, TNFα and IL22 production.

Naïve and effector/memory CD8⁺ T cells were purified from OT-I TCR⁺ Crtam^{-/+} and Crtam^{-/-} mice and stimulated with irradiated APC pulsed with 10 μM OVA₂₅₇₋₂₆₄ for 40 hr. Cytokine production was analyzed by ELISA. Data is representative of two independent experiments.

FIG. 20. Normal TCR/CD3 expression on day 5 OT-I TCR⁺ Crtam^{-/-} CD8⁺ T cell blasts.

CD8⁺ T cells from OT-I TCR⁺ Crtam^{-/-} and OT-I TCR⁺ Crtam^{-/+} mice were activated by plate-bound anti-CD3/28 mAb for five days. Surface CD3 and TCR expression was examined by anti-CD3 and class I iTAg MHC tetramer (OVA peptides, Beckman Coulter) staining Data is representative of four independent experiments.

FIG. 21. Crtam maintains a late phase of T cell polarity and selective cytokine production through Scrib.

(A) Naïve CD8⁺ T cells were purified from Crtam^{+/+} and Crtam^{-/-} mice and stimulated with plate-bound anti-CD3/28 mAb for 16 hr. Cell extracts were immunoprecipitated with an anti-Scrib mAb (H-300, Santa Cruz Biotechnology, [SCB]). Immune complexes were analyzed by immunoblotting with antibodies against Crtam (17B2, GNE), Cdc42 (B-8, SCB), PKCζ (H-1, SCB), and Scrib (H-300).

(B) OT-I TCR⁻ Crtam^{+/+} and Crtam^{-/-} CD8⁺ T cells were activated with peptide/APC for 14 hr in a 37° C. incubator. Activated T cells were adhered onto Poly-D-Lysine-coated coverslips (BD BioCoat™) for 10 to 20 minutes at RT and were fixed, stained with anti-Crtam mAb (17B2, Genentech), or anti-CD3 mAb (BD Pharmingen), permeabilized with 0.2% Triton X-100, and stained with anti-Scrib (H-300), anti-Cdc42 (B-8), or anti-PKCζ (H-1) antibodies from Santa Cruz Biotechnology.

(C) Quantification of CD3 polarization in OT-I TCR⁺ Crtam^{+/+} and Crtam^{-/-} CD8⁺ T cells (n>200).

(D) Polarity of GFP control, Crtam or Crtam (ΔESIV) reconstituted Crtam^{-/-} CD8⁺ T cells was examined 14 hr after restimulation.

(E) Cytokine production of Crtam (blue), or Crtam (ΔESIV) (red) reconstituted Crtam^{-/-} CD8⁺ T cells was analyzed 48 hr after TCR stimulation. GFP reconstituted Crtam^{+/+} (white) or Crtam^{-/-} (green) T cells served as controls in D and E. Data is representative of three independent experiments. Slides were analyzed by deconvolution microscope (Delta Vision).

FIG. 22. Maintenance of the late stage of T cell polarity is disrupted in Crtam^{-/-} CD8⁺ T cells.

(A) CD8⁺ T cells were activated with plate-bound anti-CD3/28 mAbs for 16 hr in a 37° C. incubator. Activated T cells were flushed off the plate by gentle pipeting with media. Eluted cells were adhered onto Poly-D-Lysine-coated coverslips (BD BioCoat™) for 10 to 20 minutes at RT, and performed fixation and staining procedures.

(B) Quantification of CD3 polarization in Crtam^{+/+} and Crtam^{-/-} CD8⁺ T cells (n>200). Slides were analyzed by deconvolution microscope (Delta Vision).

FIG. 23. Cellular polarity and initial activation in Crtam^{-/-} CD8⁺ T cells are normal 30 minutes following TCR activation.

(A) 10 μl of anti-CD3/CD28 Abs coated Dynabeads were incubated with 1×10⁶ naïve CD8⁺ T cells from Crtam^{+/+} and Crtam^{-/-} mice for 30 min in 37° C. incubator, and cells were adhered onto Poly-D-Lysine-coated coverslips (BD BioCoat™) for 10 to 20 minutes at RT. Non-adherent cells were washed off with phosphate buffered saline (PBS) and cells were fixed with 4% paraformaldehyde in PBS, stained with anti-Crtam mAb (17B2, Genentech), or anti-CD3 mAb (BD Pharmingen), permeabilized with 0.2% Triton X-100, and stained with anti-Pericentrin (BD Pharmingen), anti-PKCθ (Cell Signaling), anti-Scrib (H-300), or anti-PKCζ (H-1) antibodies from Santa Cruz Biotechnology. Following the final wash, stained cells were mounted with ProLong Gold anti-fade reagent (Invitrogen). Images are representative of >50 cells for each experimental condition. Slides were analyzed by Leica SP5 confocal miroscope.

(B) Naïve CD8⁺ T cells from Crtam^{-/-} and Crtam^{-/+} mice were activated by plate-bound anti-CD3/28 mAbs. Surface CD25 and CD69 expression was examined 6 hr after activation. Data is representative of five independent experiments.

FIG. 24. Scrib and PKCζ are required to maintain the late phase of T cell polarity that, in turn, is required for IFNγ, TNFα and IL22 regulation.

(A-G) Day 5 CD8⁺ T cell blasts from Crtam^{+/+} mice were electroporated with control, Scrib (A, B, C panel 2, and D), or PKCζ (C, panel 3, E-G) siRNAs (QIAGEN) as described previously (Yeh et al., Cell 132(5):846-859(2008). Scrib and PKCζ expression were analyzed by Western blotting 12 hr after transfection (A and E), and T cells were restimulated with anti-CD3/CD28 mAbs for 8 hrs, cells fixed and stained for Crtam and Talin in addition of Scrib and/or PKCζ as denoted in the figure. Cytokine production by control, Scrib or PKCz siRNA transfected cells was analyzed by ELISA 48 hr after restimulation (D and G).

(H-J) pIRES-GFP or pIRES-Crtam(ΔICD):Scrib were electroporated into day 5 CD8⁺ T cell blasts from Crtam^{-/-} mice. Expression of Crtam(ΔICD):Scrib was confirmed by western blotting (H) and flow cytometry (FIG. 27). The grey arrow in this figure indicates endogenous Scrib protein and asterisk indicates the Crtam(ΔICD):Scrib chimera. Cellular polarity in Crtam(ΔICD):Scrib expressing cells was examined 8 hr after re-stimulation (I) and cytokine production by theses cells was analyzed 48 hr after restimulation by ELISA (J). Data is representative of two independent experiments. Images are representative of >50 cells examined for each staining.

FIG. 25. Talin polarization is not maintained in Scrib and PKCz knockdown CD8+ T cells. Quantification of Talin polarization in control, Scrib (A), or PKCζ (B) siRNA transfected Crtam^{+/-} CD8⁺ T cells 8hr after reactivation (n>50) as imaged in FIGS. 24B and 24F.

FIG. 26. Normal early stage T cell polarity and activation in Scrib and PKCζ knockdown CD8⁺ T cells.

(A) Control, Scrib, or PKCζ siRNA transfected Crtam^{+/+} CD8⁺ T cells were re-activated with anti-CD3/CD28 Abs coated Dynabeads for 30 minutes and fixed for anti-CD3 and anti-PKCθ staining (n>50).

(B) Control, Scrib, or PKCζ siRNA transfected Crtam^{+/-} CD8⁺ T cells were activated with plate-bound anti-CD3/CD28 Abs for 8 hr, and surface CD25 and CD69 expression was examined by flow cytometry.

FIG. 27. Expression of Crtam(ΔICD):Scrib chimera at the cell surface.

pIRES-GFP or pIRES-Crtam(ΔICD):Scrib were electroporated into day 5 CD8⁺ T cell blasts from Crtam^{-/-} mice. Expression of Crtam(ΔICD):Scrib was assessed by flow cytometry.

FIG. 28. Crtam mediated CD8 T cell responses is necessary for host resistance during L. monocytogenes infection.

(A) Rag2^{-/-} mice were intravenously injected with 1×10⁷ CD8⁺ Crtam^{-/-} or Crtam^{+/+} T cells a day before L. monocytogenes infection. Viability of mice was followed every day for two weeks.

(B) Surviving mice were euthanized at day 14 and the gross morphology of their spleens are shown.

(C) Bacterial burden of spleens from surviving mice were determined by colony counts on brain heart infusion (BHI) agar plates and quantified.

(D) Sera from day 5 infected mice were collected and analyzed by ELISA.

(E) 1×10⁷/ml splenocytes from day 5 infected mice were incubated with or without 1×10⁸ CFU/ml of heat-killed L. monocytogenes (HKLM). After 48 hr, cytokine production was analyzed by ELISA.

(F) Purified CD8⁺ T cells from day 5 infected mice were incubated with L. monocytogenes infected IC-21 target cells for 3 hr, and specific Granzyme B spots were counted by Leica M655 microscope. Data is representative of three independent experiments. Statistical analysis was performed with a control using Dunnett's Method.

FIG. 29. Examination of CD8⁺ T cells in reconstituted Rag2^{-/-} mice following L. monocytogenes infection.

(A) Rag2^{-/-} mice were intravenously injected with 1×10⁷ CD8⁺ Crtam^{-/-} or Crtam^{+/+} T cells a day before L. monocytogenes infection. Reconstituted CD8⁺ T cells in the blood were examined on the day of infection (Day 0), and on Day 14.

(B) CD8⁺ T cells in the spleen were examined on day 5 by flow cytometry. The percentage of CD8⁺ T cells in each individual mouse is shown in the right panels. Statistical analysis was performed with a control using Dunnett's Method.

FIG. 30. Human CRTAM amino acid sequence

(A) shows an amino acid sequence of human CRTAM with signal sequence (SEQ ID NO: 1). (B) shows an amino acid sequence of human CRTAM lacking the signal sequence (SEQ ID NO: 2).

FIG. 31. Mouse CRTAM amino acid sequence

(A) shows an amino acid sequence of mouse CRTAM (NM_{—}019465) with signal sequence (SEQ ID NO: 3). (B) shows an amino acid sequence of mouse CRTAM (NM_{—}019465) lacking the signal sequence (SEQ ID NO: 4).

FIG. 32. Human Necl2 (Cadm1) amino acid sequence

(A) shows an amino acid sequence of human Necl2 (Cadm1) (NM_{—}014333) with signal sequence (SEQ ID NO: 5). (B) shows an amino acid sequence of human Necl2 (Cadm1) (NM_{—}014333) lacking the signal sequence (SEQ ID NO: 6).

FIG. 33. Mouse Necl2 (Cadm1) amino acid sequence

(A) shows an amino acid sequence of mouse Necl2 (Cadm1) (NM_{—}207675) with signal sequence (SEQ ID NO: 7). (B) shows an amino acid sequence of mouse Necl2 (Cadm1) (NM_{—}207675) lacking the signal sequence (SEQ ID NO: 8).

FIG. 34. Anti-CRTAM antibody amino acid sequence

(A) shows an amino acid sequence of the light chain of a hamster-mouse chimeric anti-CRTAM antibody (17B2) (SEQ ID NO: 31). The variable domain is underlined (SEQ ID NO: 35); each of CDR1 (SEQ ID NO: 37), CDR2 (SEQ ID NO: 38), and CDR3 (SEQ ID NO: 39) are indicated by boxes. (B) shows an amino acid sequence of the heavy chain of a hamster-mouse chimeric anti-CRTAM antibody (17B2) (SEQ ID NO: 32). The variable domain is underlined (SEQ ID NO: 36); each of CDR1 (SEQ ID NO: 40), CDR2 (SEQ ID NO: 41), and CDR3 (SEQ ID NO: 42) are indicated by boxes.

FIG. 35. Anti-CRTAM antibody light chain nucleic acid sequence

The nucleic acid sequence of the light chain of a hamster-mouse chimeric anti-CRTAM antibody (17B2) is shown (SEQ ID NO: 33). The start and stop codons are underlined; the first codon of the mature light chain is indicated by the box.

FIG. 36. Anti-CRTAM antibody heavy chain nucleic acid sequence

The nucleic acid sequence of the heavy chain of a hamster-mouse chimeric anti-CRTAM antibody (17B2) is shown (SEQ ID NO: 34). The start and stop codons are underlined; the first codon of the mature light chain is indicated by the box.

### MODES FOR CARRYING OUT THE INVENTION

The invention provides methods, compositions, kits and articles of manufacture for diagnosing and treating a variety of disorders by modulating CRTAM in activated T cells. Details of these methods, compositions, kits and articles of manufacture are provided herein.

General Techniques

The practice of the invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988).

I. Definitions

A “disorder” or “disease”, as used herein in the context of CRTAM modulation, is any condition that would benefit from treatment with a CRTAM modulator and/or method of the invention. This includes chronic and acute disorders or diseases including those pathological conditions which predispose the mammal to the disorder or disease in question. Non-limiting examples of disorders or diseases to be treated herein include inflammatory (e.g., autoimmune), and other immunologic disorders/diseases.

An “autoimmune disease” herein is a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type I diabetes mellitus or insulin dependent diabetes mellitis); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia etc.

The invention also concerns active autoimmune diseases. An autoimmune disease that is active is one in which the subject's immune system is in an autoreactive state, that is, cells of the subject's own immune system are mobilized to attack the subject's own tissues and/or organs. A subject with an active autoimmune disease will generally exhibit corresponding symptoms of the disease. Mammalian subjects having an active autoimmune disease may be subject to a flare-up, which is a period of heightened disease activity or a return of corresponding symptoms. Flare-ups may occur in response to severe infection, allergic reactions, physical stress, emotional trauma, surgery, or environmental factors.

As described above, in treating inflammatory diseases (e.g. autoimmune diseases or autoimmune related conditions) described herein, a subject can be treated with a CRTAM modulator of the invention, in conjunction with a second therapeutic agent, such as an immunosuppressive agent (i.e., an anti-inflammatory agent), such as in a multi drug regimen. The CRTAM modulator can be administered concurrently, sequentially or alternating with the immunosuppressive agent. The immunosuppressive agent can be administered at the same or lesser dosages than as set forth in the art. The appropriate adjunct immunosuppressive agent will depend on many factors, including the type of disorder being treated as well as the patient's history.

“Immunosuppressive agent”, as used herein, refers to substances that act to suppress or mask the immune system and/or inflammatory response of a patient. Such agents would include substances that suppress cytokine production, down regulate or suppress self-antigen expression, or mask the MHC antigens. Examples of such agents include steroids such as glucocorticosteroids, e.g., prednisone, methylprednisolone, and dexamethasone; 2-amino-6-aryl-5-substituted pyrimidines (see U.S. Pat. No. 4,665,077), azathioprine (or cyclophosphamide, if there is an adverse reaction to azathioprine); bromocryptine; glutaraldehyde (which masks the MHC antigens, as described in U.S. Pat. No. 4,120,649); anti-idiotypic antibodies for MHC antigens and MHC fragments; cyclosporin A; cytokine or cytokine receptor antagonists including anti-interferon-γ, -β, or -α antibodies; anti-tumor necrosis factor-α antibodies; anti-tumor necrosis factor-β antibodies; anti-interleukin-2 antibodies and anti-IL2 receptor antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jun. 26, 1990); streptokinase; TGF-β; streptodornase; RNA or DNA from the host; FK506; RS-61443; deoxyspergualin; rapamycin; T-cell receptor (U.S. Pat. No. 5,114,721); T-cell receptor fragments (Ofiher et al., Science 251:430-432 (1991); WO 90/11294; and WO 91/01133); and T cell receptor antibodies (EP 340,109) such as T10B9.

The terms “Cytotoxic or Regulatory T cell Associated Molecule”, “class-I MHC restricted T-cell associated molecule” and “CRTAM” (in upper or lower case, or in any combination thereof), used interchangeably herein, encompass native sequence polypeptides, polypeptide variants and fragments of a native sequence polypeptide and polypeptide variants (which are further defined herein) that is capable of modulating activated T cell activity in a manner similar to wild type CRTAM. The CRTAM polypeptide described herein may be that which is isolated from a variety of sources, such as from human tissue types or from another source, or prepared by recombinant or synthetic methods. The terms “CRTAM”, “CRTAM polypeptide”, “CRTAM protein”, and “CRTAM molecule” also include variants of a CRTAM polypeptide as disclosed herein. A “CRTAM modulator” of the invention is a molecule that modulates the normal biological function/activity of CRTAM.

A “native sequence CRTAM polypeptide” comprises a polypeptide having the same amino acid sequence as the corresponding CRTAM polypeptide derived from nature. In one embodiment, a native sequence CRTAM polypeptide comprises the amino acid sequence of SEQ ID NO:1 (see FIG. 30A) or 2 (see FIG. 30B). Such native sequence CRTAM polypeptide can be isolated from nature or can be produced by recombinant or synthetic means. The terms “CRTAM polypeptide” and “CRTAM protein”, as used herein, specifically encompass naturally-occurring truncated or otherwise post-translationally modified forms of the specific CRTAM polypeptide, naturally-occurring variant forms (e.g., alternatively spliced forms) and naturally-occurring allelic variants of the polypeptide. As used herein, the terms “peptide” and “polypeptide” are used interchangeably, except that the term “peptide” generally refers to polypeptide comprising fewer than 200 contiguous amino acids.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR.sub.2 (“amidate”), P(O)R, P(O)OR′, CO or CH.sub.2 (“formacetal”), in which each R or R′ is independently H or substituted or unsubstituted alkyl (1-20 C.) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

The term “host cell” (or “recombinant host cell”), as used herein, is intended to refer to a cell that has been genetically altered, or is capable of being genetically altered by introduction of an exogenous polynucleotide, such as a recombinant plasmid or vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The CRTAM “extracellular domain” or “ECD” refers to a form of the CRTAM polypeptide, which is essentially free of the transmembrane and cytoplasmic domains of the respective full length molecules.

The term “CRTAM ligand,” refers to substances including, without limitation, a natural CRTAM ligand whether isolated and/or purified, synthetic, and/or recombinant, a homolog of a natural CRTAM ligand (e.g. from another mammal), antibodies, portions of such molecules, and other substances which bind CRTAM. The term CRTAM ligand encompasses substances which are inhibitors or promoters of CRTAM activity, as well as substances which bind but lack inhibitor or promoter activity. Examples of CRTAM ligands include, without limitation, nectin-like protein 2 (Necl2), also referred to as Cadm1.

The term “antagonist”, as used herein in relation to CRTAM function/activity, and the term “CRTAM antagonist”, are used in the broadest sense and includes any molecule that blocks the binding of a native sequence CRTAM polypeptide to a CRTAM ligand, and/or partially or fully blocks or neutralizes (collectively referred to as “inhibits”) or otherwise reduces a qualitative biological activity (as defined herein) of the native sequence CRTAM polypeptide. Suitable CRTAM antagonist molecules specifically include, without limitation, CRTAM modulators as described herein, for example modulators comprising blocking anti-CRTAM antibodies, including antibody fragments, other polypeptides, such as variants and fusions of the native sequence CRTAM polypeptides herein, peptide and non-peptide (organic) small molecules, inhibitory and antisense polynucleotide molecules. In one embodiment, CRTAM antagonists include anti-CRTAM antibodies, including antibody fragments, that specifically bind a native CRTAM polypeptide and are capable of blocking its binding to a CRTAM ligand.

A “CRTAM blocking antibody” or “blocking antibody” used in relation to CRTAM activity/fuction, refers to an antibody that is capable of blocking the binding of a native sequence CRTAM polypeptide to a CRTAM ligand, and/or partially or fully blocking or neutralizing (collectively referred to as “inhibiting”) or otherwise reducing a biological activity (as defined herein) of the native sequence CRTAM polypeptide. Blocking antibodies may be depleting antibodies as defined herein.

The term “CRTAM binding agent”, as used herein refers to a molecule, such as a protein, capable of binding CRTAM. In one embodiment, the binding agent binds CRTAM without interfering with CRTAM's ability to bind a CRTAM ligand. In one embodiment, a CRTAM binding agent is a CRTAM non-blocking antibody.

A “CRTAM non-blocking antibody” or “non-blocking antibody” used in relation to CRTAM function/activity, refers to an antibody that can bind a native sequence CRTAM polypeptide but does not interfere with or block the binding of a CRTAM ligand to the native sequence CRTAM polypeptide. However, upon binding, the CRTAM non-blocking antibody is capable of mediating depletion or deletion of the cell expressing the CRTAM molecule. In one embodiment, the non-blocking antibody binds to the CRTAM extracellular domain. Thus, in one embodiment, CRTAM non-blocking antibodies do not interfere with CRTAM ligand binding but are capable of inducing depletion of the T cells expressing CRTAM. Non-blocking antibodies may be depleting antibodies as defined herein.

“Depletion” as used herein refers to the removal, depletion, or deletion of a cell. In one embodiment, the depleted cell is part of a specific T cell population, for example, an activated CD4+ or CD8+ T cell population. In general, the depleted cells express a particular cell surface marker. The marker may be CD4 or CD8, singly or in combination with CRTAM. In one embodiment, the surface marker is CRTAM.

A “depleting antibody” as used herein refers to an antibody, the binding of which to a cell comprising its antigen target, results in an inhibition of antigen or cellular function or results in death of the cell. In one embodiment, a depleting antibody of the invention binds CRTAM, and may or may not block the binding of a CRTAM ligand to CRTAM. Thus, depleting antibodies specifically include blocking and non-blocking antibodies, as hereinabove defined. In certain embodiments, a depleting antibody may induce apoptosis or programmed cell death, e.g. of a T cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies). A depleting antibody that “induces cell death” is one which causes a viable cell to become nonviable. In one embodiment, the cell is a CRTAM⁺ cell. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the antibody, oligopeptide or other organic molecule is able to induce cell death, loss of membrane integrity as evaluated by uptake of propidium iodide (PI), trypan blue (see Moore et al. Cytotechnology 17:1-11 (1995)) or 7AAD can be assessed relative to untreated cells. In one embodiment, cell death-inducing antibodies are those which deplete a CRTAM⁺ cell.

In one embodiment, a depleting antibody of the invention binds CRTAM and comprises a toxin conjugate, wherein the toxin induces depletion of a cell binding the antibody conjugate. Depleting antibodies of the invention may also induce cell death through a conjugated toxin or cytotoxic agent.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof. Other cytotoxic agents are described below.

“Antibodies” (Abs) and “immunoglobulins” (Igs) refer to glycoproteins having similar structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The term “anti-CRTAM antibody” or “an antibody that binds to CRTAM” refers to an antibody that is capable of binding CRTAM with sufficient affinity such that the antibody is useful as a diagnostic and/or therapeutic agent in targeting CRTAM. In one embodiment, the extent of binding of an anti-CRTAM antibody to an unrelated, non-CRTAM protein is less than about 10% of the binding of the antibody to CRTAM as measured, e.g., by a radioimmunoassay (RIA). In certain embodiments, an antibody that binds to CRTAM has a dissociation constant (Kd) of ≦1 μM, ≦100 nM, ≦10 nM, ≦1 nM, or ≦0.1 nM. In certain embodiments, an anti-CRTAM antibody binds to an epitope of CRTAM that is conserved among CRTAM polypeptides from different species.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region. The Fc region of an immunoglobulin generally comprises two constant domains, a CH2 domain and a CH3 domain, and optionally comprises a CH4 domain. By “Fc region chain” herein is meant one of the two polypeptide chains of an Fc region.

“Fv” is a minimum antibody fragment which contains a complete antigen-binding site. In one embodiment, a two-chain Fv species consists of a dimer of one heavy- and one light-chain variable domain in tight, non-covalent association. Collectively, the six CDRs of an Fv confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO93/1161; Hudson et al. (2003) Nat. Med. 9:129-134; and Hollinger et al., Proc. Natl. Acad. Sci. USA 90: 6444-6448 (1993). Triabodies and tetrabodies are also described in Hudson et al. (2003) Nat. Med. 9:129-134.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256: 495 (1975); Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see, e.g., Marks et al., J. Mol. Biol., 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci USA, 90: 2551 (1993); Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

An “affinity matured” antibody is one with one or more alterations in one or more CDRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of HVR and/or framework residues is described by: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “small molecule” or “small organic molecule” is defined herein as an organic molecule having a molecular weight below about 500 Daltons.

A “CRTAM-binding oligopeptide” or an “oligopeptide that binds CRTAM” is an oligopeptide that is capable of binding CRTAM with sufficient affinity such that the oligopeptide is useful as a diagnostic and/or therapeutic agent in targeting CRTAM. In certain embodiments, the extent of binding of a CRTAM-binding oligopeptide to an unrelated, non-CRTAM protein is less than about 10% of the binding of the CRTAM-binding oligopeptide to CRTAM as measured, e.g., by a surface plasmon resonance assay. In certain embodiments, a CRTAM-binding oligopeptide has a dissociation constant (Kd) of ≦1 μM, ≦100 nM, ≦10 nM, ≦1 nM, or ≦0.1 nM.

A “CRTAM-binding organic molecule” or “an organic molecule that binds CRTAM” is an organic molecule other than an oligopeptide or antibody as defined herein that is capable of binding CRTAM with sufficient affinity such that the organic molecule is useful as a diagnostic and/or therapeutic agent in targeting CRTAM. In certain embodiments, the extent of binding of a CRTAM-binding organic molecule to an unrelated, non-CRTAM protein is less than about 10% of the binding of the CRTAM-binding organic molecule to CRTAM as measured, e.g., by a surface plasmon resonance assay. In certain embodiments, a CRTAM-binding organic molecule has a dissociation constant (Kd) of ≦1 μM, ≦100 nM, ≦10 nM, ≦1 nM, or ≦0.1 nM.

The dissociation constant (Kd) of any molecule that binds a target polypeptide may conveniently be measured using a surface plasmon resonance assay. Such assays may employ a BIAcore™-2000 or a BIAcore™-3000 (BIAcore, Inc., Piscataway, N.J.) at 25° C. with immobilized target polypeptide CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Target polypeptide is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of target polypeptide, 1 M ethanolamine is injected to block unreacted groups. For kinetics measurements, two-fold serial dilutions of the binding molecule (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneously fitting the association and dissociation sensorgrams. The equilibrium dissociation constant (Kd) is calculated as the ratio k_{off}/kₒₙ. See, e.g., Chen, Y., et al., (1999) J. Mol. Biol. 293:865-881. If the on-rate of an antibody exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stirred cuvette.

“Modulation”, used in relation to CRTAM function/activity, or “modulation of a CRTAM biological activity” as used herein refer, for example, to the modulation of a cellular event that (i) is part of an immune response involving a CRTAM⁺ cell, and (ii) is subject to modulation by a CRTAM modulator, including CRTAM antibodies (for example, an antagonist antibody). This modulation occurs after the initial stage of T cell activation, which generally includes a reorganization of the T cell cytoskeleton during the first hours of of cellular activation. During this initial reorganization, cytoskeletal regulators such as Was/Wasp, Vav1/Vav, Wasf2/WAVE2 and Hcls1/HS1 play a role in early phase T cell microtubule and actin reorganization. Other early stage events include, without limitation, one or more of the following: T cell activation by antigen presenting cells (APCs); formation of an immunological synapse; coordinated assembly of a signaling scaffold at the T cell antigen receptor (TCR) contact region; recruitment of Scrib and Dlg1 to the immunological synapse and lipid rafts; polarization of Scrib and Dlg1 away from CD3; the initial stages of actin polymerization; and cytoskeletal reorganization to generate second messengers following intercellular contact. A latter phase of T cell activation occurs subsequent to the initial T cell activation during which CRTAM expression is upregulated. Modulation of this latter phase by a CRTAM modulator may affect various T cell activation events including, without limitation, one or more of the following: cell proliferation; cycling or division; cell adhesion; development of T cell effector function; cytokine production; intracellular recruitment to the membrane of certain molecules including, without limitation, one or more of the following: the Scrib tumor-suppressor, PKCζ, and Cdc42; intracellular coordination of the assembly of a Cdc42 containing complex at the leading edge of T cells; later stage cytoskeletal reorganization, and T cell polarity. The CRTAM modulation may also affect certain CRTAM⁺ cell types including, without limitation, one or more of the following: CD4⁺ T cells, CD8⁺ T cells, NK cells, and NKT cells. Modulation by a CRTAM modulator, for example, includes an effect on cytokine production including, without limitation, one or more of the following cytokines: IFNγ, IL22 and/or IL17.

“CRTAM⁺ cell” as used herein refers to a cell having or expressing a native sequence CRTAM on its surface. In certain embodiments, a CRTAM⁺ cell is a T cell in a latter stage of T cell activation. CRTAM⁺ cells include, without limitation, a CD8⁺ T cell, a CD4⁺ T cell, an NK cell, or an NKT cell. In certain embodiments, a CRTAM⁺ cell is a cytokine producing cell, including without limitation IFNγ, IL22, and/or IL17. In one embodiment, a CRTAM⁺ cell is also CD4⁺. In one embodiment, a CRTAM⁺ cell is also CD8⁺. In one embodiment, a CRTAM⁺ cell is an activated CD4⁺ T cell.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol. 9:457-492 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

The terms “Fc receptor” or “FcR” are used to describe a receptor that binds to the Fc region of an antibody. In one embodiment, FcR is a native-sequence human FcR. In one embodiment, FcR is one that binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-492 (1991); Capel et al., Immunomethods 4:25-34 (1994); and de Haas et al., J. Lab. Clin. Med. 126:330-341 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Complement-dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

An “immune effector cell” refers to a cell capable of binding an antigen and mediating an immune response. These cells include, but are not limited to, T cells (e.g. CD4⁺, CD8⁺), B cells, monocytes, macrophages, NK cells and cytotoxic T lymphocytes (CTLs). In one embodiment, immune effector cells comprise activated cells. In one embodiment, a CRTAM⁺ cell is an immune effector cell.

A “naive” immune effector cell is an immune effector cell that has never been exposed to an antigen capable of activating that cell. Activation of naive immune effector cells requires both recognition of the peptide:MHC complex and the simultaneous delivery of a costimulatory signal by a professional APC in order to proliferate and differentiate into antigen-specific armed effector T cells.

The “pathology” of of a disorder, such as an autoimmune disease, includes all phenomena that compromise the well-being of the patient. This includes, without limitation, abnormal or uncontrollable cell growth (neutrophilic, eosinophilic, monocytic, lymphocytic cells), antibody production, auto-antibody production, complement production, interference with the normal functioning of neighboring cells, release of cytokines or other secretory products at abnormal levels, suppression or aggravation of any inflammatory or immunological response, infiltration of inflammatory cells (neutrophilic, eosinophilic, monocytic, lymphocytic) into cellular spaces, etc.

The term “autoreactive” as used herein refers to a condition of a cell in the immune system of a mammal having an autoimmune disease. Autoreactive T lymphocytes of the immune system are generally involved in the pathology of autoimmune diseases, as described herein. An autoreactive T cell may facilitate various events contributing to the initiation and perpetuation of an autoimmune response including, without limitation, one or more of: the induction of B cell autoantibody production, the activation of macrophages, the elevation of cytokine mRNA expression, and the elevation of secreted cytokine levels.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone, parathyroid hormone, thyroxine, insulin, proinsulin, relaxin, prorelaxin, glycoprotein hormones such as follicle stimulating hormone (FSH), thyroid stimulating hormone (TSH), and luteinizing hormone (LH), hepatic growth factor, fibroblast growth factor, prolactin, placental lactogen, tumor necrosis factor-α and -β, mullerian-inhibiting substance, mouse gonadotropin-associated peptide, inhibin, activin, vascular endothelial growth factor, integrin, thrombopoietin (TPO), nerve growth factors such as NGF-β, platelet-growth factor, transforming growth factors (TGFs) such as TGF-α and TGF-β, insulin-like growth factor-I and -II, erythropoietin (EPO), osteoinductive factors, interferons such as interferon-α, interferon-β, and interferon-γ; colony stimulating factors (CSFs) such as macrophage-CSF (M-CSF), granulocyte-macrophage-CSF (GM-CSF), and granulocyte-CSF (G-CSF), interleukins (ILs) such as IL1, IL1α, IL2, IL3, IL4, IL5, IL6, IL7, IL8, IL9, IL10, IL11, IL12, IL13, IL17, IL22, a tumor necrosis factor such as TNFα or TNFβ, and other polypeptide factors including LIF and kit ligand (KL). As used herein, the term cytokine includes proteins from natural sources or from recombinant cell culture and biologically active equivalents of the native sequence cytokines.

An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes nucleic acid molecules contained in cells that ordinarily express an encoded polypeptide where, for example, the nucleic acid molecule is in a chromosomal location different from that of natural cells.

An “isolated” polypeptide, including an isolated antibody, is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the polypeptide will be purified (1) to greater than 95% by weight of the compound as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated compound, e.g. antibody or other polypeptide, includes the compound in situ within recombinant cells since at least one component of the compound's natural environment will not be present. Ordinarily, however, isolated compound will be prepared by at least one purification step.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The phrase “substantially similar” or “substantially equivalent”, as used herein, denotes a sufficiently high degree of similarity between two numeric values such that one of skill in the art would consider the difference between the two values to be of little or no biological significance within the context of the biological characteristic measured by said values. The difference between said two values is preferably less than about 50%, preferably less than about 40%, preferably less than about 30%, preferably less than about 20%, preferably less than about 10%.

The term “agonist” is used in the broadest sense, and includes any molecule that partially or fully mimics or enhances a biological activity of a polypeptide, including, but not limited to, CRTAM and Necl2 (Cadm1), or that increases the transcription or translation of a nucleic acid encoding the polypeptide. Exemplary agonist molecules include, but are not limited to, agonist antibodies, polypeptide fragments, oligopeptides, organic molecules (including small molecules), and fusion polypeptides comprising a CRTAM-binding sequence.

The term “diagnosis” is used herein to refer to the identification or classification of a molecular or pathological state, disease or condition. For example, “diagnosis” may refer to identification of the presence, stage and/or extent, or type/subtype of an autoimmune disease, such as a particular type of lupus condition, e.g., SLE. “Diagnosis” may also refer to the classification of a particular sub-type of lupus, e.g., by tissue/organ involvement (e.g., lupus nephritis), by molecular features (e.g., a patient subpopulation characterized by genetic variation(s) in a particular gene or nucleic acid region.)

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, methods and compositions of the invention are useful in attempts to delay development of a disease or disorder.

The term “prevention”, “inhibition”, and “prophylaxis”, as used in the context of the invention includes the situation wherein the occurrence of a pathological state, disease or condition is completely or partially blocked, the onset of a pathological state, disease or condition is partially or completely delayed, or the stimulation of an existing pathological state, disease or condition is partially or completely reversed. Whereas it is foreseen that an existing pathological state, disease or condition may be completely or partially reversed, this is not a requirement under this definition.

“Ameliorate” as used herein, is defined herein as to make better or improve.

An “effective amount” refers to an amount effective, at dosages and for periods of time necessary, to achieve the desired therapeutic or prophylactic result. A “therapeutically effective amount” of a therapeutic agent may vary according to factors such as the disease state, age, sex, and weight of the individual, and the ability of the antibody to elicit a desired response in the individual. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

An “individual,” “subject” or “patient” is a vertebrate. In certain embodiments, the vertebrate is a mammal. Mammals include, but are not limited to, farm animals (such as cows), sport animals, pets (such as cats, dogs, and horses), primates (including human and non-human primates), and rodents (e.g., mice and rats). In certain embodiments, a mammal is a human.

The term “test sample” refers to a sample from a subject suspected of having a pathological state, disease or condition (such as an autoimmune disease). The test sample may originate from various sources in the subject including, without limitation, blood, serum, bone marrow, etc.

The term “control” refers a negative control in which a negative result is expected to help correlate a positive result in the test sample. Controls that are suitable for the invention include, without limitation, a sample known to be devoid of activated T cells expressing CRTAM, a sample known to be devoid of activated CD4+ T cells expressing CRTAM, and a sample obtained from a subject known not to have a relevant pathological state, disease or condition. In addition, the control may be a sample containing normal cells that have the same origin as cells contained in the test sample. Those of skill in the art will appreciate other controls suitable for use in the invention.

A “medicament” is an active drug to treat a pathological state, disease, and/or condition. In one embodiment, the pathological state, disease, and/or condition is an autoimmune disorder as set forth herein, or its symptoms or side effects.

II. Compositions and Methods of the Invention

The CRTAM modulators of the invention, including anti-CRTAM antibodies and fragments thereof, can be used to modulate a biological activity involving a CRTAM⁺ cell. In one embodiment, the methods may be used to treat or prevent a disease, such as an autoimmune disease, by administering an effective amount of a CRTAM modulator to a subject in need. Thus, the CRTAM modulators (e.g., CRTAM antagonists) herein can be used to treat or prevent such diseases that are related to a biological activity involving a CRTAM⁺ cell that is subject to modulation by the CRTAM modulator. In one embodiment, the CRTAM modulator comprises an antagonist anti-CRTAM antibody or a fragment thereof, which may act as a blocking antibody.

In another embodiment, a CRTAM modulator comprises a blocking antibody or fragment thereof, which may induce depletion of a CRTAM⁺ cell. Antibodies against cell-surface molecules have been shown to be effective in depleting or removing specific lymphocyte subsets or to inhibit cell function. For example, the use of monoclonal antibodies against the cell surface receptor CD45RB is believed to lead to functional and/or actual deletion of T cell clones expressing a receptor having the corresponding CD45RB antigen (Lazarovits, et al. U.S. Pat. No. 7,160,987). Such antibodies seem to be capable of selectively inhibiting the inflammatory and cytotoxic T-cell mediated immune response without destroying the pool of memory T-cells. These types of antibodies have the potential to act on a particular T-cell population rather than having an overall immunosuppressive effect, and to confer a long term tolerance to a particular antigen when they are administered contemporaneously with exposure to the antigen, such as just before and after a tissue or organ transplant, or during an acute phase of an autoimmune disease. In one embodiment, the antibodies of the invention are capable of acting on a particular T-cell sub-population rather than having an overall immunosuppressive effect. In one embodiment, the T-cell sub-population comprises a CRTAM+ T cell sub-population, e.g., an activated CD4+CRTAM+ T cell sub-population.

CRTAM modulators of the invention, including anti-CRTAM antibodies and fragments thereof, can be used to deplete a CRTAM⁺ cell that is subject to depletion by such CRTAM modulators. In one embodiment, the CRTAM⁻ cell is a cytotoxic T cell or an immune effector cell. In one embodiment, the methods may be used to treat or prevent a disease, such as an autoimmune disease, by administering an effective amount of a CRTAM modulator to a subject in need. Thus, the CRTAM modulators herein can be used to treat or prevent such diseases that may be ameliorated by the depletion of a CRTAM⁺ cell. The CRTAM modulator may comprise an anti-CRTAM antibody or a fragment thereof, which may act as a non-blocking antibody.

In one embodiment of the methods herein, no other medicament than the CRTAM modulator, such as an anti-CRTAM antibody, is administered to the subject to treat or prevent the disease. In one embodiment, one may administer to the subject along with the CRTAM modulator an effective amount of a second medicament (where the CRTAM modulator (e.g., the anti-CRTAM antibody) is a first medicament). The second medicament may be one or more medicaments, and include, for example, an immunosuppressive agent, cytokine antagonist such as a cytokine antibody, growth factor, hormone, integrin, integrin antagonist or antibody, or any combination thereof. The type of such second medicament depends on various factors, including the type of immune disease, the severity of the immune disease, the condition and age of the subject, the type and dose of first medicament employed, etc.

CRTAM modulators of the invention are particularly useful in autoimmune disease diagnostic and prognostic assays, and imaging methodologies. In one embodiment, the assays are performed using an anti-CRTAM antibody or a fragment thereof. The invention also provides various immunological assays useful for the detection and quantification of CRTAM proteins. These assays are performed within various immunological assay formats well known in the art, including but not limited to various types of radioimmunoassays, enzyme-linked immunosorbent assays (ELISA), enzyme-linked immunofluorescent assays (ELIFA), and the like. In addition, immunological imaging methods capable of detecting autoimmune disease characterized by CRTAM expression are also provided by the invention, including but not limited to radioscintigraphic imaging methods using, for example, labeled CRTAM antibodies. Such assays are clinically useful in the detection, monitoring, and prognosis of autoimmune diseases characterized by CRTAM expression.

Another aspect of the invention relates to methods for identifying a cell that expresses CRTAM. The expression profile of CRTAM makes it a diagnostic marker for disorders such as autoimmune diseases. Accordingly, the status of CRTAM expression provides information useful for predicting a variety of factors including susceptibility to advanced stages of disease, rate of progression, and/or sudden and severe onset of symptoms in an active disease (e.g., an active autoimmune disease), i.e. flare-ups.

In one embodiment, the invention provides methods of detecting a disorder, such as an autoimmune disease. For example, a test sample from a subject and a control are each contacted with, for example, an anti-CRTAM antibody or a fragment thereof. In one embodiment, the test sample contains T cells, for example, activated T cells, having certain cell surface markers, including without limitation, one or more of the following: CRTAM⁺, CD4⁺, and CD8⁺. In one embodiment, the test sample contains T cells that are activated CRTAM⁺, CD4⁺ T cells. The amount of CRTAM⁺ cells is measured and a higher relative amount of cells in the test sample as compared to the control is indicative of a disease (such as an autoimmune disease) in the subject from which the test sample was obtained. The methods of detection may further comprise the steps of obtaining a second test sample containing T cells from the subject, contacting the second test sample and the original test sample with an anti-CRTAM antibody, detecting a higher relative amount of CRTAM⁺ cells in the second test sample as compared to the original test sample, where the higher relative amount is indicative of a flare-up in the disease (such as an autoimmune disease) of the subject from which the test samples were obtained.

In another embodiment, an autoimmune disease detected by the methods of the invention is an active autoimmune disease. In some embodiments, the methods are employed to detect a flare-up in the active autoimmune disease. Following initial detection of an autoimmune disease, additional test samples may be obtained from the subject found to have an autoimmune disease. The additional sample may be obtained hours, days, weeks, or months after the initial sample was taken. Those of skill in the art will appreciate the appropriate schedule for obtaining such additional samples, which may include second, third, fourth, fifth, sixth, etc. test samples. The intial test sample and the additional sample(s) (and alternately a control as described herein) are contacted with, for example, an anti-CRTAM antibody. The amount of CRTAM⁺ cells is measured and a higher relative amount of cells in the additional test sample as compared to the initial test sample is indicative of a flare-up in the active autoimmune disease in the subject from which the test sample was obtained.

The invention provides assays for detecting the presence of CRTAM in a tissue or other biological sample such as serum, semen, bone, prostate, urine, cell preparations, and the like. Methods for detecting CRTAM are also well known and include, for example, immunoprecipitation, immunohistochemical analysis, Western blot analysis, molecular binding assays, ELISA, ELIFA and the like. For example, a method of detecting the presence of a CRTAM protein in a biological sample comprises first contacting the sample with, for example, an anti-CRTAM antibody, a CRTAM-reactive fragment thereof, or a recombinant protein containing an antigen-binding region of an anti-CRTAM antibody; and then detecting the binding of a CRTAM protein in the sample.

In another aspect, the invention provides methods of providing isolated populations of activated CD4+ T cells. The methods of isolating activated CD4+ T cells include the step of contacting a biological sample that contains a mixed population T cells from a subject with a CRTAM binding molecule (e.g., a CRTAM modulator as described herein), such as an anti-CRTAM antibody. The sample may be obtained from the subject by methods known in the art. The sample containing a mixture of T cells is contacted with a molecule that binds specifically to CRTAM. In one embodiment, the molecule is an anti-CRTAM antibody or fragment thereof. Any cells expressing CRTAM will bind to the CRTAM binding molecule, thereby distinguishing them from cells not expressing CRTAM and permitting separation and isolation. Methods of separating the binding molecules from the cells to which they are bound are well known in the art. For example, antibodies may be separated from cells by a short exposure to a low pH solution, or with a protease such as chymotrypsin. Alternatively, the isolation of populations of CRTAM+ cells may be achieved through the use of conjugated labels that expedite identification and separation. Examples of such labels include magnetic beads; biotin, which may be identified or separated by means of its affinity to avidin or streptavidin; fluorochromes, which may be identified or separated by means of a fluorescence-activated cell sorter (FACS, see below), and the like. Any technique may be used for isolation as long as the technique does not unduly harm the CRTAM+ cells. Many such methods are known in the art.

In one embodiment, a CRTAM binding molecule is attached to a solid support. Some suitable solid supports include nitrocellulose, agarose beads, polystyrene beads, hollow fiber membranes, magnetic beads, and plastic petri dishes. For example, the binding molecule can be covalently linked to Pharmacia Sepharose 6 MB macro beads. The exact conditions and duration of incubation for the solid phase-linked binding molecules with the crude cell mixture will depend upon several factors specific to the system employed, as is well known in the art.

Cells that are bound to the binding molecule are removed from the cell suspension by physically separating the solid support from the remaining cell suspension. For example, the unbound cells may be eluted or washed away with physiologic buffer after allowing sufficient time for the solid support to bind the CRTAM+ cells.

The bound cells are separated from the solid phase by any appropriate method, depending mainly upon the nature of the solid phase and the binding molecule. For example, bound cells can be eluted from a plastic petri dish by vigorous agitation. Alternatively, bound cells can be eluted by enzymatically “nicking” or digesting an enzyme-sensitive “spacer” sequence between the solid phase and an antibody. Suitable spacer sequences bound to agarose beads are commercially available from, for example, Pharmacia.

The eluted, enriched fraction of cells may then be washed with a buffer by centrifugation and preserved in a viable state at low temperatures for later use according to methods known in the art.

III. Populations of Activated CD4+ T Cells

In another aspect, the invention relates to an isolated population of activated CD4+ T cells, where, for example, the majority or substantially all of the T cells in such isolated population are in late phase activation. In one embodiment, the isolated populations are characterized by (1) expression of CRTAM, and (2) an elevated level of cytokine mRNA expression relative to CD4+ activated T cells not expressing CRTAM. In addition, the cytokine mRNA overexpressing cells may also exhibit elevated cytokine secretion levels. In other embodiments, the cytokines are preferably IFNγ, IL22, and/or IL17.

In another embodiment, the isolated population is purified such that it contains a higher proportion of activated CD4+ T cells than the crude population of cells from which the activated CD4+ T cells are isolated. The purified population of activated CD4+ T cells may be isolated by contacting a crude mixture of cells containing a population of T cells that express an antigen characteristic of activated T cells with a molecule that binds specifically to the extracellular portion of the antigen. Such a technique is known as positive selection. The binding of the activated T cells to the molecule permits the T cells to be sufficiently distinguished from contaminating cells that do not express the antigen to permit isolating the T cells from the contaminating cells. Generally and preferably, the antigen comprises CRTAM.

A CRTAM binding molecule used to separate activated CD4+ T cells from the contaminating cells can be any molecule that binds specifically to the CRTAM expressed on the activated CD4+ cell to be isolated. The molecule can be, for example, an antibody or fragment thereof as described herein. In one embodiment, the molecule is a CRTAM blocking antibody or CRTAM non-blocking antibody.

In one embodiment, the isolated population of activated CD4+ T cells provided by the invention contains one or more CRTAM⁺ cells as defined herein.

IV. Modulator Antibodies

In one embodiment, the invention provides CRTAM modulator antibodies which may find use herein as therapeutic and/or diagnostic agents. Exemplary antibodies include polyclonal, monoclonal, humanized, multispecific, and heteroconjugate antibodies. Aspects of generating, identifying, characterizing, modifying and producing antibodies are set forth below, and are well established in the art, e.g., as described in US Pat. Appl. Pub. No. 2005/0042216 from paragraphs 522 through 563, 604 through 608, and 617 through 688.

(i) Antigen Preparation

Soluble antigens or fragments thereof, optionally conjugated to other molecules, can be used as immunogens for generating antibodies. For transmembrane molecules, such as receptors, fragments of these (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

(ii) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCI₂, or R₁N═C═NR, where R and R₁ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

Monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp.59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al, Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subloned by limiting dilution procedures and grown by standard methods (Goding, MonoclonalAntibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described in McCafferty et al., Nature, 348:552-554 (1990).

Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

In one embodiment, the invention provides methods of making monoclonal antibodies to CRTAM for the inhibition of T cell activation.

(iv) Humanized and Human Antibodies

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad Sci. USA, 89:4285 (1992); Presta et al., J. Immnol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Natl. Acad. Sci. USA, 90:2551 (1993); Jakobovits et al., Nature, 362:255-258 (1993); Bruggermann et al., Year in Immunol., 7:33 (1993); and Duchosal et al. Nature 355:258 (1992). Human antibodies can also be derived from phage-display libraries (Hoogenboom et al, J. Mol. Biol., 227:381 (1991); Marks et al, J. Mol. Biol., 222:581-597 (1991); Vaughan et al. Nature Biotech 14:309 (1996)). Generation of human antibodies from antibody phage display libraries is further described below.

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). In another embodiment as described in the example below, the F(ab′)₂ is formed using the leucine zipper GCN4 to promote assembly of the F(ab′)₂ molecule. According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

(vi) Multispecific Antibodies

Multispecific antibodies have binding specificities for at least two different epitopes, where the epitopes are usually from different antigens. While such molecules normally will only bind two different epitopes (i.e. bispecific antibodies, BsAbs), antibodies with additional specificities such as trispecific antibodies are encompassed by this expression when used herein. Examples of BsAbs include those with one arm directed against CRTAM and another arm directed against another protein playing a role in immune complex clearance, such as a macrophage receptor selected from the group of CR1, CR2, CR3, and CR4.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991). According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. A preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′-SH fragments can also be directly recovered from E. coli, and can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al, J. Immunol, 152:5368 (1994).

Antibodies with more than two specificities are contemplated. For example, trispecific antibodies can be prepared. Tuft et al. J. Immunol. 147: 60 (1991). In addition, multivalent (e.g. bivalent) antibodies with more than one binding specificity to the same antigen are also within the scope herein.

(vii) Effector Function Engineering

It may be desirable to modify the antibody of the invention with respect to effector function, so as to enhance the effectiveness of the antibody. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctonal cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al Anti-Cancer Drug Design 3:219-230 (1989).

(viii) Antibody-Salvage Receptor Binding Epitope Fusions

In certain embodiments of the invention, it may be desirable to use an antibody fragment, rather than an intact antibody, to increase tumor penetration, for example. In this case, it may be desirable to modify the antibody fragment in order to increase its serum half life. This may be achieved, for example, by incorporation of a salvage receptor binding epitope into the antibody fragment (e.g. by mutation of the appropriate region in the antibody fragment or by incorporating the epitope into a peptide tag that is then fused to the antibody fragment at either end or in the middle, e.g., by DNA or peptide synthesis).

The salvage receptor binding epitope preferably constitutes a region wherein any one or more amino acid residues from one or two loops of a Fc domain are transferred to an analogous position of the antibody fragment. Even more preferably, three or more residues from one or two loops of the Fc domain are transferred. Still more preferred, the epitope is taken from the CH2 domain of the Fc region (e.g., of an IgG) and transferred to the CH1, CH3, or V.sub.H region, or more than one such region, of the antibody. Alternatively, the epitope is taken from the CH2 domain of the Fc region and transferred to the CL region or VL region, or both, of the antibody fragment.

(ix) Other Covalent Modifications of Antibodies

Covalent modifications of antibodies are included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues. Examples of covalent modifications are described in U.S. Pat. No. 5,534,615, specifically incorporated herein by reference. A preferred type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

(x) Generation of Antibodies from Synthetic Antibody Phage Libraries

In one embodiment, the invention provides a method for generating and selecting novel antibodies using a unique phage display approach. The approach involves generation of synthetic antibody phage libraries based on single framework template, design of sufficient diversities within variable domains, display of polypeptides having the diversified variable domains, selection of candidate antibodies with high affinity to target the antigen, and isolation of the selected antibodies.

Details of the phage display methods can be found, for example, WO03/102157 published Dec. 11, 2003, the entire disclosure of which is expressly incorporated herein by reference.

In one aspect, the antibody libraries used in the invention can be generated by mutating the solvent accessible and/or highly diverse positions in at least one CDR of an antibody variable domain. Some or all of the CDRs can be mutated using the methods provided herein. In some embodiments, it may be preferable to generate diverse antibody libraries by mutating positions in CDRH1, CDRH2 and CDRH3 to form a single library or by mutating positions in CDRL3 and CDRH3 to form a single library or by mutating positions in CDRL3 and CDRH1, CDRH2 and CDRH3 to form a single library.

A library of antibody variable domains can be generated, for example, having mutations in the solvent accessible and/or highly diverse positions of CDRH1, CDRH2 and/or CDRH3. Another library can be generated having mutations in CDRL1, CDRL2 and/or CDRL3. These libraries can also be used in conjunction with each other to generate binders of desired affinities. For example, after one or more rounds of selection of heavy chain libraries for binding to a target antigen, a light chain library can be replaced into the population of heavy chain binders for further rounds of selection to increase the affinity of the binders.

Preferably, a library is created by substitution of original amino acids with variant amino acids in the CDRH3 region of the variable region of the heavy chain sequence. The resulting library can contain a plurality of antibody sequences, wherein the sequence diversity is primarily in the CDRH3 region of the heavy chain sequence.

In one aspect, the library is created in the context of the humanized antibody 4D5 sequence, or the sequence of the framework amino acids of the humanized antibody 4D5 sequence. Preferably, the library is created by substitution of at least residues 95-100a of the heavy chain with amino acids encoded by the DVK codon set, wherein the DVK codon set is used to encode a set of variant amino acids for every one of these positions. An example of an oligonucleotide set that is useful for creating these substitutions comprises the sequence (DVK)₇. In some embodiments, a library is created by substitution of residues 95-100a with amino acids encoded by both DVK and NNK codon sets. An example of an oligonucleotide set that is useful for creating these substitutions comprises the sequence (DVK)₆ (NNK). In another embodiment, a library is created by substitution of at least residues 95-100a with amino acids encoded by both DVK and NNK codon sets. An example of an oligonucleotide set that is useful for creating these substitutions comprises the sequence (DVK)₅ (NNK). Another example of an oligonucleotide set that is useful for creating these substitutions comprises the sequence (NNK)₆. Other examples of suitable oligonucleotide sequences can be determined by one skilled in the art according to the criteria described herein.

In another embodiment, different CDRH3 designs are utilized to isolate high affinity binders and to isolate binders for a variety of epitopes. The range of lengths of CDRH3 generated in this library is 11 to 13 amino acids, although lengths different from this can also be generated. H3 diversity can be expanded by using NNK, DVK and NVK codon sets, as well as more limited diversity at N and/or C-terminal.

Diversity can also be generated in CDRH1 and CDRH2. The designs of CDR-H1 and H2 diversities follow the strategy of targeting to mimic natural antibodies repertoire as described with modification that focus the diversity more closely matched to the natural diversity than previous design.

For diversity in CDRH3, multiple libraries can be constructed separately with different lengths of H3 and then combined to select for binders to target antigens. The multiple libraries can be pooled and sorted using solid support selection and solution sorting methods as described previously and herein below. Multiple sorting strategies may be employed. For example, one variation involves sorting on target bound to a solid, followed by sorting for a tag that may be present on the fusion polypeptide (eg., anti-gD tag) and followed by another sort on target bound to solid. Alternatively, the libraries can be sorted first on target bound to a solid surface, the eluted binders are then sorted using solution phase binding with decreasing concentrations of target antigen. Utilizing combinations of different sorting methods provides for minimization of selection of only highly expressed sequences and provides for selection of a number of different high affinity clones.

High affinity binders for the target antigen can be isolated from the libraries. Limiting diversity in the H1/H2 region decreases degeneracy about 10⁴ to 10⁵ fold and allowing more H3 diversity provides for more high affinity binders. Utilizing libraries with different types of diversity in CDRH3 (eg. utilizing DVK or NVT) provides for isolation of binders that may bind to different epitopes of a target antigen.

Of the binders isolated from the pooled libraries as described above, it has been discovered that affinity may be further improved by providing limited diversity in the light chain. Light chain diversity is generated in this embodiment as follows in CDRL1: amino acid position 28 is encoded by RDT; amino acid position 29 is encoded by RKT; amino acid position 30 is encoded by RVW; amino acid position 31 is encoded by ANW; amino acid position 32 is encoded by THT; optionally, amino acid position 33 is encoded by CTG; in CDRL2: amino acid position 50 is encoded by KBG; amino acid position 53 is encoded by AVC; and optionally, amino acid position 55 is encoded by GMA; in CDRL3: amino acid position 91 is encoded by TMT or SRT or both; amino acid position 92 is encoded by DMC; amino acid position 93 is encoded by RVT; amino acid position 94 is encoded by NHT; and amino acid position 96 is encoded by TWT or YKG or both.

In another embodiment, a library or libraries with diversity in CDRH1, CDRH2 and CDRH3 regions is generated. In this embodiment, diversity in CDRH3 is generated using a variety of lengths of H3 regions and using primarily codon sets XYZ and NNK or NNS. Libraries can be formed using individual oligonucleotides and pooled or oligonucleotides can be pooled to form a subset of libraries. The libraries of this embodiment can be sorted against target bound to solid. Clones isolated from multiple sorts can be screened for specificity and affinity using ELISA assays. For specificity, the clones can be screened against the desired target antigens as well as other nontarget antigens. Those binders to the target antigen can then be screened for affinity in solution binding competition ELISA assay or spot competition assay. High affinity binders can be isolated from the library utilizing XYZ codon sets prepared as described above. These binders can be readily produced as antibodies or antigen binding fragments in high yield in cell culture.

In some embodiments, it may be desirable to generate libraries with a greater diversity in lengths of CDRH3 region. For example, it may be desirable to generate libraries with CDRH3 regions ranging from about 7 to 19 amino acids.

High affinity binders isolated from the libraries of these embodiments are readily produced in bacterial and eukaryotic cell culture in high yield. The vectors can be designed to readily remove sequences such as gD tags, viral coat protein component sequence, and/or to add in constant region sequences to provide for production of full length antibodies or antigen binding fragments in high yield.

A library with mutations in CDRH3 can be combined with a library containing variant versions of other CDRs, for example CDRL1, CDRL2, CDRL3, CDRH1 and/or CDRH2. Thus, for example, in one embodiment, a CDRH3 library is combined with a CDRL3 library created in the context of the humanized 4D5 antibody sequence with variant amino acids at positions 28, 29, 30,31, and/or 32 using predetermined codon sets. In another embodiment, a library with mutations to the CDRH3 can be combined with a library comprising variant CDRH1 and/or CDRH2 heavy chain variable domains. In one embodiment, the CDRH1 library is created with the humanized antibody 4D5 sequence with variant amino acids at positions 28, 30, 31, 32 and 33. A CDRH2 library may be created with the sequence of humanized antibody 4D5 with variant amino acids at positions 50, 52, 53, 54, 56 and 58 using the predetermined codon sets.

(xi) Antibody Mutants

The novel antibodies generated from phage libraries can be further modified to generate antibody mutants with improved physical, chemical and or biological properties over the parent antibody. Where the assay used is a biological activity assay, the antibody mutant preferably has a biological activity in the assay of choice which is at least about 10 fold better, preferably at least about 20 fold better, more preferably at least about 50 fold better, and sometimes at least about 100 fold or 200 fold better, than the biological activity of the parent antibody in that assay. For example, an anti-CRTAM antibody mutant preferably has a binding affinity for CRTAM which is at least about 10 fold stronger, preferably at least about 20 fold stronger, more preferably at least about 50 fold stronger, and sometimes at least about 100 fold or 200 fold stronger, than the binding affinity of the parent antibody.

To generate the antibody mutant, one or more amino acid alterations (e.g. substitutions) are introduced in one or more of the hypervariable regions of the parent antibody. Alternatively, or in addition, one or more alterations (e.g. substitutions) of framework region residues may be introduced in the parent antibody where these result in an improvement in the binding affinity of the antibody mutant for the antigen from the second mammalian species. Examples of framework region residues to modify include those which non-covalently bind antigen directly (Amit et al. (1986) Science 233:747-753); interact with/effect the conformation of a CDR (Chothia et al. (1987) J. Mol. Biol. 196:901-917); and/or participate in the V_{L}-V_{H} interface (EP 239 400B1). In certain embodiments, modification of one or more of such framework region residues results in an enhancement of the binding affinity of the antibody for the antigen from the second mammalian species. For example, from about one to about five framework residues may be altered in this embodiment of the invention. Sometimes, this may be sufficient to yield an antibody mutant suitable for use in preclinical trials, even where none of the hypervariable region residues have been altered. Normally, however, the antibody mutant will comprise additional hypervariable region alteration(s).

The hypervariable region residues which are altered may be changed randomly, especially where the starting binding affinity of the parent antibody is such that such randomly produced antibody mutants can be readily screened.

One useful procedure for generating such antibody mutants is called “alanine scanning mutagenesis” (Cunningham and Wells (1989) Science 244:1081-1085). Here, one or more of the hypervariable region residue(s) are replaced by alanine or polyalanine residue(s) to affect the interaction of the amino acids with the antigen from the second mammalian species. Those hypervariable region residue(s) demonstrating functional sensitivity to the substitutions then are refined by introducing further or other mutations at or for the sites of substitution. Thus, while the site for introducing an amino acid sequence variation is predetermined, the nature of the mutation per se need not be predetermined. The ala-mutants produced this way are screened for their biological activity as described herein.

Normally one would start with a conservative substitution such as those shown below under the heading of “preferred substitutions”. If such substitutions result in a change in biological activity (e.g. binding affinity), then more substantial changes, denominated “exemplary substitutions” in the following table, or as further described below in reference to amino acid classes, are introduced and the products screened.

Exemplary/Preferred Substitutions:

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; met; | ile |
| | ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

Even more substantial modifications in the antibodies biological properties are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr, asn, gln;
(3) acidic: asp, glu;
(4) basic: his, lys, arg;
(5) residues that influence chain orientation: gly, pro; and
(6) aromatic: trp, tyr, phe.

Non-conservative substitutions will entail exchanging a member of one of these classes for another class.

In another embodiment, the sites selected for modification are affinity matured using phage display (see above).

Nucleic acid molecules encoding amino acid sequence mutants are prepared by a variety of methods known in the art. These methods include, but are not limited to, oligonucleotide-mediated (or site-directed) mutagenesis, PCR mutagenesis, and cassette mutagenesis of an earlier prepared mutant or a non-mutant version of the parent antibody. The preferred method for making mutants is site directed mutagenesis (see, e.g., Kunkel (1985) Proc. Natl. Acad. Sci. USA 82:488).

In certain embodiments, the antibody mutant will only have a single hypervariable region residue substituted. In other embodiments, two or more of the hypervariable region residues of the parent antibody will have been substituted, e.g. from about two to about ten hypervariable region substitutions.

Ordinarily, the antibody mutant with improved biological properties will have an amino acid sequence having at least 75% amino acid sequence identity or similarity with the amino acid sequence of either the heavy or light chain variable domain of the parent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e same residue) or similar (i.e. amino acid residue from the same group based on common side-chain properties, see above) with the parent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

Following production of the antibody mutant, the biological activity of that molecule relative to the parent antibody is determined. As noted above, this may involve determining the binding affinity and/or other biological activities of the antibody. In a preferred embodiment of the invention, a panel of antibody mutants is prepared and screened for binding affinity for the antigen or a fragment thereof. One or more of the antibody mutants selected from this initial screen are optionally subjected to one or more further biological activity assays to confirm that the antibody mutant(s) with enhanced binding affinity are indeed useful, e.g. for preclinical studies.

The antibody mutant(s) so selected may be subjected to further modifications, oftentimes depending on the intended use of the antibody. Such modifications may involve further alteration of the amino acid sequence, fusion to heterologous polypeptide(s) and/or covalent modifications such as those elaborated below. With respect to amino acid sequence alterations, exemplary modifications are elaborated above. For example, any cysteine residue not involved in maintaining the proper conformation of the antibody mutant also may be substituted, generally with serine, to improve the oxidative stability of the molecule and prevent aberrant cross linking. Conversely, cysteine bond(s) may be added to the antibody to improve its stability (particularly where the antibody is an antibody fragment such as an Fv fragment). Another type of amino acid mutant has an altered glycosylation pattern. This may be achieved by deleting one or more carbohydrate moieties found in the antibody, and/or adding one or more glycosylation sites that are not present in the antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

(xii) Recombinant Production of Antibodies

For recombinant production of an antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence (e.g. as described in U.S. Pat. No. 5,534,615, specifically incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serrafia, e.g, Serratia marcescans, and Shigeila, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X 1776 (ATCC 31,537), and E coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al, J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce the antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed cells, is removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human .γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH 3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

(xiii) Antibody Conjugates

In another aspect, the invention pertains to antibody conjugates or immunoconjugates comprising an antibody conjugated to a cytotoxic agent such as a chemotherapeutic agent, toxin (e.g. an enzymatically active toxin of bacterial, fungal, plant or animal origin, or fragments thereof), or a radioactive isotope (i.e., a radioconjugate).

Chemotherapeutic agents useful in the generation of such immunoconjugates have been described above. Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. A variety of radionuclides are available for the production of radioconjugated antibodies. Examples include ²¹²Bi, ¹³¹I, ¹³¹In, ⁹⁰Y and ¹⁸⁶Re.

Conjugates of the antibody and cytotoxic agent are made using a variety of bifunctional protein coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. Those of skill in the art will appreciate additional coupling agents suitable for the conjugates of the present invention.

In another embodiment, the antibody may be conjugated to a “receptor” (such streptavidin) for utilization in tissue pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

### CRTAM

V. CRTAM Modulator Polypeptides

In one aspect, a CRTAM modulator of the invention comprises a polypeptide. In one embodiment, the modulator polypeptide antagonizes CRTAM activity in an activated T cell. In one embodiment, the modulator polypeptide antagonizes Scrib activity in an activated T cell. In one embodiment, the polypeptide binds, preferably specifically, to CRTAM or an intracellular molecule that interacts with CRTAM such that biological activity associated with CRTAM-Scrib interaction is inhibited. In one embodiment, the modulator polypeptide agonizes CRTAM activity in an activated T cell. In one embodiment, the polypeptide binds, preferably specifically, to CRTAM such that biological activity associated with CRTAM is mimicked or enhanced. In one embodiment, the polypeptide binds, preferably specifically, to an extracellular molecule that interacts with CRTAM, such that biological activity associated with CRTAM-extracellular molecule is mimicked or enhanced. The polypeptides may be chemically synthesized using known peptide synthesis methodology or may be prepared and purified using recombinant technology. In one embodiment, a CRTAM modulator polypeptide is at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such polypeptides are capable of modulating CRTAM activity. These polypeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large oligopeptide libraries to identify member(s) of those libraries which are capable of specifically binding to a polypeptide target. Phage display is a technique by which variant polypeptides are displayed as fusion proteins to the coat protein on the surface of bacteriophage particles (Scott, J. K. and Smith, G. P. (1990) Science, 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren et al., Gene, 215: 439 (1998); Zhu et al., Cancer Research, 58(15): 3209-3214 (1998); Jiang et al., Infection & Immunity, 65(11): 4770-4777 (1997); Ren et al., Gene, 195(2):303-311 (1997); Ren, Protein Sci., 5: 1833 (1996); Efimov et al., Virus Genes, 10: 173 (1995)) and T7 phage display systems (Smith and Scott, Methods in Enzymology, 217: 228-257 (1993); U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties.

Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

Methods of generating, identifying, characterizing, modifying and producing modulator polypeptides are well established in the art, e.g., as described in US Pat. Appl. Pub. No. 2005/0042216 from paragraphs 606 through 608, 614 through 688.

VI. CRTAM Modulator Small Molecules

In one embodiment, CRTAM modulator small molecules are organic molecules other than oligopeptides or antibodies as defined herein that modulate CRTAM activity. CRTAM modulator small molecules may be identified and chemically synthesized using known methodology (see, e.g., PCT Publication Nos. WO00/00823 and WO00/39585). CRTAM modulator organic small molecules are usually less than about 2000 daltons in size, alternatively less than about 1500, 750, 500, 250 or 200 daltons in size, wherein such organic small molecules may be identified without undue experimentation using well known techniques. In this regard, it is noted that techniques for screening organic small molecule libraries for molecules that are capable of binding to a polypeptide target are well known in the art (see, e.g., PCT Publication Nos. WO00/00823 and WO00/39585). CRTAM modulator organic small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

VII. CRTAM Modulators Comprising Nucleic Acids

In one aspect, a CRTAM modulator of the invention comprises a nucleic acid molecule. For example, the nucleic acid molecule may comprise a sense/antisense oligonucleotide, an inhibitory/interfering RNA (e.g., a small inhibitory/interfering RNA (siRNA)), or an aptamer. Methods for screening for, identifying and making these nucleic acid modulators are known in the art.

For example, siRNAs have proven capable of modulating gene expression where traditional antagonists such as small molecules or antibodies have failed. (Shi Y., Trends in Genetics 19(1):9-12 (2003)). In vitro synthesized, double stranded RNAs that are fewer than 30 nucleotides in length (e.g., about 15 to 25, 17 to 20, 18 to 20, 19 to 20, or 21 to 23 nucleotides) can act as interfering RNAs (iRNAs) and can specifically inhibit gene expression (see, e.g., Fire A., Trends in Genetics (1999), 391; 806-810; U.S. patent application Ser. Nos. 09/821,832, 09/215,257; U.S. Pat. No. 6,506,559; PCT/US01/10188; European Appln. Ser. No. 00126325). These iRNAs are believed to act at least in part by mediating degradation of their target RNAs. However, since they are under 30 nuclotides in length, they do not trigger a cell antiviral defense mechanism. Such mechanisms include interferon production, and a general shutdown of host cell protein synthesis. Practically, siRNAs can be synthesized and then cloned into DNA vectors. Such vectors can be transfected and made to express the siRNA at high levels. The high level of siRNA expression is used to “knockdown” or significantly reduce the amount of protein produced in a cell, and thus it is useful in cellular settings where overexpression of a protein is believed to be linked to a pathological disorder.

Aptamers are nucleic acid molecules that are capable of binding to a target molecule, such as a CRTAM protein. The generation and therapeutic use of aptamers are well established in the art. See, e.g., U.S. Pat. No. 5,475,096, and the therapeutic efficacy of Macugen® (Eyetech, New York) for treating age-related macular degeneration.

Anti-sense technology is well established in the art. Further details regarding this technology are provided hereinbelow.

VIII. Formulations

Therapeutic formulations of the CRTAM modulators used in accordance with the invention are prepared for storage by mixing the CRTAM modulator having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG).

The formulations herein may also contain more than one active compound as necessary for the particular indication being treated, preferably those with complementary activities that do not adversely affect each other. For example, in addition to a CRTAM modulator, it may be desirable to include in the one formulation, an additional modulator, e.g., a second antibody which binds a different epitope on the CRTAM protein, or an antibody to some other target. Alternatively, or additionally, the composition may further comprise an immunosuppressive agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

The CRTAM modulator antibodies disclosed herein can be formulated in any suitable form for delivery to a target cell/tissue. For example, the antibodies may be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-288 (1982) via a disulfide interchange reaction. A chemotherapeutic agent is optionally contained within the liposome. See Gabizon et al., J. National Cancer Inst. 81(19):1484 (1989).

IX. Illustrative Uses of CRTAM Modulators of the Invention

CRTAM modulators of the invention have various non-therapeutic applications. The modulators can be useful for staging or detecting CRTAM-expressing diseases (e.g., in radioimaging). The antibodies, oligopeptides and organic small molecules are also useful for purification or immunoprecipitation of CRTAM from cells, for detection and quantitation of CRTAM in vitro, e.g., in an ELISA or a Western blot, to modulate cellular events in a population of cells.

Measurement of cells with certain surface markers in a sample may be performed by the techniques described herein. In some embodiments, measurements may be made using a software program executed by a suitable processor. Suitable software and processors are well known in the art and are commercially available. The program may be embodied in software stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

Following the measurement of the amount of cells having a particular cell surface marker and the determination that a subject is likely or not likely to have an autoimmune disease, the measurement results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. In some embodiments, the assays will be performed or the assay results analyzed in a country or jurisdiction which differs from the country or jurisdiction to which the results or diagnoses are communicated.

In one embodiment, a diagnosis, prediction and/or treatment recommendation based on the amount of cells measured in a test subject of having one or more of the surface markers herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, electronic forms of communication, such as email, or telephone. Communication may be facilitated by use of a computed, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions. CRTAM modulator antibodies of the invention can be in the different forms encompassed by the definition of “antibody” herein. Thus, the antibodies include full length or intact antibody, antibody fragments, native sequence antibody or amino acid variants, humanized, chimeric or fusion antibodies, and functional fragments thereof.

The invention provides a composition comprising a CRTAM modulator, and a carrier. In a further embodiment, a composition can comprise a CRTAM modulator in combination with other therapeutic agents such as immunosuppressive agents. The invention also provides formulations comprising a CRTAM modulator, and a carrier. In one embodiment, the formulation is a therapeutic formulation comprising a pharmaceutically acceptable carrier.

CRTAM modulators, for example antibodies of the invention, may be used in, for example, in vitro, ex vivo and in vivo therapeutic methods. Modulators of the invention can be used as an antagonist to partially or fully block the specific antigen activity in vitro, ex vivo and/or in vivo. Moreover, at least some of the modulators of the invention can neutralize antigen activity from other species. Accordingly, modulators of the invention can be used to inhibit a specific antigen activity, e.g., in a cell culture containing the antigen, in human subjects or in other mammalian subjects having the antigen with which a modulator of the invention cross-reacts (e.g. chimpanzee, baboon, marmoset, cynomolgus and rhesus, pig or mouse). In one embodiment, a modulator of the invention can be used for inhibiting antigen activities by contacting the modulator with the antigen such that antigen activity is inhibited. In one embodiment, the antigen is human CRTAM.

In one embodiment, a CRTAM modulator of the invention can be used in a method for inhibiting CRTAM in a subject suffering from a disorder in which CRTAM+ T cell activity is detrimental, comprising administering to the subject a modulator of the invention such that CRTAM activity in the subject is inhibited. In one embodiment, the subject is a human subject. Alternatively, the subject can be a mammal expressing CRTAM having an activity that is modulated by a modulator of the invention. Still further the subject can be a mammal into which CRTAM has been introduced (e.g., by administration of CRTAM or by expression of a CRTAM transgene). A modulator of the invention can be administered to a human subject for therapeutic purposes. Moreover, a modulator of the invention can be administered to a non-human mammal expressing CRTAM with which the modulator cross-reacts (e.g., a primate, pig or mouse) for veterinary purposes or as an animal model of human disease. Regarding the latter, such animal models may be useful for evaluating the therapeutic efficacy of modulators of the invention (e.g., testing of dosages and time courses of administration). Modulators of the invention can be used to treat, inhibit, delay progression of, prevent/delay recurrence of, ameliorate, or prevent diseases, disorders or conditions associated with abnormal expression and/or activity of CRTAM+ T cells, including but not limited to inflammatory, autoimmune and other immunologic disorders.

In certain embodiments, an immunoconjugate comprising an antibody conjugated with a cytotoxic agent is administered to the patient. In some embodiments, the immunoconjugate and/or antigen to which it is bound is/are internalized by the cell, resulting in increased therapeutic efficacy of the immunoconjugate in killing the target cell to which it binds. In one embodiment, the cytotoxic agent targets or interferes with nucleic acid in the target cell. Examples of such cytotoxic agents include any of the chemotherapeutic agents noted herein (such as a maytansinoid or a calicheamicin), a radioactive isotope, enzyme, or a small molecule toxin.

In one embodiment, a CRTAM modulator of the invention is used as an agonist to mimic or enhance biological activity associated with CRTAM in vitro, ex vivo and/or in vivo. Moreover, at least some of the modulators of the invention can mimic or enhance biological activity associated with CRTAM in other species. Accordingly, modulators of the invention can be used to mimic or enhance biological activity associated with CRTAM, e.g., in a cell culture, in human subjects, or in other mammalian subjects having CRTAM and with which a modulator of the invention cross-reacts (e.g. chimpanzee, baboon, marmoset, cynomolgus and rhesus, pig or mouse). In one embodiment, a modulator of the invention can be used for mimicking or enhancing biological activity associated with CRTAM by contacting the modulator with CRTAM such that biological activity associated with CRTAM is mimicked or enhanced. In one embodiment, the modulator comprises isolated Necl2 (Cadm1), or a fragment thereof, or a polypeptide fusion of Necl2 (Cadm1), or a polypeptide fusion of a fragment of Necl2 (Cadm1). In one embodiment, the modulator is a fusion of an extracellular domain of Necl2 with an Fc fragment of IgG.

In one embodiment, a CRTAM modulator of the invention can be used in a method for mimicking or enhancing biological activity associated with CRTAM in a subject suffering from a disorder in which CRTAM+ T cell activity is beneficial, comprising administering to the subject a modulator of the invention such that biological activity associated with CRTAM is mimicked or enhanced. In one embodiment, the subject is a human subject. Alternatively, the subject can be a mammal expressing CRTAM having an activity that is modulated by a modulator of the invention. Still further the subject can be a mammal into which CRTAM has been introduced (e.g., by administration of CRTAM or by expression of a CRTAM transgene). A modulator of the invention can be administered to a human subject for therapeutic purposes. Moreover, a modulator of the invention can be administered to a non-human mammal expressing CRTAM with which the modulator cross-reacts (e.g., a primate, pig or mouse) for veterinary purposes or as an animal model of human disease. Regarding the latter, such animal models may be useful for evaluating the therapeutic efficacy of modulators of the invention (e.g., testing of dosages and time courses of administration). Modulators of the invention can be used to treat, inhibit, delay progression of, prevent/delay recurrence of, ameliorate, or prevent diseases, disorders or conditions associated with expression and/or activity of CRTAM+ T cells, including but not limited to cancer, immune deficiency, and infection.

Modulators of the invention can be used either alone or in combination with other compositions in a therapy. For instance, a modulator of the invention, e.g., an antibody or polypeptide, may be co-administered with another antibody, and/or adjuvant/therapeutic agents (e.g., steroids). For instance, an antibody or polypeptide of the invention may be combined with an anti-inflammatory and/or antiseptic in a treatment scheme, e.g. in treating any of the diseases described herein, including inflammatory, autoimmune and other immunological disorders, as well as cancer, immune deficiency, and infection. Such combined therapies noted above include combined administration (where the two or more agents are included in the same or separate formulations), and separate administration, in which case, administration of the antibody or polypeptide of the invention can occur prior to, and/or following, administration of the adjunct therapy or therapies.

A modulator of the invention (and adjunct therapeutic agent) can be administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the modulator is suitably administered by pulse infusion, particularly with declining doses of the modulator. Dosing can be by any suitable route, e.g. by injections, such as intravenous or subcutaneous injections, depending in part on whether the administration is brief or chronic.

The modulator composition of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The modulator need not be, but is optionally formulated with one or more agents currently used to prevent or treat the disorder in question. The effective amount of such other agents depends on the amount of modulators of the invention present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a modulator of the invention (when used alone or in combination with other agents such as cytotoxic agents) will depend on the type of disease to be treated, the type of modulator, the severity and course of the disease, whether the modulator is administered for preventive or therapeutic purposes, previous therapy, the patient's clinical history and response to the modulator, and the discretion of the attending physician. The modulator is suitably administered to the patient at one time or over a series of treatments. Depending on the type and severity of the disease, about 1 μg/kg to 15 mg/kg (e.g. 0.1 mg/kg-10 mg/kg) of an antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of an antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

Aside from administration of a polypeptide modulator (e.g., polypeptide, antibody, etc.) to the patient, the invention contemplates administration of a modulator by gene therapy. Such administration of nucleic acid comprising/encoding the CRTAM modulator is encompassed by the expression “administering a therapeutically effective amount of a CRTAM modulator”. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the CRTAM modulator is required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

In one embodiment, in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of the currently known gene marking and gene therapy protocols see Anderson et al., Science 256:808-813 (1992). See also WO 93/25673 and the references cited therein.

X. Articles of Manufacture and Kits

Another embodiment of the invention is an article of manufacture containing materials useful for the treatment of a disorder using a CRTAM modulator. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is a CRTAM modulator of the invention. The label or package insert indicates that the composition is used for treating a particular disorder. The label or package insert will further comprise instructions for administering the composition to the patient. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes. Kits can be provided which contain CRTAM modulators of the invention for detection and quantitation of CRTAM in vitro, e.g., in an ELISA or a Western blot. As with the article of manufacture, the kit comprises a container and a label or package insert on or associated with the container. The container holds a composition comprising at least one CRTAM modulator of the invention. Additional containers may be included that contain, e.g., diluents and buffers, control antibodies. The label or package insert may provide a description of the composition as well as instructions for the intended in vitro or detection use.

XI. CRTAM Modulators Comprising Polypeptides or Nucleic Acids—Specific Forms and Applications

In one embodiment, nucleic acids of the invention include antisense or sense oligonucleotides/polynucleotides comprising a singe-stranded nucleic acid sequence (either RNA or DNA) capable of binding to endogenous CRTAM nucleic acids or that encode CRTAM polypeptide. Antisense or sense oligonucleotides, according to the present invention, comprise at least a fragment of the coding region of CRTAM DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. Such methods are encompassed by the invention. The antisense oligonucleotides thus may be used to block expression of a CRTAM protein in activated T cells. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Preferred intragenic sites for antisense binding include the region incorporating the translation initiation/start codon (5′-AUG/5′-ATG) or termination/stop codon (5′-UAA, 5′-UAG and 5-UGA/5′-TAA, 5′-TAG and 5′-TGA) of the open reading frame (ORF) of the gene. These regions refer to a portion of the mRNA or gene that encompasses from about 25 to about 50 contiguous nucleotides in either direction (i.e., 5′ or 3′) from a translation initiation or termination codon. Other exemplary regions for antisense binding include: introns; exons; intron-exon junctions; the open reading frame (ORF) or “coding region,” which is the region between the translation initiation codon and the translation termination codon; the 5′ cap of an mRNA which comprises an N7-methylated guanosine residue joined to the 5′-most residue of the mRNA via a 5′-5′ triphosphate linkage and includes 5′ cap structure itself as well as the first 50 nucleotides adjacent to the cap; the 5′ untranslated region (5′UTR), the portion of an mRNA in the 5′ direction from the translation initiation codon, and thus including nucleotides between the 5′ cap site and the translation initiation codon of an mRNA or corresponding nucleotides on the gene; and the 3′ untranslated region (3′UTR), the portion of an mRNA in the 3′ direction from the translation termination codon, and thus including nucleotides between the translation termination codon and 3′ end of an mRNA or corresponding nucleotides on the gene.

Specific examples of antisense compounds useful for inhibiting expression of CRTAM polypeptide include oligonucleotides containing modified backbones or non-natural internucleoside linkages. Oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides. Exemplary modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Exemplary oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included. Representative United States patents that teach the preparation of phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Examples of modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts. Representative United States patents that teach the preparation of such oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

In other examples of antisense oligonucleotides, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Examples of antisense oligonucleotides incorporate phosphorothioate backbones and/or heteroatom backbones, and in particular —CH₂—NH—O—CH₂—, —CH₂—N(CH₃)—O—CH₂— [known as a methylene(methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] described in the above referenced U.S. Pat. No. 5,489,677, and the amide backbones of the above referenced U.S. Pat. No. 5,602,240. Additional examples are antisense oligonucleotides having morpholino backbone structures of the above-referenced U.S. Pat. No. 5,034,506.

Modified oligonucleotides may also contain one or more substituted sugar moieties. Exemplary oligonucleotides comprise one of the following at the 2′ position: OH; F; O-alkyl, S-alkyl, or N-alkyl; O-alkenyl, S-alkeynyl, or N-alkenyl; O-alkynyl, S-alkynyl or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other exemplary antisense oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One possible modification includes 2′-methoxyethoxy (2′-O—CH₂CH₂OCH₃, also known as 2′-O-(2-methoxyethyl) or 2′-MOE) (Martin et al., Hely. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂).

A further modification includes Locked Nucleic Acids (LNAs) in which the 2′-hydroxyl group is linked to the 3′ or 4′ carbon atom of the sugar ring thereby forming a bicyclic sugar moiety. The linkage can be a methelyne (—CH₂—)ₙ group bridging the 2′ oxygen atom and the 4′ carbon atom wherein n is 1 or 2. LNAs and preparation thereof are described in WO 98/39352 and WO 99/14226.

Other modifications include 2′-methoxy (2′-O—CH₃), 2′-aminopropoxy (2′-OCH₂CH₂CH₂ NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. One 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃ or —CH₂—C═CH) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine(1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine(2H-pyrimido[4,5-b]indol-2-one), pyridoindole cytidine(H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi et al, Antisense Research and Applications, CRC Press, Boca Raton, 1993, pp. 276-278) and are exemplary base substitutions, e.g., when combined with 2′-O-methoxyethyl sugar modifications. Representative United States patents that teach the preparation of modified nucleobases include, but are not limited to: U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941 and 5,750,692, each of which is herein incorporated by reference.

Another modification of antisense oligonucleotides comprises chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, cation lipids, phospholipids, cationic phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) and U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Chimeric antisense compounds of the invention may be formed as composite structures of two or more oligonucleotides, modified oligonucleotides, oligonucleosides and/or oligonucleotide mimetics as described above. Exemplary chimeric antisense oligonucleotides incorporate at least one 2′ modified sugar (preferably 2′-O—(CH₂)₂—O—CH₃) at the 3′ terminal to confer nuclease resistance and a region with at least 4 contiguous 2′-H sugars to confer RNase H activity. Such compounds have also been referred to in the art as hybrids or gapmers. Exemplary gapmers have a region of 2′ modified sugars (preferably 2′-O—(CH₂)₂—O—CH₃) at the 3′-terminal and at the 5′ terminal separated by at least one region having at least 4 contiguous 2′-H sugars and may incorporate phosphorothioate backbone linkages. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In an exemplary procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. In general, conjugation of the ligand binding molecule preferably does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

Nucleic acid encoding a CRTAM modulator polypeptide may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

CRTAM modulator polypeptides and nucleic acid molecules of the invention may be used diagnostically for tissue typing, wherein CRTAM polypeptides may be differentially expressed in one tissue as compared to another, preferably in a diseased tissue as compared to a normal tissue of the same tissue type.

This invention encompasses methods of screening compounds to identify those that modulate CRTAM. Screening assays for antagonist drug candidates are designed to identify compounds that bind or complex with the CRTAM polypeptide, or otherwise interfere with the interaction of the CRTAM polypeptides with other cellular proteins, including e.g., inhibiting the expression of CRTAM polypeptide from cells. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a CRTAM polypeptide under conditions and for a time sufficient to allow these two components to interact.

In binding assays, the interaction is binding and the complex formed can be isolated or detected in the reaction mixture. In a particular embodiment, the CRTAM polypeptide or the drug candidate is immobilized on a solid phase, e.g., on a microtiter plate, by covalent or non-covalent attachments. Non-covalent attachment generally is accomplished by coating the solid surface with a solution of the CRTAM polypeptide and drying. Alternatively, an immobilized antibody, e.g., a monoclonal antibody, specific for the CRTAM polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a CRTAM polypeptide, its interaction with CRTAM can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of CRTAM and other intra- or extracellular components can be tested as follows: usually a reaction mixture is prepared containing CRTAM and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the CRTAM polypeptide may be added to a cell along with the compound to be screened for a particular activity and the ability of the compound to inhibit the activity of interest in the presence of the CRTAM polypeptide indicates that the compound is an antagonist to the CRTAM polypeptide. The CRTAM polypeptide can be labeled, such as by radioactivity, such that the number of CRTAM polypeptide molecules bound to a receptor molecule can be used to determine the effectiveness of the potential antagonist.

A potential CRTAM antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence which encodes the mature CRTAM protein can be used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the CRTAM polypeptide. The antisense RNA oligonucleotide hybridizes to the mRNA in vivo and blocks translation of the mRNA molecule into the CRTAM polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the CRTAM polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the protein interaction site, or other relevant binding site of the CRTAM polypeptide, thereby blocking the normal biological activity of the CRTAM polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

In one embodiment, internalizing antibodies are preferred. Antibodies can possess certain characteristics, or modified to possess such characteristics, that enhance delivery of antibodies into cells. Techniques for achieving this are known in the art. In yet another embodiment, an antibody can be expressed in a target cell by introducing a nucleic acid capable of expressing the antibody into a targeted cell. See, e.g., U.S. Pat. Nos. 6,703,019; 6,329,173; and PCT Pub. No. 2003/077945. Lipofections or liposomes can also be used to deliver the antibody into cells. Where antibody fragments are used, the smallest inhibitory fragment that specifically binds to the binding domain of the target protein is generally advantageous. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

Entry of modulator polypeptides into target cells can be enhanced by methods known in the art. For example, certain sequences, such as those derived from HIV Tat or the Antennapedia homeodomain protein are able to direct efficient uptake of heterologous proteins across cell membranes. See, e.g., Chen et al., Proc. Natl. Acad. Sci. USA (1999), 96:4325-4329.

The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, or other immunosuppressive agent. Such molecules are suitably present in combination in amounts that are effective for the purpose intended.

### Deposit of Materials

The following hybridoma cell lines have been deposited with the American Type Culture Collection, 10801 University Boulevard, Manassas, Va. 20110-2209, USA (ATCC):

| Antibody Designation | ATCC No. | Deposit Date |
|---|---|---|
| 34G4.6.2.1.1.3 | PTA-8460 | May 24, 2007 |
| 32D6.4.1.1.1 | PTA-8461 | May 24, 2007 |
| 6E2.27.8.2.1 | PTA-8462 | May 24, 2007 |
| 17B2.7.12.9.2.2 | PTA-8463 | May 24, 2007 |
| 20F4.1.1.1.2.1 | PTA-8464 | May 24, 2007 |

The following are examples of the methods and compositions of the invention. It is understood that various other embodiments may be practiced, given the general description provided above. The examples are offered for illustrative purposes only, and are not intended to limit the scope of the present invention in any way.

### Examples

### Experimental Procedures

### Mice

C57BL/6 mice were purchased from The Jackson Laboratory (Bar Harbor, Me.). The Crtam-knockout mice, were produced in collaboration between Genentech and Lexicon Genetics (The Woodlands, Tex.) to analyze the function of about 500 secreted and transmembrane proteins. The specific targeting strategy is described below.

### Immunofluorescence Microscopy

For studies on T cell polarity, unstimulated naïve CD4⁺ T cell or 14 h-activated T cells were fixed with 4% paraformaldehyde, stained with anti-Crtam hamster polysera, anti-CD44 mAb (BD Pharmingen) or anti-CD3 mAb (BD Pharmingen), permeabilized with 0.2% Triton X-100, and stained with anti-Talin (Sigma), anti-PKCθ (Cell Signaling), anti-Scrib (H-300), anti-Cdc42 (B-8), or anti-PKCζ (H-1) antibodies from Santa Cruz Biotechnology. Slides were analyzed by deconvolution microscopy (Delta Vision). The 3D models of staining were reconstituted using Imaris 5.5.

To assess interactions between T and dendritic (DC) cells, DCs were positively purified by CD11c MicroBeads (Miltenyi Biotec) and labeled with 0.5 μM Cell Tracker Orange CMRA (Molecular Probes). After incubation with 5 μM OVA₃₂₃₋₃₃₉ for 1 h, DCs were incubated with naïve CD4⁺ T cells from Crtam^{+/+} OT-II TCR^{tg} and Crtam^{-/-} OT-II TCR^{tg} mice at 37° C. for 30 min on Poly-D-Lysine-coated coverslips (BD BioCoat™). Cells were fixed with 4% paraformaldehyde in PBS, permeabilized with 0.2% Triton X-100 and stained with phalloidin (Molecular Probes) for F-actin. DAPI-containing mounting medium (Vector Laboratories) was used to visualize DNA.

### Retroviral Reconstitution

Retroviral constructs for wild type Crtam, ΔICD (amino acids 1-316), ΔECD (amino acids 251-393), or ΔESIV mutants also encoded a downstream internal ribosomal entry site promoter driven eGFP (Pear et al., 1993). Purified Crtam^{-/-} OT-II TCR^{tg} CD4⁺ T cells were activated and spin-transfected (90 min at 1800 rpm) with supernatants from retroviral DNA-transfected Phoenix cells. eGFP⁺ cells were sorted based on comparable levels of Crtam expression. Sorted cells were re-stimulated two days later. T cell polarity was examined 14 h after re-stimulation while thymidine incorporation and cytokine production was measured at 48 h.

### Flow Cytometry and Proliferation Assays

Single cell suspensions were stained with monoclonal antibodies (mAb) against CD3, CD4, CD8, B220, CD44, CD25, CD62L, CD45RB or CD69 (BD Pharmingen), hamster polysera against Crtam, or Crtam mAb (17B2, Genentech). Microbead-enriched naïve CD4⁺ or CD8⁺ T cells (2×10⁴) from spleens of Crtam^{+/+} and Crtam^{-/-} mice were cultured in flat-bottomed MaxiSorp surface microplate (Nunc) coated with 10 μg/ml anti-CDR mAb and 2 μg/ml anti-CD28 mAb or irradiated 5 μM OVA₃₂₃₋₃₃₉ pulsed antigen presenting cells. After 42 h, [³H]thymidine (1 μCi/well) was added and the plates were harvested 8 h later. For Carboxyfluorescein diacetate-succinimidyl ester (CFSE) labeling, the cells were labeled with 5 μM CFSE by CellTrace™ CFSE Cell Proliferation Kit (Molecular Probes, Portland, Oreg.) and proliferating cells harvested 3 days later for FACScan analysis.

### Western Blotting

To assess the interaction of Crtam and PDZ-containing proteins, microbead purified CD4⁺ T cells were stimulated with plate-bound anti-CD2ε (10 μg/ml) and anti-CD28 (2 μg/ml) mAbs, whole cell extracts were immunoprecipitated using anti-Scrib antibody (K-21, Santa Cruz) and immune complexes analyzed by immunoblotting with anti-Crtam mAb (6E2, Genentech) or anti-Scrib Ab (H-300, Santa Cruz). The control experiment was performed using anti-Dlg1 Ab (H-60, Santa Cruz) for immunoprecipitation. In the pull down experiment, Flag-tagged Crtam were expressed in 293 cells and cell lysates were incubated with various GST-PDZ proteins as indicated at 4° C. overnight (Zhang et al., 2006). Crtam-Flag was immunoprecipitated using anti-Flag M2-agarose beads (Sigma) and immune complexes were analyzed by immunoblotting with anti-GST (GE Healthcare Bio-Sciences) or anti-Crtam (6E2) mAbs.

### Crtam(ΔICD):Scrib Chimeric Receptor Construction

To generate the Crtam(ΔICD):Scrib chimera, the full-length MmScrib cDNA was cloned from the mRNA of naïve mouse T cells, and fused to the C-terminal of Crtam(ΔICD, aa 1-316) using a BamHI site. The Crtam(ΔICD):Scrib chimera was further sub-cloned into pIRES2-EGFP vector. 4 μg of plasmid DNA was used in electroporation with 5×10⁶ T cells using Mouse T cell Nucleofector kit (Amaxa) and Amaxa Nucleofector (program X-01).

### Crtam(AECD) FLAG Epitope Tagged Mutant Receptor Construction

To generate the Crtam(ΔECD) Flag epitope tagged mutant, the endogenous Crtam signal sequence was fused to the N-terminus of the Flag peptide, and the tramsmembrane and intracellular domains of Crtam (aa 251-393) were fused to the C-terminus of the Flag peptide. This construct was expressed in a retroviral vector encoding GFP.

### Cadm1(ECD)-Fc Construction

To generate Cadm1(ECD)-Fc, the ECD of Cadm1 (aa 1-374) was fused to the Fc-domain of human IgG1. This construct was transiently expressed from a plasmid vector in CHO cells.

Scrib siRNA

The siRNAs used in this study are listed as follow: MmScrib1, target sequence AGGGAAGACGGTGAAAGTGAA (SEQ ID NO: 9) (QIAGEN, cat. #S101411851); MmScrib2, target sequence CCGGATGGCTTCACACAGCTA (SEQ ID NO: 10) (QIAGEN, cat. #S101411858); MmScrib3, target sequence CGGAACGATATTCCTGAGATA (SEQ ID NO:11) (QIAGEN, cat. #S101411865); MmScrib4, target sequence CTGGAGGGACTTACCCACCTA (SEQ ID NO: 12) (QIAGEN, cat. #S101411872); Ctrl-AllStars1 (QIAGEN, cat. #S103650318). The concentration of siRNA used for transfection was 0.25 μg each for a total 1 μg of Scrib siRNA mixture and 1 μg of control siRNA. Conditions for electroporation are the same as those described above using the Mouse t cell Nucleofector kit (Amaxa).

### Generation of Crtam-Deficient Mice

The Crtam-knockout mice were produced in a collaboration between Genentech and Lexicon Genetics (The Woodlands, Tex.) to analyze the function of about 500 secreted and transmembrane proteins. Crtam-deficient mice were generated by Lexicon Genetics Inc (The Woodlands, Tex.) by using strategies described in FIG. 9A. The targeting vector, pKOS-11, replaced exon1 of the mouse Crtam locus with a neomycin resistance cassette by homologous recombination. The targeting construct was electroporated into 129 strain embryonic stem (ES) cells and targeted clones were identified. Targeted ES clones were microinjected into C57BL/6 blastocysts, and the resulting male chimeras were crossed with female C57BL/6 mice to generate F1 Crtam heterozygous germline mice. Heterozygotes are intercrossed to produce F2 wild type, heterozygote and homozygote mice.

Mice used in these studies were genotyped by tail DNA via PCR using primers as follow: 5′-GACACAGGCAAGGTCACAGA-3′ (SEQ ID NO:13) with 5′-AGAGTAACTGCCCTTGGACGTG-3′ (SEQ ID NO: 14) or 5′-GCAGCGCATCGCCTTCTATC-3′ (SEQ ID NO: 15) generating wild type 328-bp fragment and knock-out 191-bp fragment. Reactions contained mouse DNA (200 ng) using RedMix (Sigma). Reactions were amplified as follows: denaturation 94° C. for 4 min, 30 cycles of 95° C. for 1 min, 60° C. for 30 s and 72° C. for 1 min and a final extension of 72° C. for 10 min. The probes used for genomic southern blotting were prepared by C57BL-6 genomic DNA via PCR using primers as follow: 5′-GTCTGCCAAGTTCCTTGTACAC-3′ (SEQ ID NO: 16) with 5′-ACTGGGCTCTCACTTCTTAATC-3′ (SEQ ID NO: 17) generating 392-bp 5′ probe and 5′-TTGGTTTTGGGAGCTTAATTCT-3′ (SEQ ID NO: 18) with 5′-GTATAGAGCTCAGGGAATTGAA-3′ (SEQ ID NO: 19) generating 367-bp 3′ probe. The probes were labeled using PCR DIG Probe Synthesis Kit (Roche Molecular Biochemicals). Electroporated DNA fragments were transferred to positive charged nylon membranes, and DIG Wash and Block Buffer Set (Roche Molecular Biochemicals) and anti-Digoxigenin-AP Fab fragments were used to reveal the genomic fragments.

### Reverse Transcription-Polymerase Chain Reaction

RNA from spleens of Crtam^{+/+} and Crtam^{-/-} mice were extracted using TRIZOL Regent (Invitrogen Life Technologies) and reverse transcribed with oligo d(T)₁₆ by using SuperScript First-Strand Synthesis System for RT-PCR (Invitrogen) according to the manufacturer's protocol. The cDNA served as template for the amplification of Crtam using primers 5′-GGCAGAATGGAGAGAAATCG-3′ (SEQ ID NO: 20) with 5′-CCAGTGTGAGCAGCAGGATA-3′ (SEQ ID NO:21) generating 498-bp fragment. Conventional PCR reactions were performed with an initial denaturation step of 95° C. for 2 min, followed by 30 cycles of 94° C. for 1 min, 60° C. for 30 s, 72° C. for 1 min and a final elongation step for 72° C. for 7 min.

### ELISA

For IFNγ, IL4, IL22, and IL17 production, supernatants from the activated T cells were harvested at 48 h after stimulation. Concentrations of IFNγ and IL17 were determined with Quantikine Mouse Immunoassay (R&D Systems), IL4 determined with BD OptEIA Set (BD Bioscience) and IL22 determined as described previously (Zheng et al., 2007). Analysis was read out using a microplate reader set at 450 nm and wavelength correction set to 540 nm following manufacturer's protocol.

In Vitro Differentiation of Naïve CD4⁺ T Cells

Mouse splenic CD4⁻ T cells from C57BL/6 Crtam^{+/+} and Crtam^{-/-} mice were negatively enriched using a CD4⁺ T cell isolation kit (Miltenyi Biotec) and naïve CD4⁺ T cells (CD4⁻CD62L⁺) were positively selected using CD62L Microbeads. T cells were cultivated as described previously (Batten et al., 2006). Purified naïve CD4⁺ T cells (1×10⁶ cells/ml) were activated with plate-bound anti-CD3 (10 μg/ml) and anti-CD28 (2 μg/ml) under the T_{H}1 condition [recombinant mouse IL12 (3.5 ng/ml, R&D Systems) and rat anti-mouse IL4 mAb (5 μg/ml, BD Pharmingen)], T_{H}2 condition [recombinant mouse IL4 (3.5 ng/ml, R&D Systems), hamster anti-mouse IFNγmAb (5 μg/ml, BD Pharmingen) and rat anti-mouse IL12 mAb (5 μg/ml, BD Pharmingen)], or T_{H}17 condition [anti-IFNγ mAb, anti-IL4 mAb and hsTGFβ1 (1 ng/ml, R&D Systems) with recombinant mouse IL6 (10 ng/ml, R&D Systems) or recombinant mouse IL23 (10 ng/ml, eBioscience)]. C57BL/6 CD4⁺ T cells, activated for 14 hours, were stained for Crtam expression and Crtam⁺ or Crtam⁻ T cells purified by FACS sorting. Highly purified Crtam⁺ and Crtam⁻ T cells were then cultivated under the initial differentiating conditions for 5 more days. Differentiated T cells were washed, counted and re-stimulated with plate-bound anti-CD3 (10 μg/ml) and anti-CD28 (2 μg/ml) for the analysis of cytokine production.

### TaqMan Real-Time Quantitative Reactions

Naïve CD4⁺ T cells from C57BL/6 mice were activated for 14 hours. Crtam⁺ and Crtam⁻ T cells were purified by FACS sorting. RNA was extracted using TRIZOL Regent (Invitrogen life technologies) and reverse transcribed with oligo d(T)₁₆ using SuperScript First-Strand Synthesis System for RT-PCR (Invitrogen) according to the manufacturer's protocol. The cDNA served as template for the amplification of genes of interest. Real-time quantitative reactions were performed with 50 pmol of each primer and 10 pmol of the FAM- and TAMRA-labeled probe in a 20 μl total reaction volume by using the TaqMan PCR Core Reagents Kit (Applied Biosystems, Foster City, Calif.). Samples were subjected to 40 cycles of amplification using an ABI Prism 7700 Sequence Detection System instrument (PE Applied Biosystems) according to the manufacturer's protocol for TaqMan assay PCR cycling conditions. Gene expression was presented as 2^{-Δct} and the values are expressed as arbitrary units (relative to housekeeping gene, RPL-19 levels). The primers used for TaqMan reaction were listed as follow: IFNγ: Forward, 5′-TCTACCTCAGACTCTTTGAAGTCTTG-3′ (SEQ ID NO: 22), Reverse, 5′-GGTGTGATTCAATGACGCTTAT-3′ (SEQ ID NO: 23), and Probe, 5′-AAGACAATCAGGCCATCAGCAACAAC-3′ (SEQ ID NO:24); IL22: Forward, 5′-GACAGGTTCCAGCCCTACA-3′ (SEQ ID NO: 25), Reverse, 5′-GAGCTGATTGCTGAGTTTGG-3′ (SEQ ID NO: 26), and Probe, 5′-CAGGAAAGGCACCACCTCCTGC-3′ (SEQ ID NO: 27); IL2: Forward, 5′-AAAGGGCTCTGACAACACATT-3′ (SEQ ID NO: 28), Reverse, 5′-TCAGAAAGTCCACCACAGTTG-3′ (SEQ ID NO: 29), and Probe, 5′-TGACTCATCATCGAATTGGCACTCA-3′ (SEQ ID NO: 30).

### FACS-Based Conjugation Assay

Purified DCs, used as the APCs, were labeled with Cell Tracker Orange CMRA (Molecular Probes) and pulsed with various concentration of OVA₃₂₃₋₃₃₉ as indicated. CD4⁻ T cells from Crtam^{+/+} OT-II TCR^{tg} and Crtam^{-/-} OT-II TCR^{tg} mice were labeled with Cell Tracker Green 5-chloromethylfluorescein diacetate (CMFDA, Molecular Probes, Eugene, Oreg.). Both DCs and T cells were resuspended in cold DMEM at a concentration of 5×10⁶ cells/ml. For conjugation assays, equal numbers of dendritic and T cells were added together, pelleted briefly at room temperature and incubated at 37° C. for 10 min. Cells were fixed in 0.5% paraformaldehyde. The relative proportion of red, green, and red/green events was determined by flow cytometry.

### Results

### Crtam is Transiently Expressed on a Subset of Activated CD4⁺ T Cells

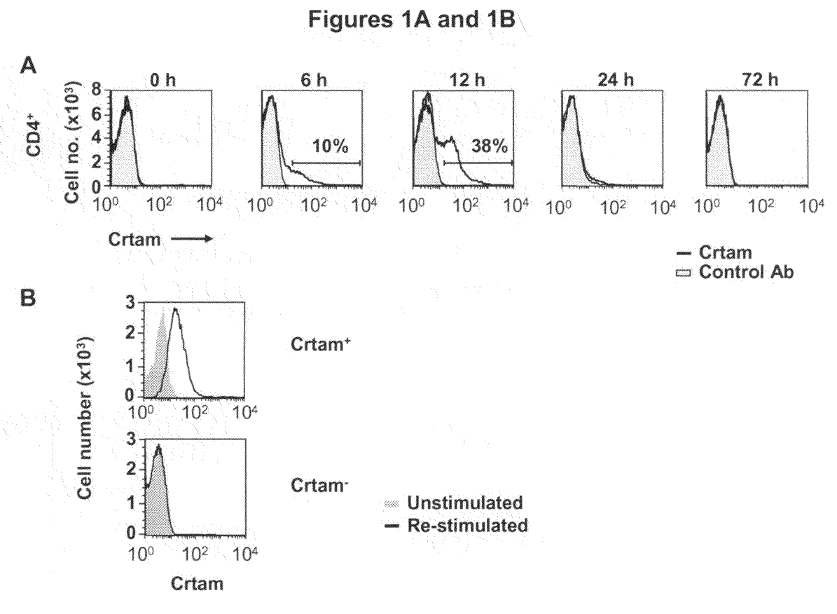

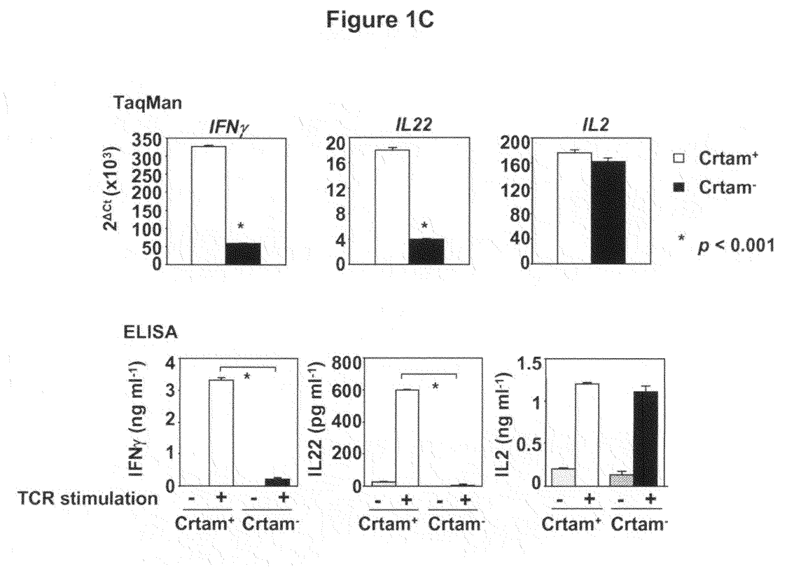

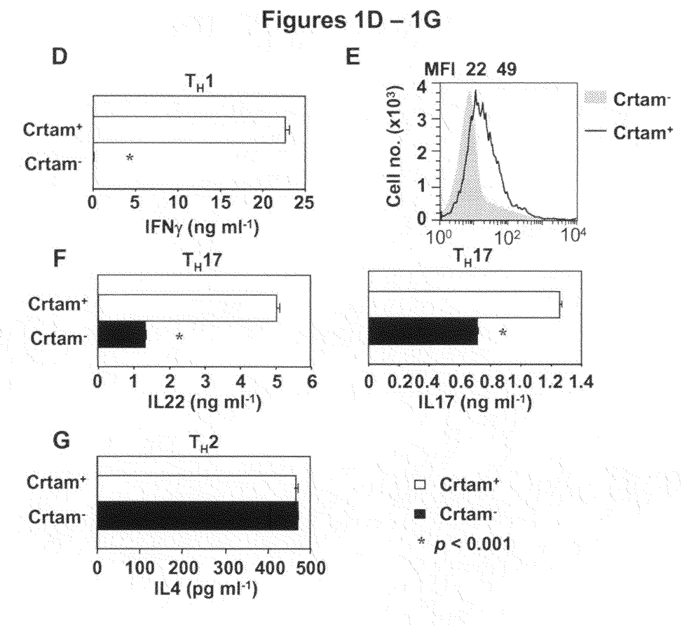

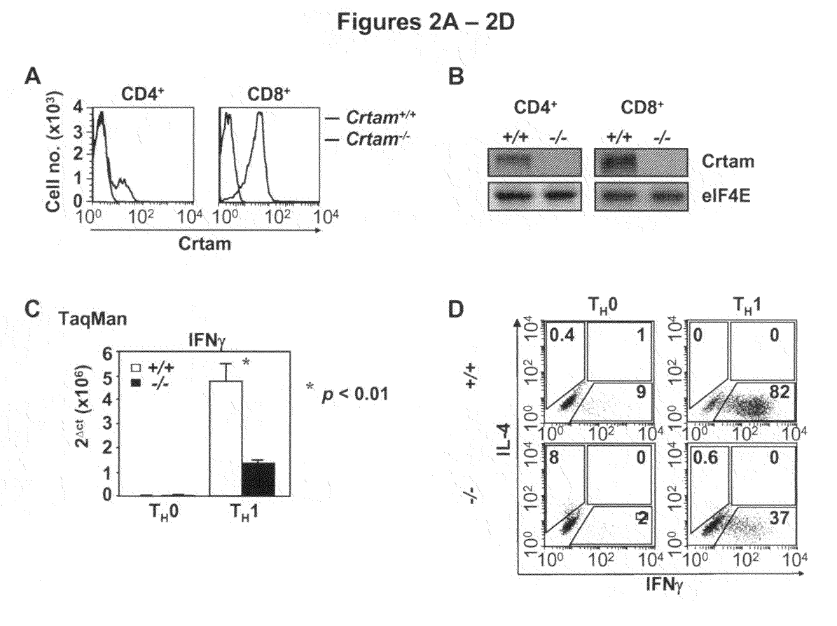

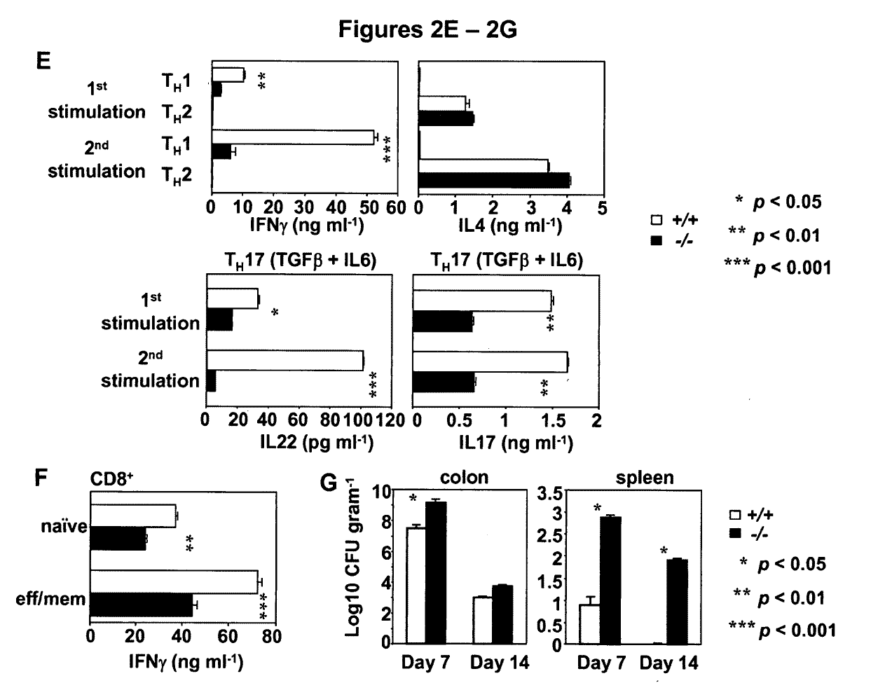

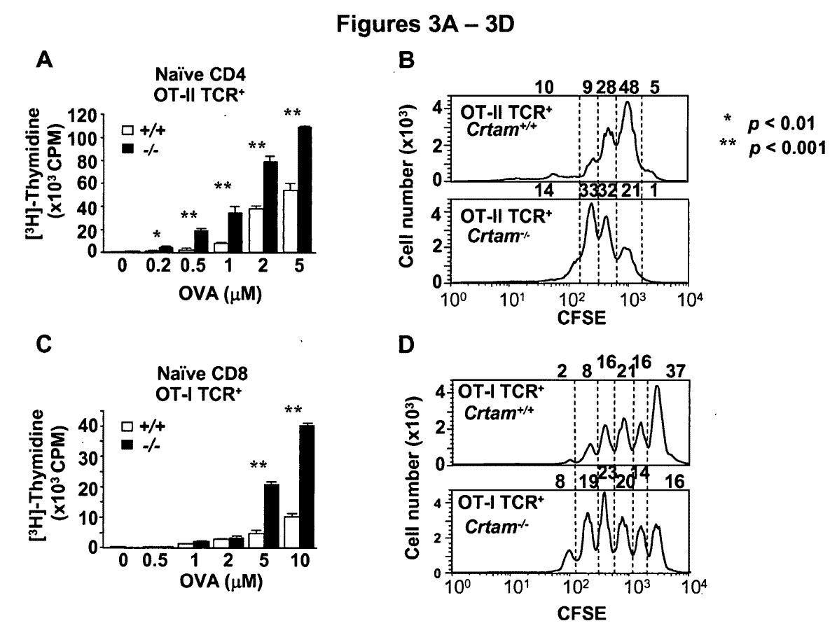

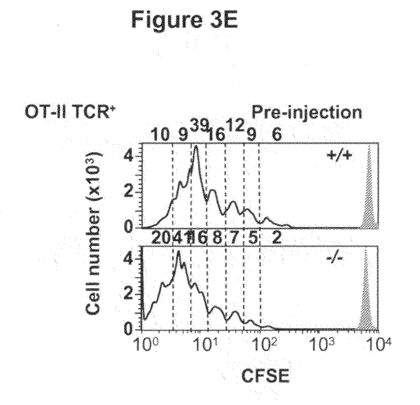

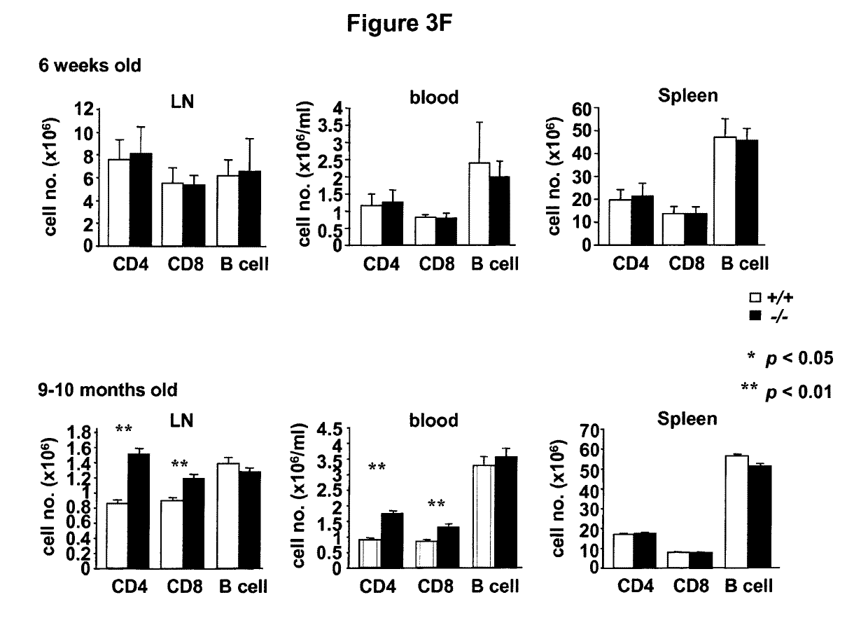

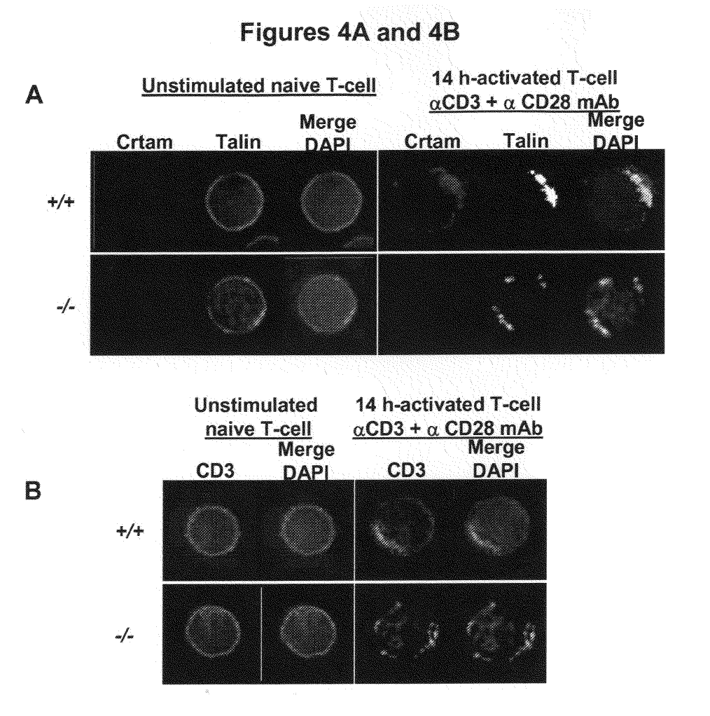

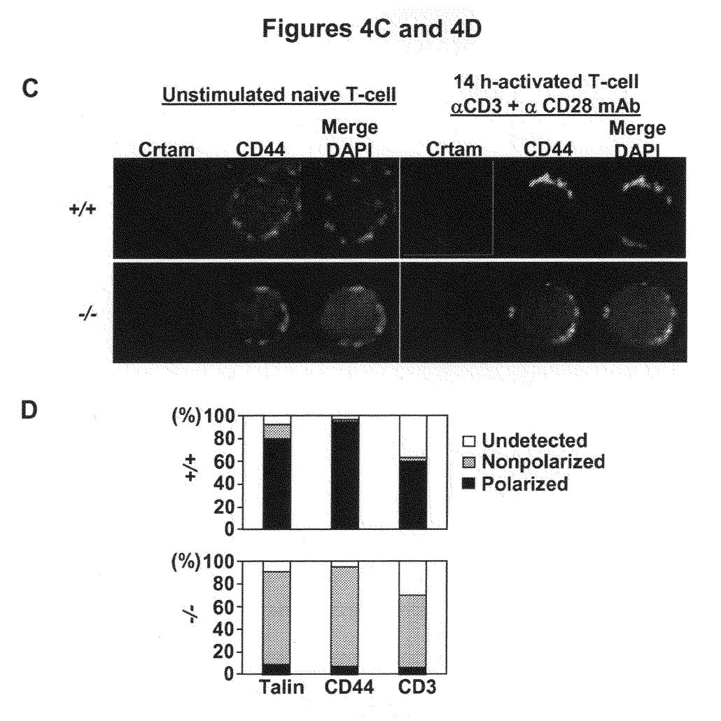

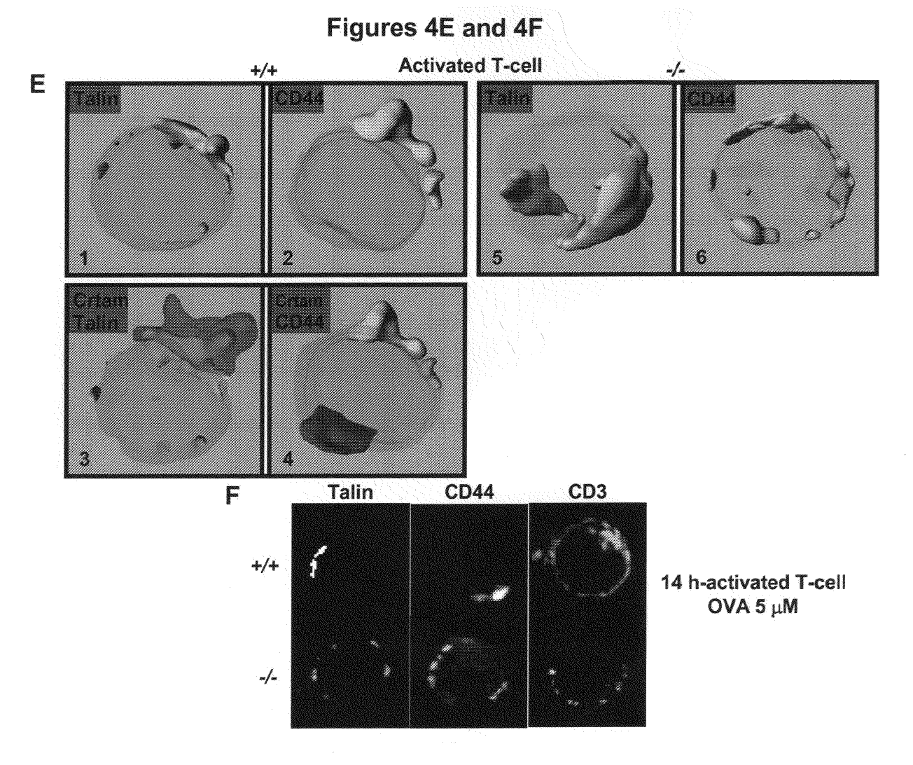

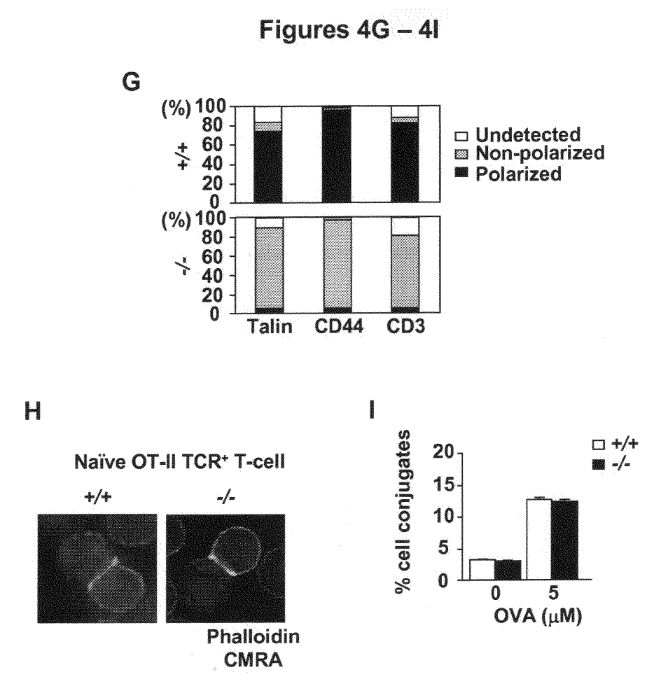

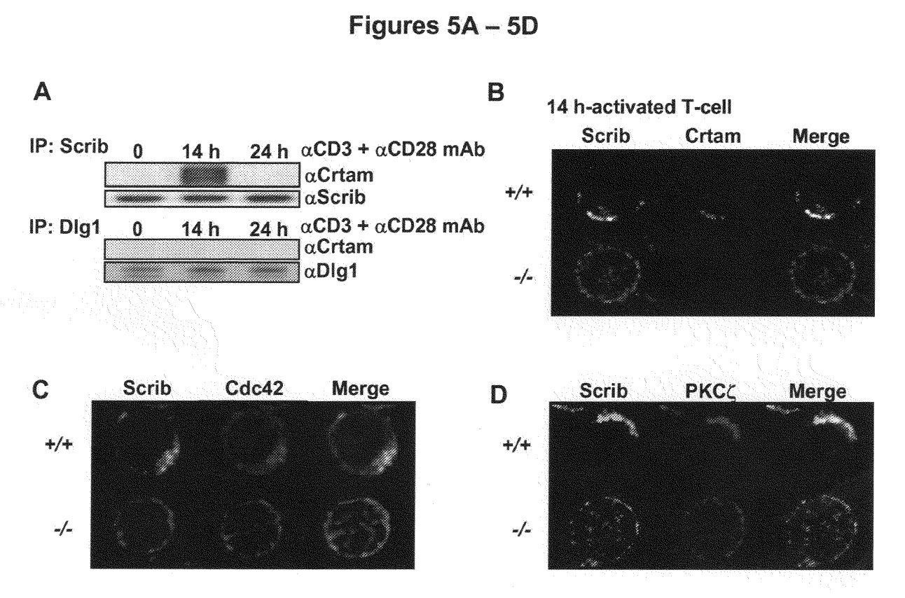

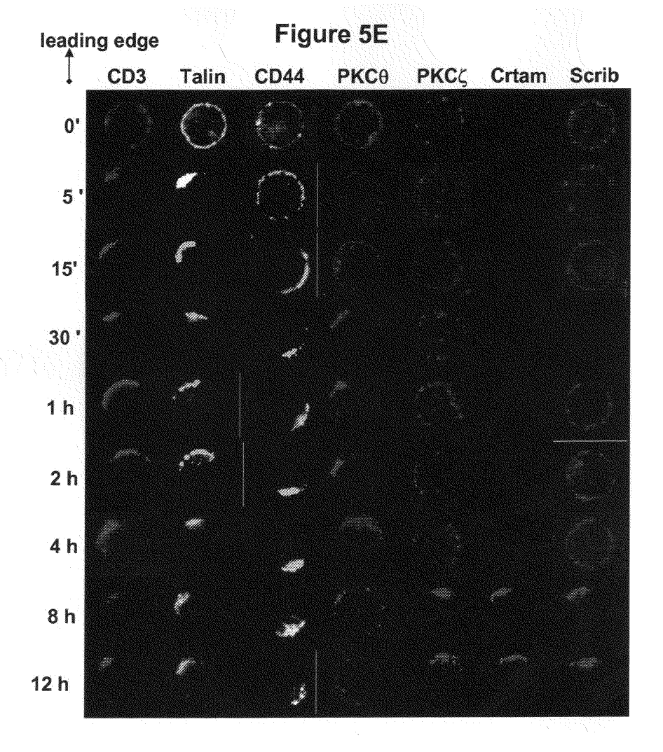

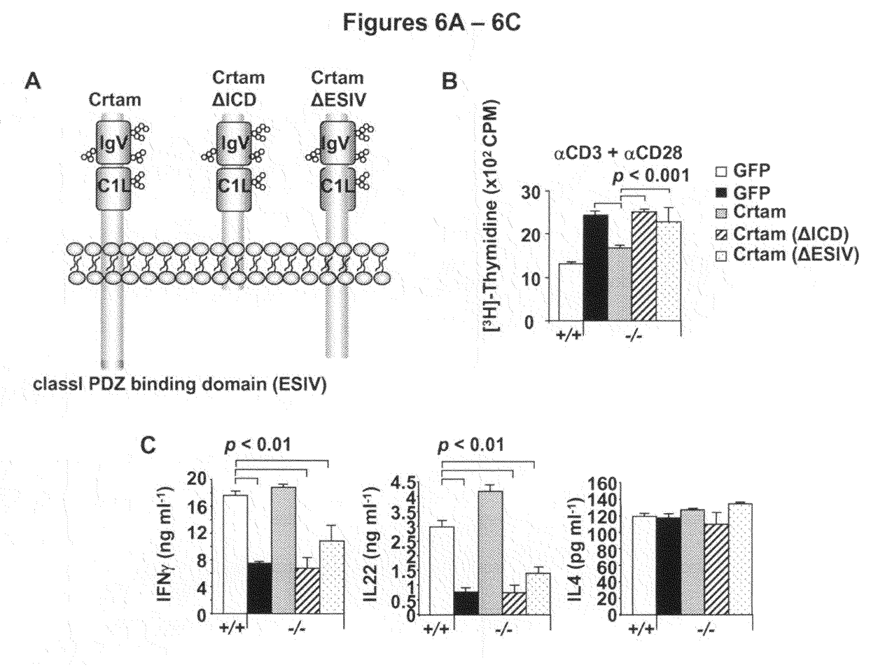

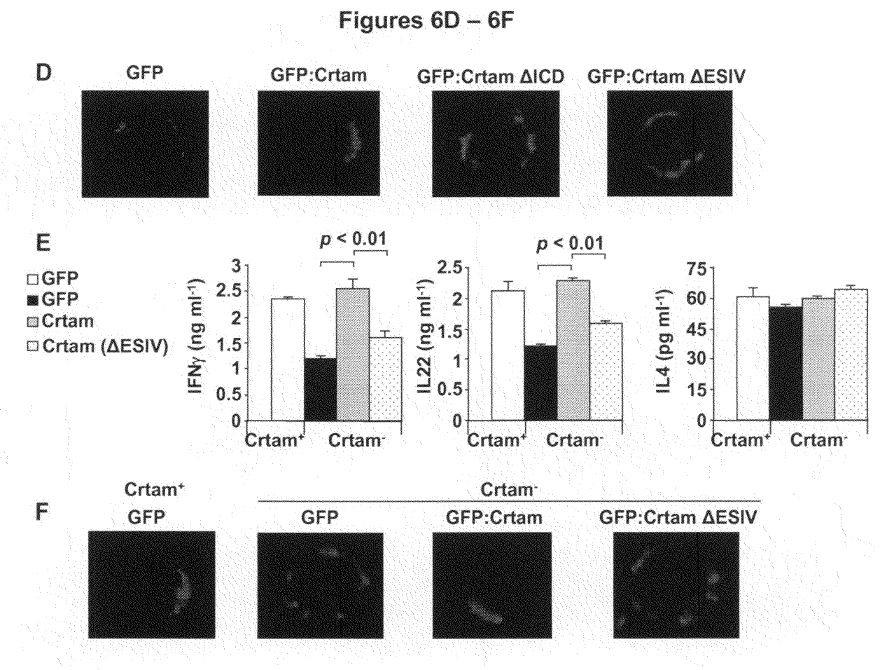

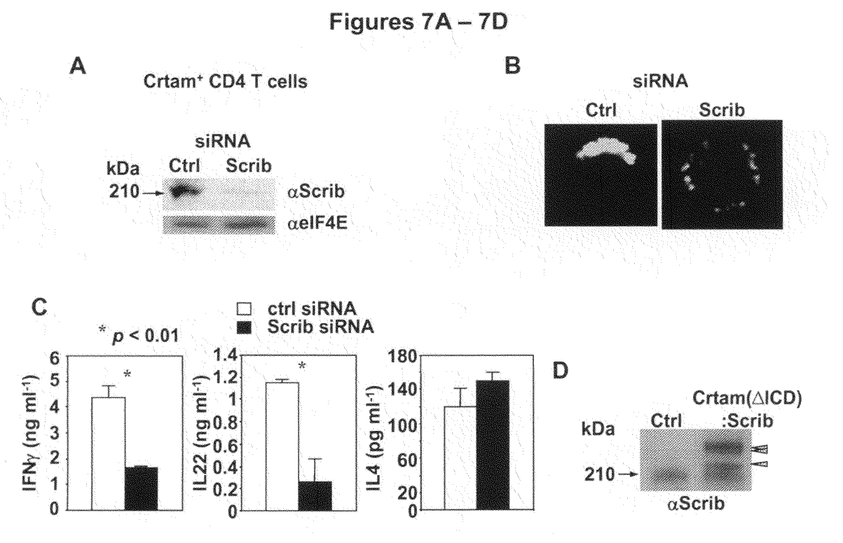

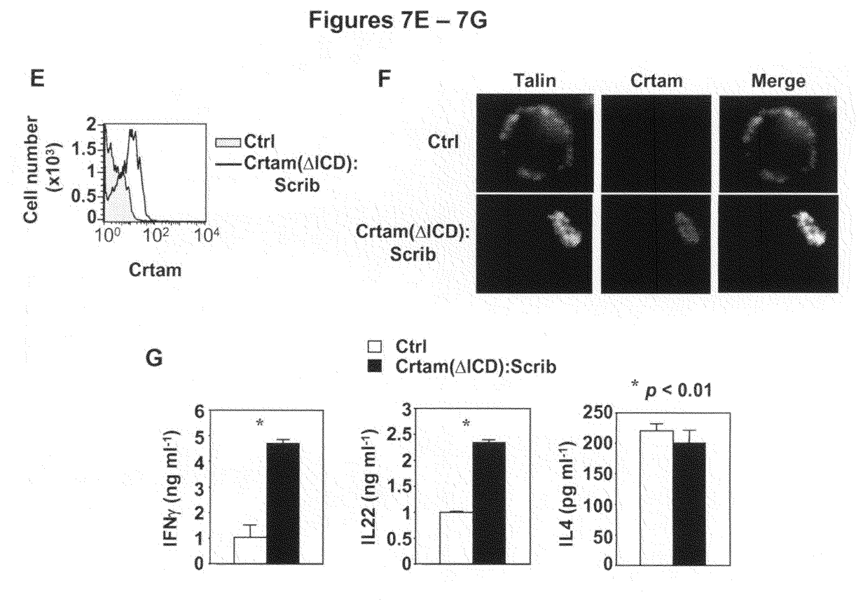

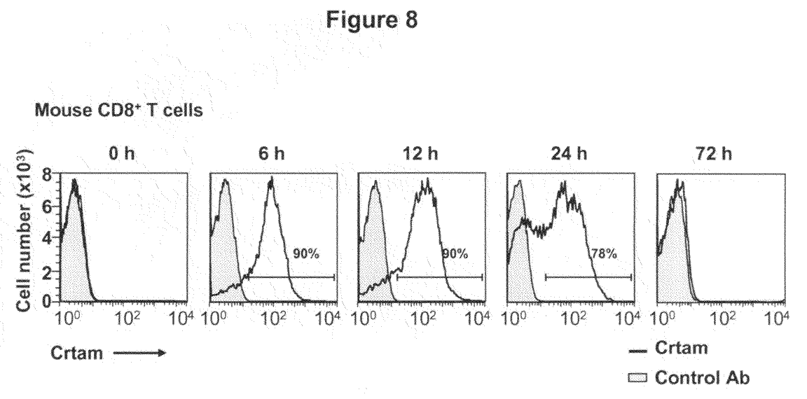

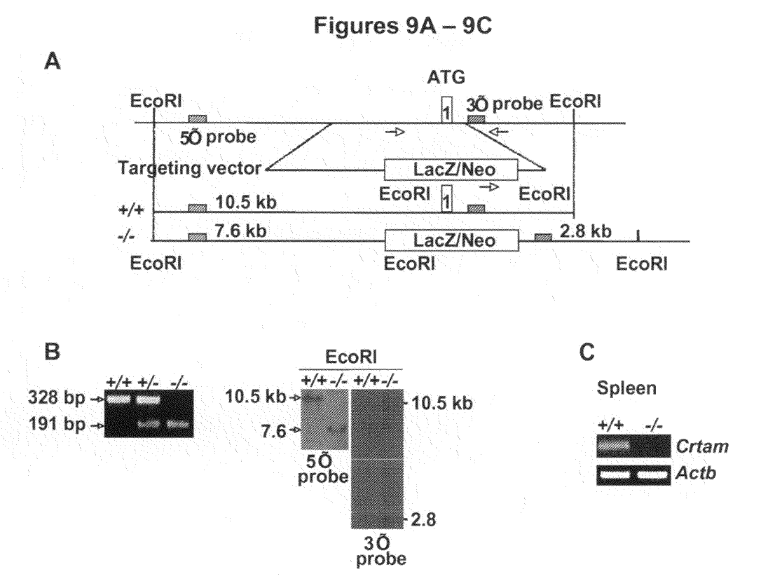

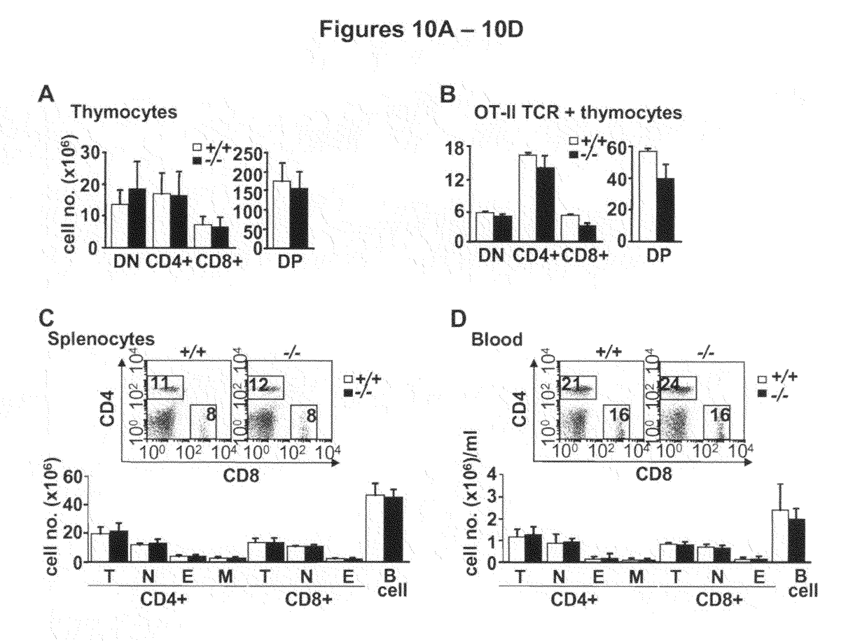

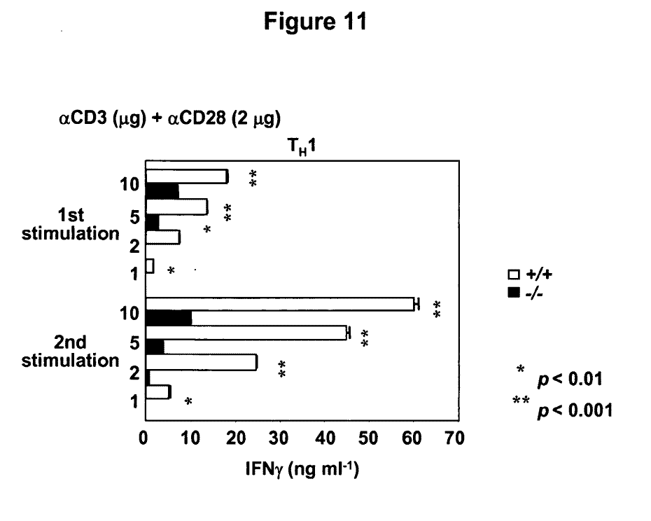

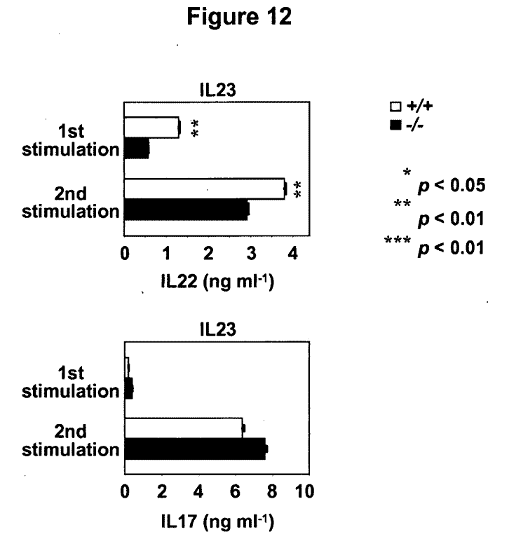

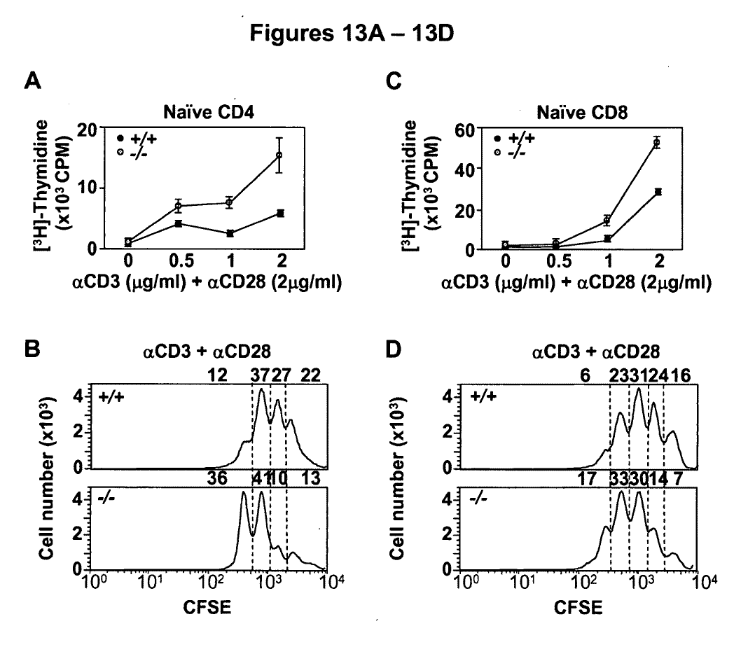

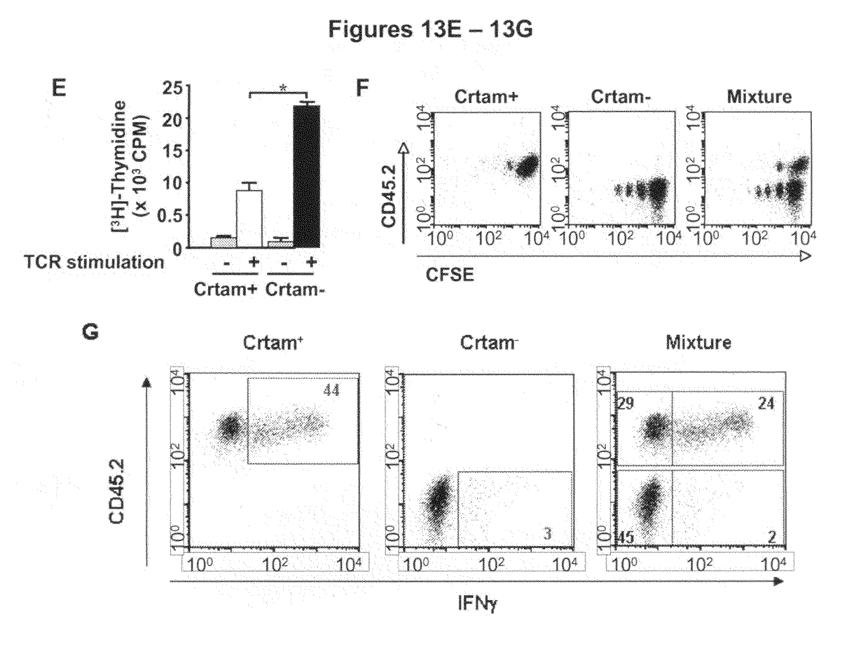

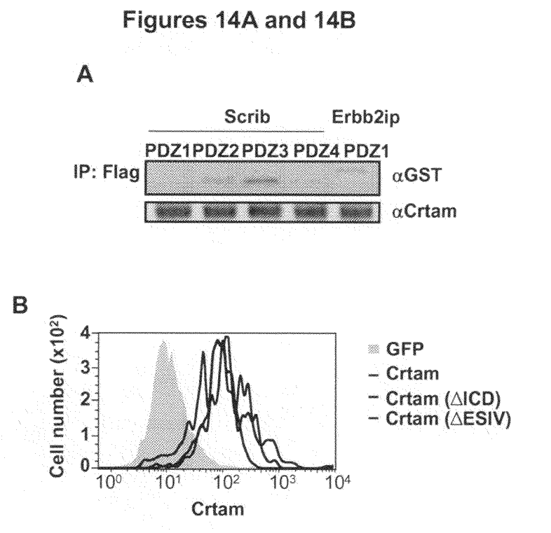

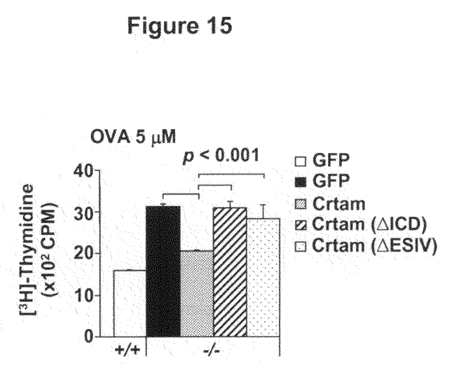

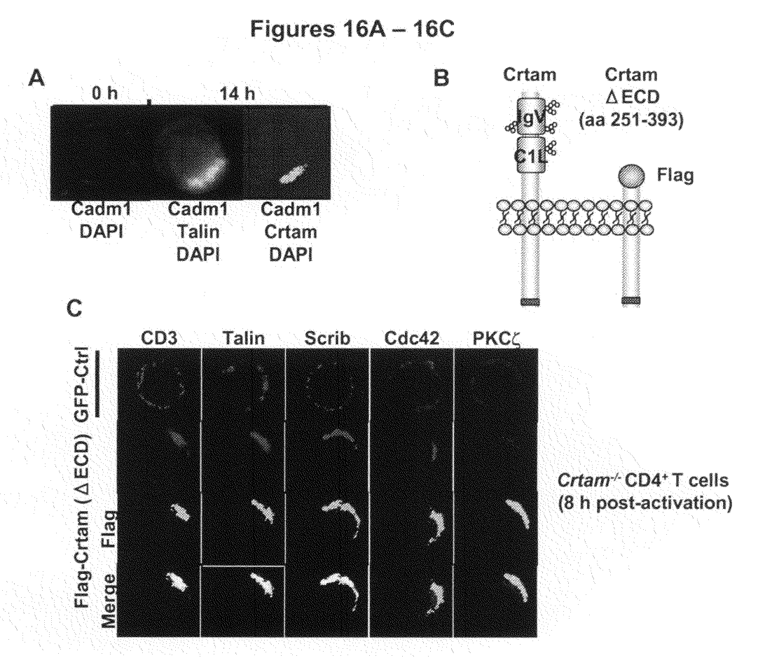

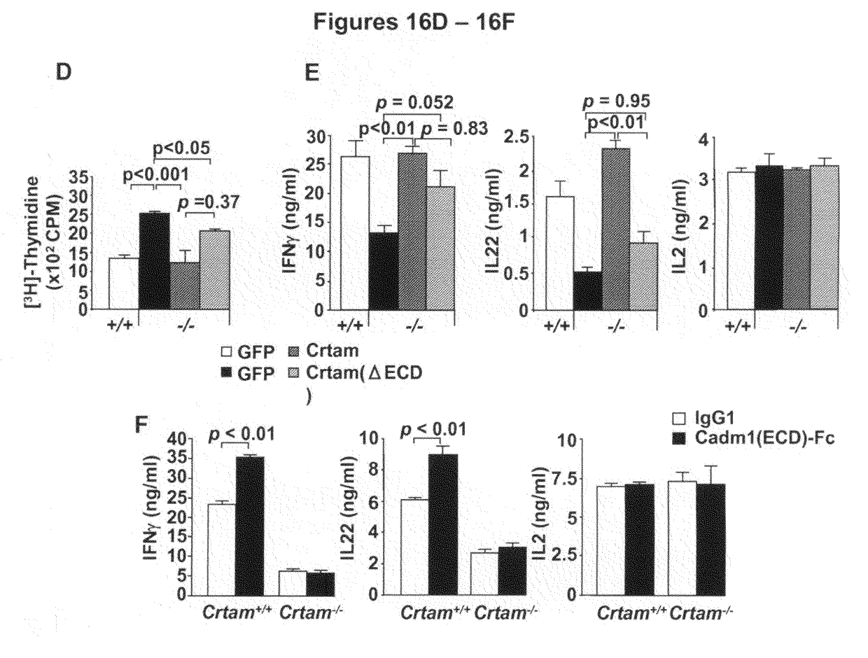

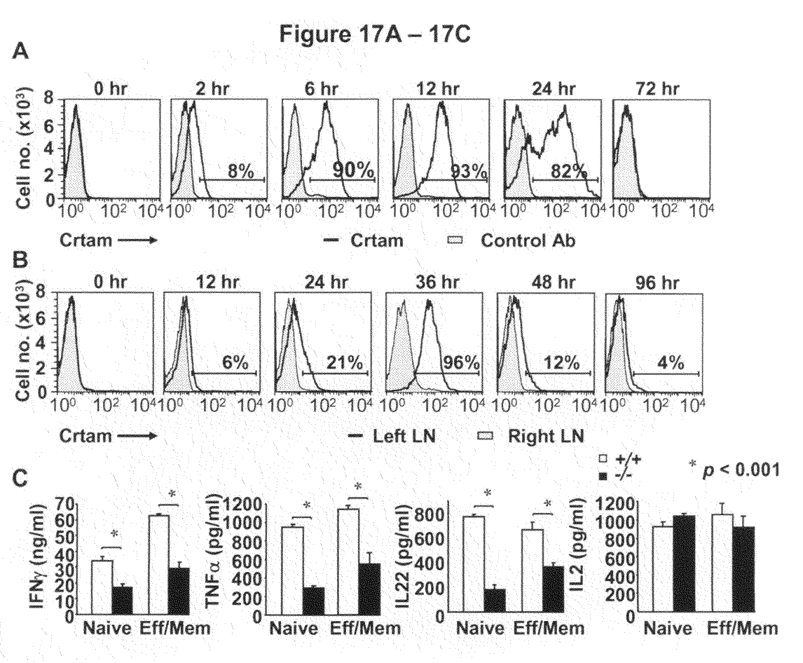

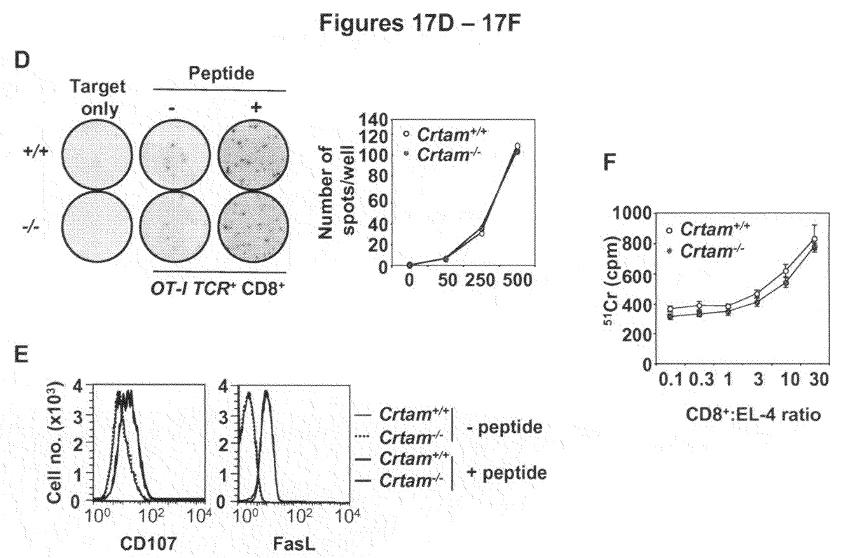

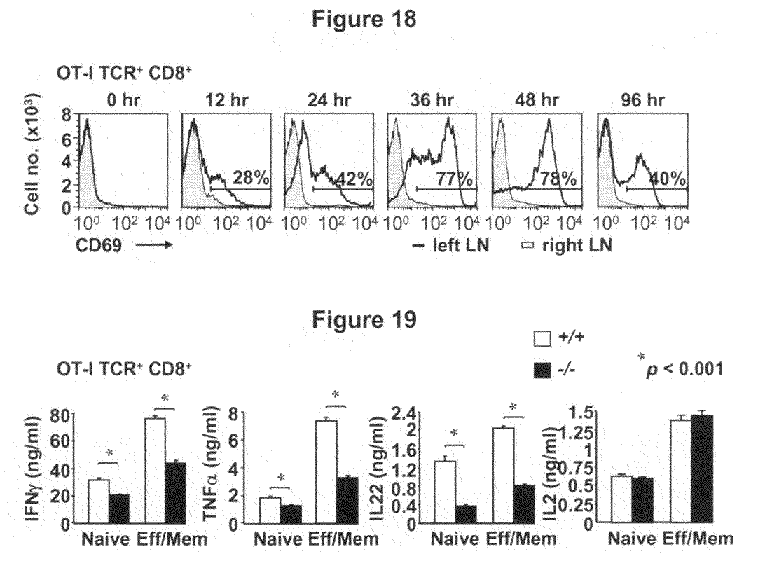

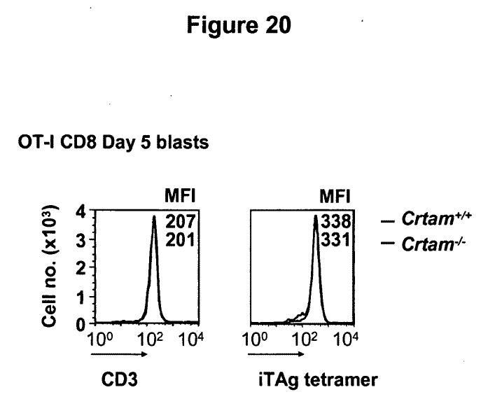

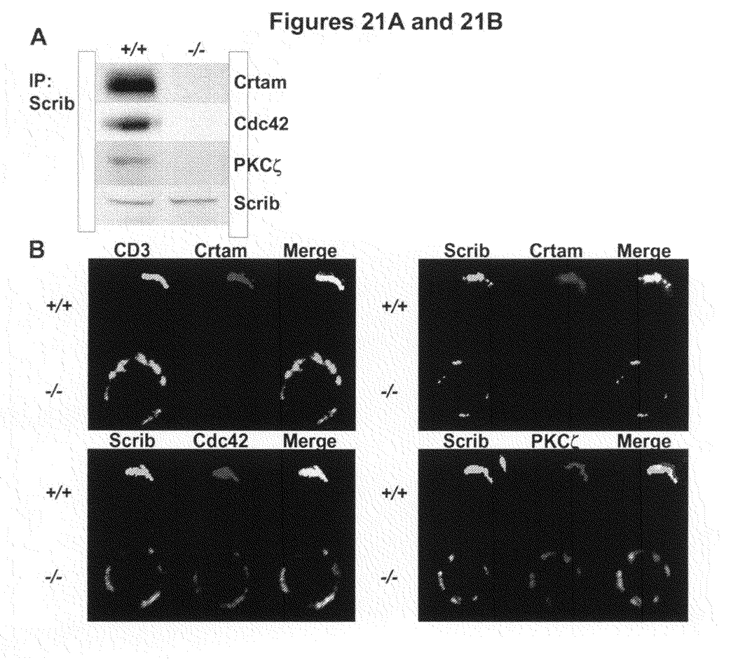

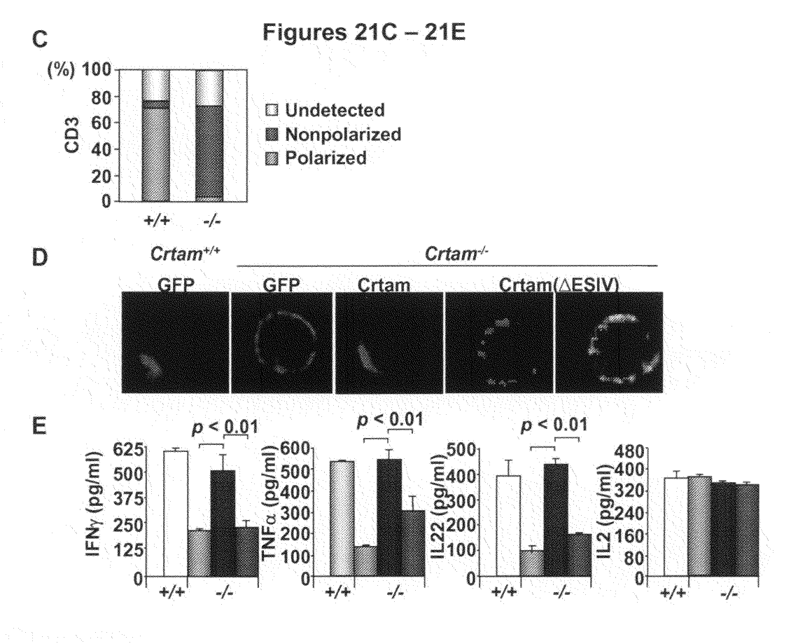

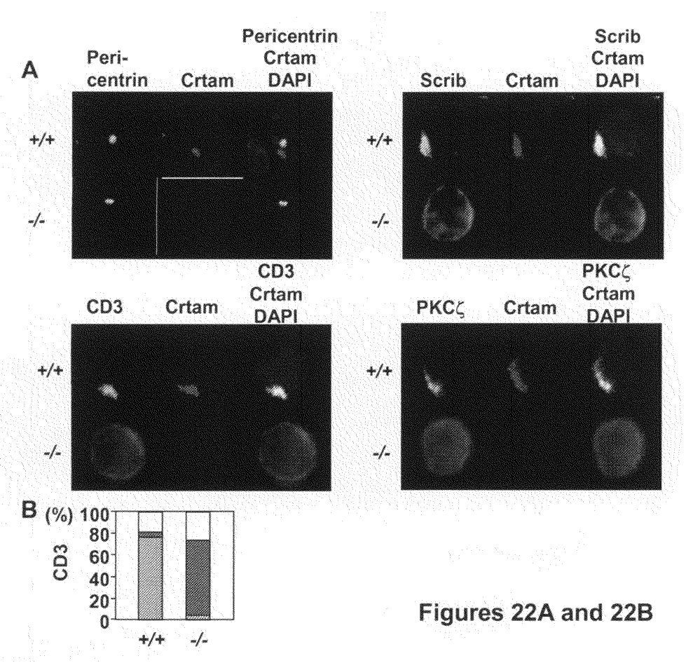

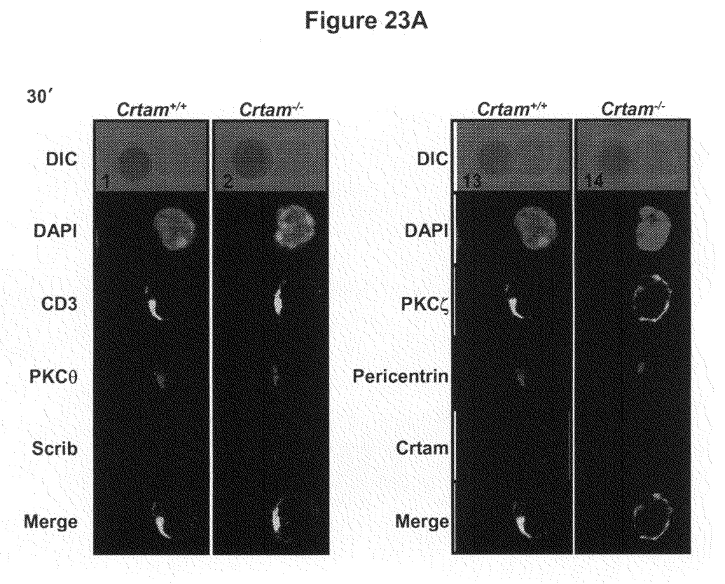

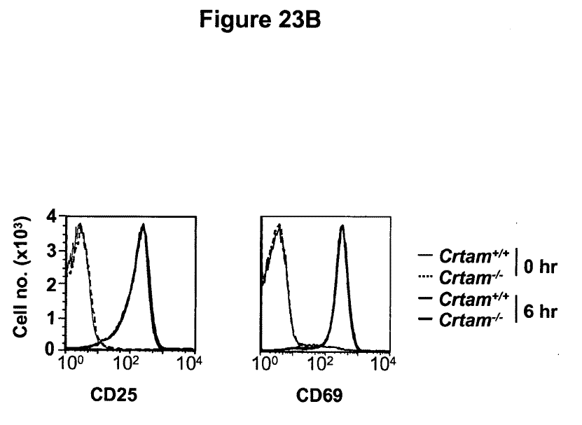

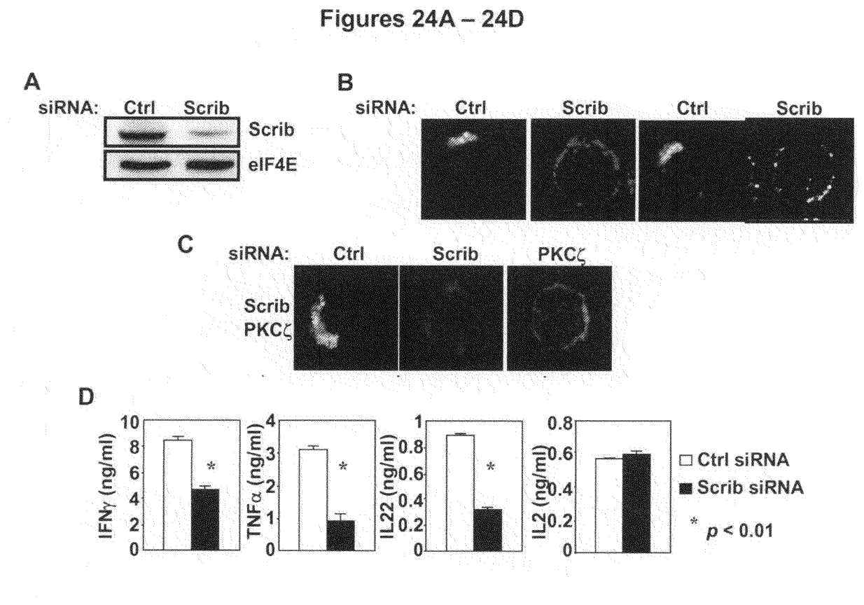

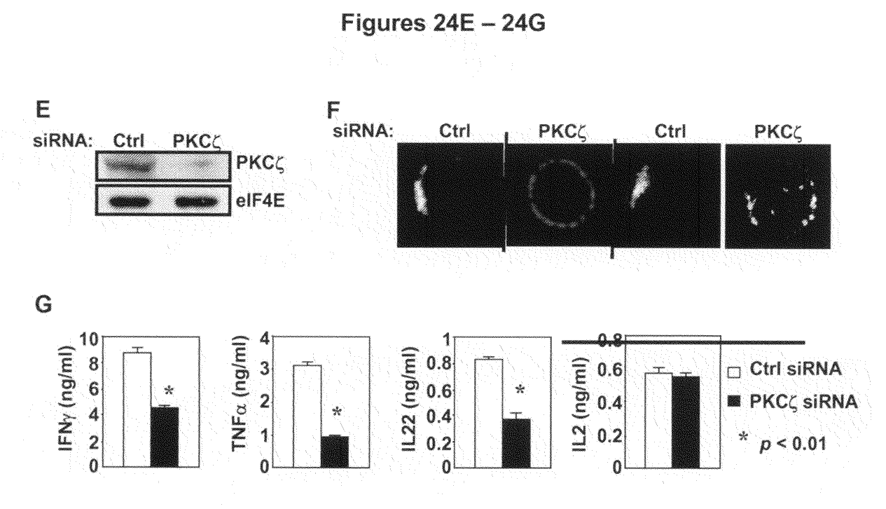

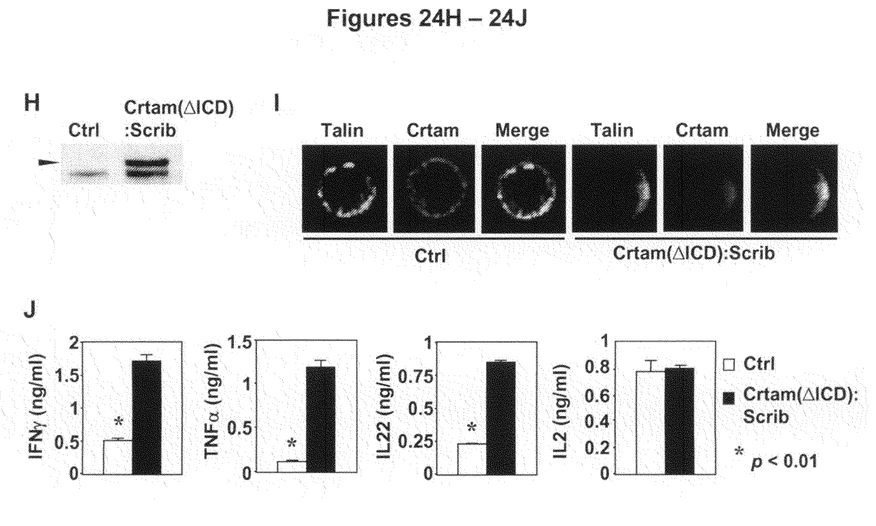

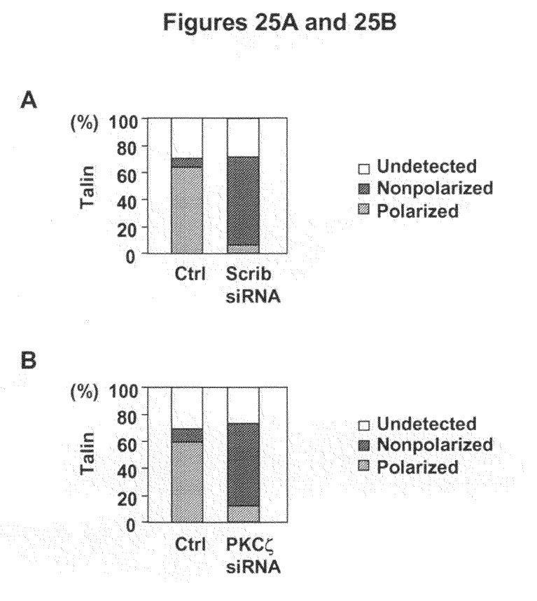

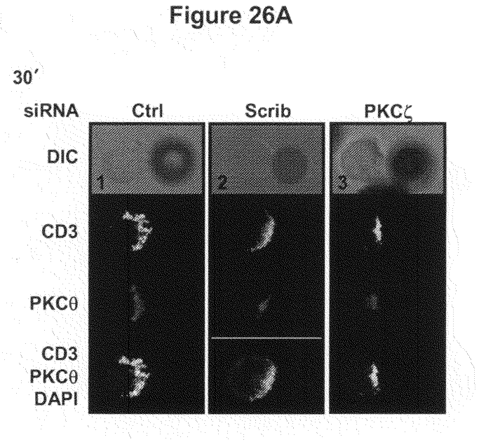

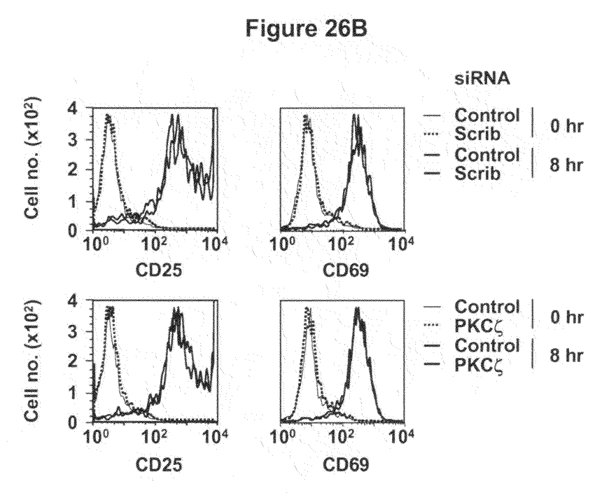

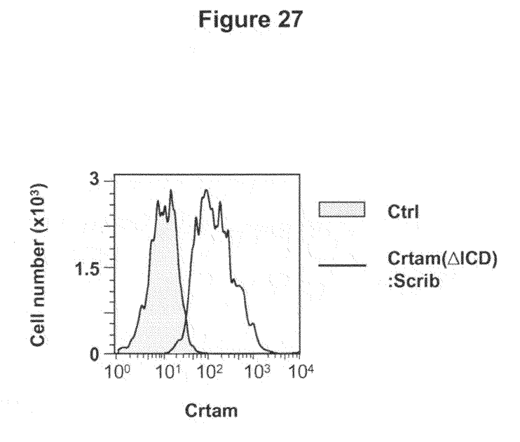

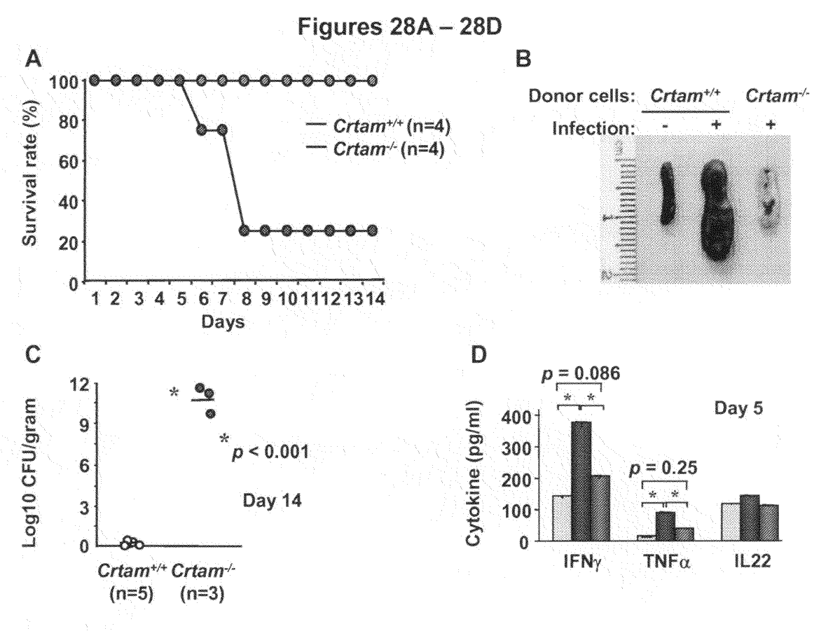

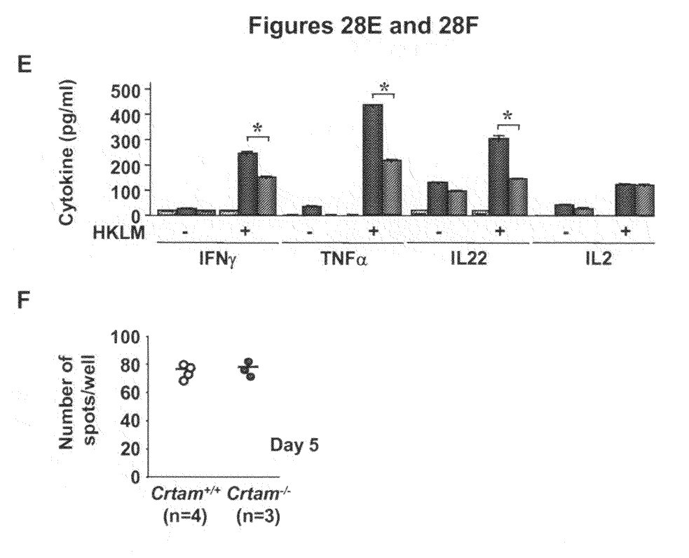

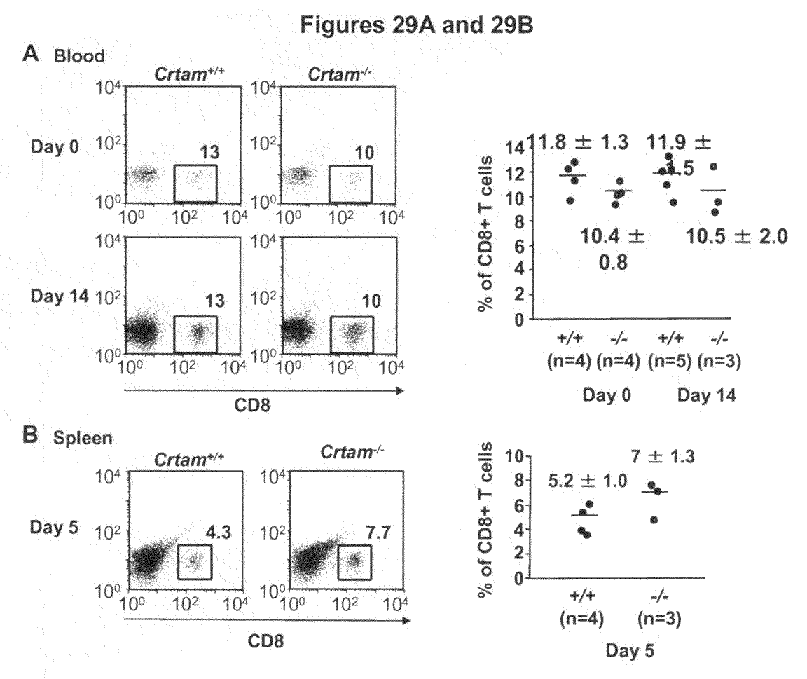

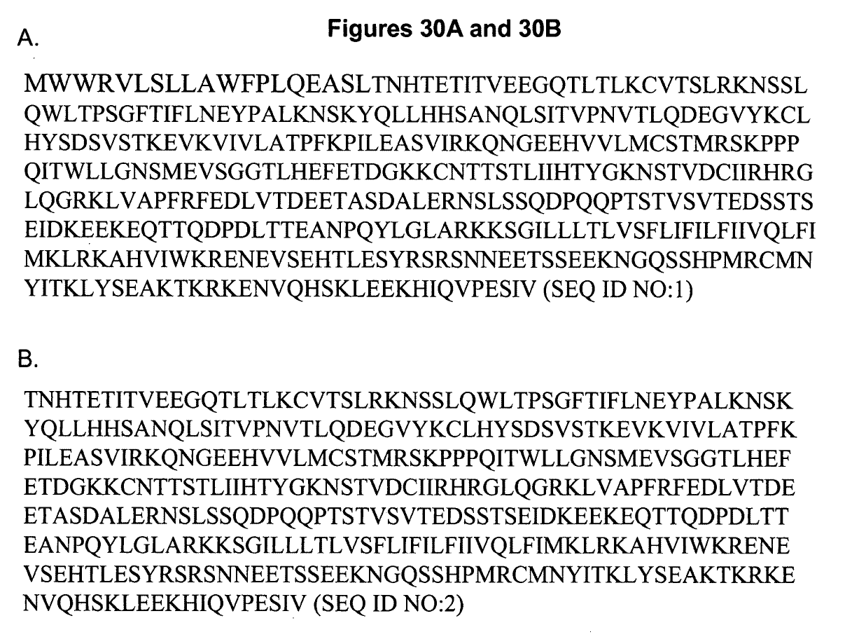

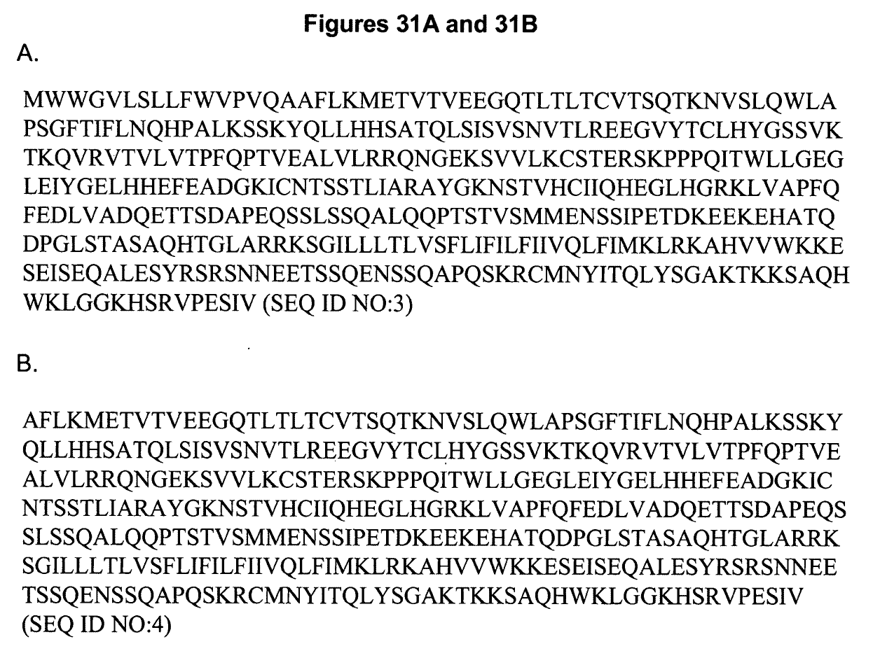

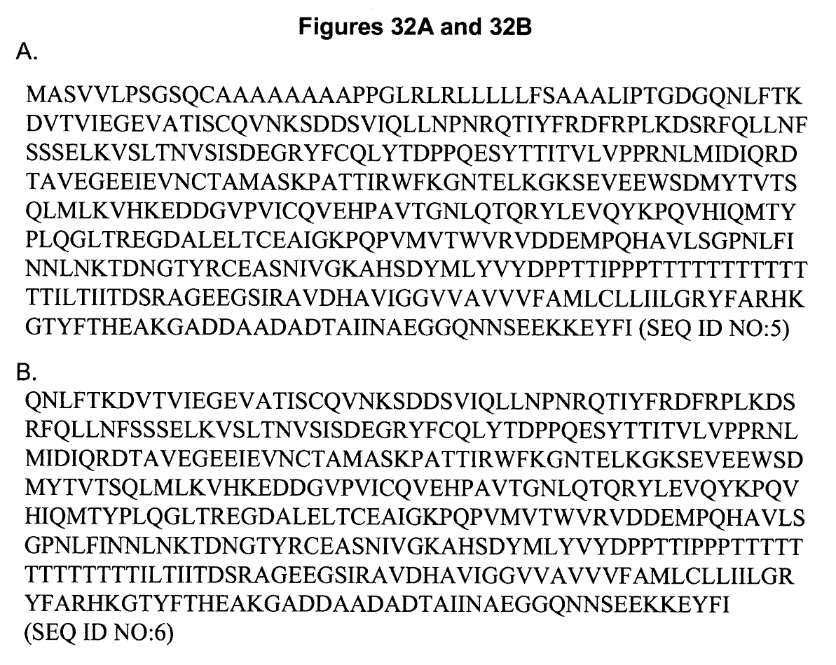

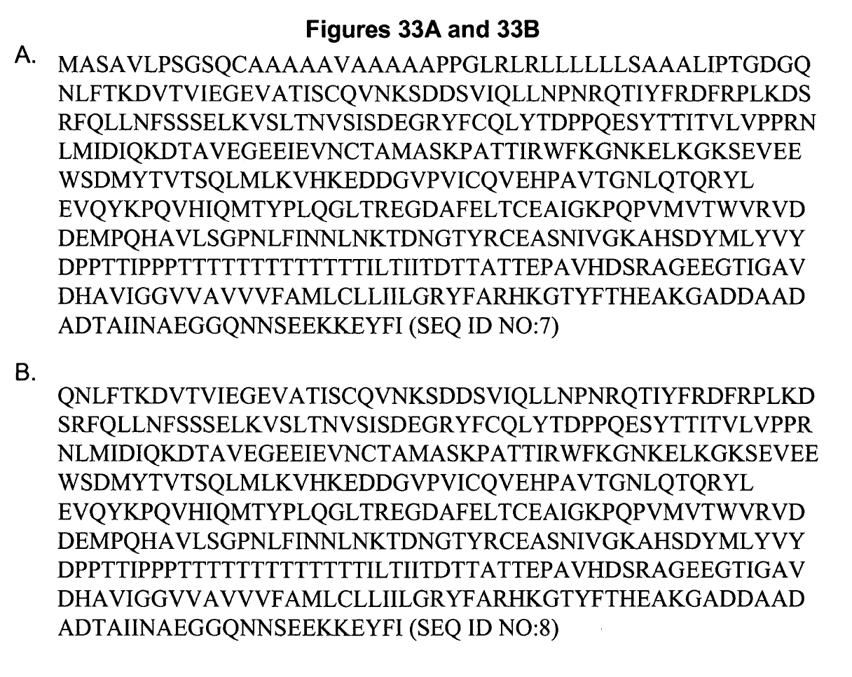

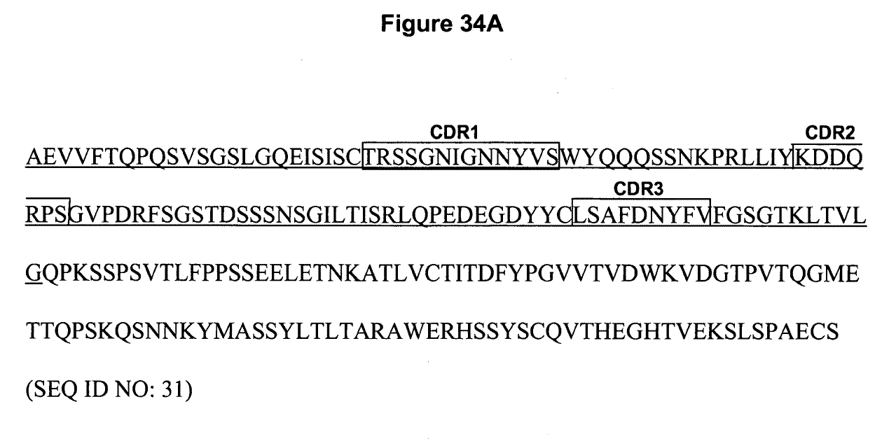

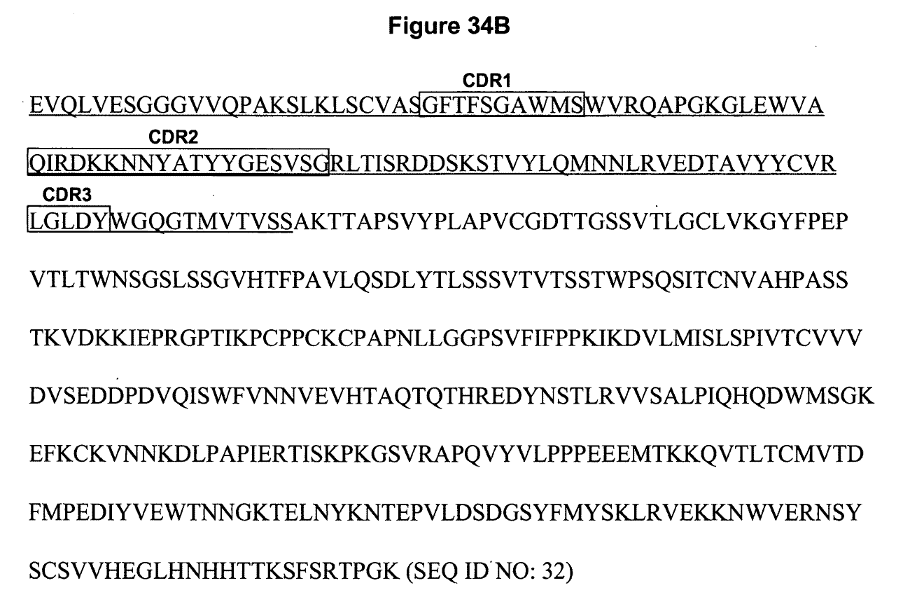

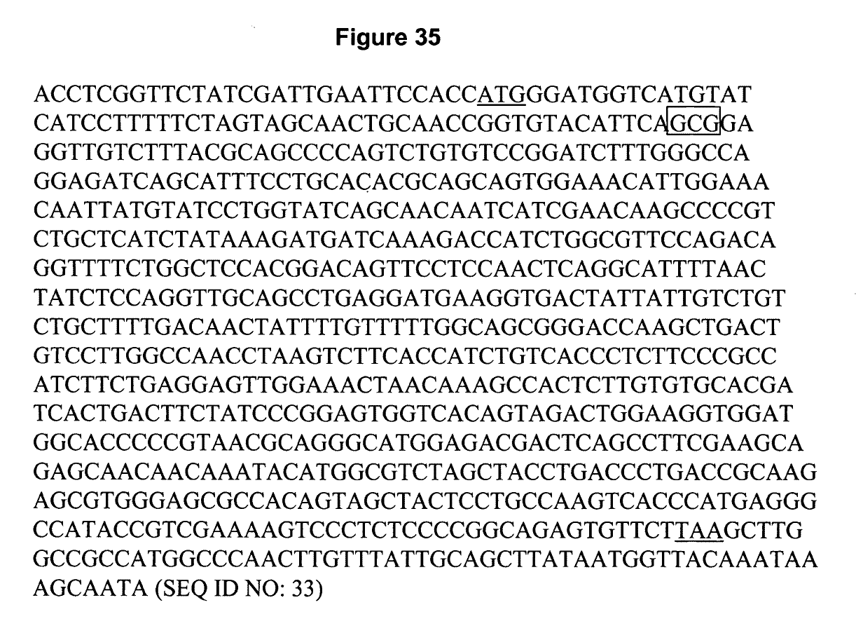

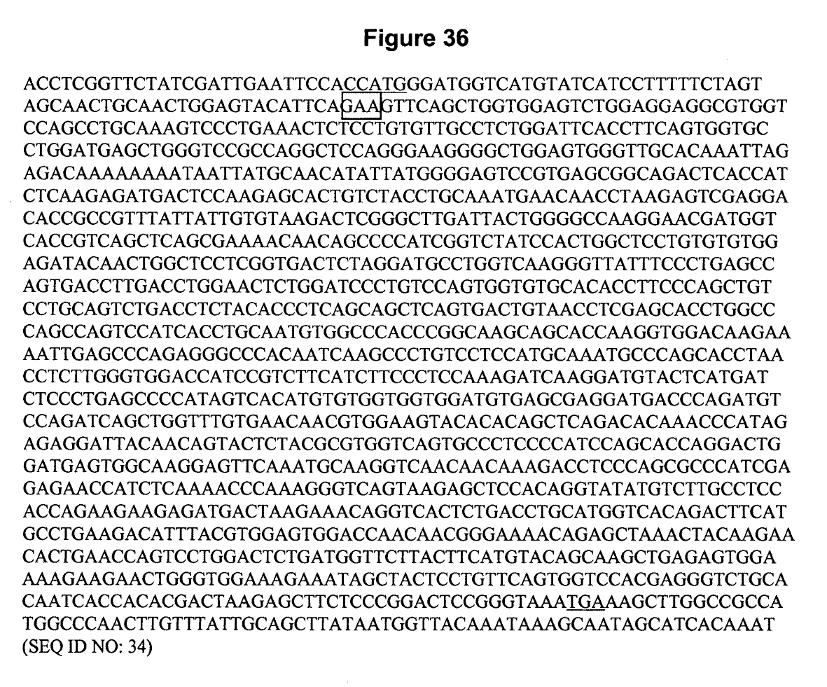

Generation of mAbs against mouse Crtam permitted us to confirm that Crtam was not expressed on resting naïve CD4⁺CD62L⁺ or CD8⁻ T cells, but was upregulated following TCR activation (FIG. 1A and FIG. 8). Expression of surface Crtam was detected in CD8⁺ T cells within 6 h following TCR activation and downregulated by 72 h. Conversely, Crtam was upregulated on a subset of CD4⁻ T cells (˜10 to 40% depending upon the mode of activation) 12 h following TCR activation and downregulated within 24 h. The kinetics of Crtam expression on OT-II TCR transgenic CD4⁺ T cells in response to peptide/APC stimulation peaked later at 24 hours as compared to anti-CD3 mediated TCR activation, but was similarly upregulated on a sub-population of CD4⁺ T cells (data not shown). The inability of Crtam to be upregulated in CD4⁻ T cells was not due to the lack of TCR activation as both Crtam⁺ and Crtam⁻ T cells expressed the CD69 activation antigen (data not shown).

Several lines of evidence suggested that the presence or absence of Crtam marked two distinct cellular populations. First, Crtam was uniformly upregulated upon TCR re-stimulation of sorted CD4⁺Crtam⁺, but not CD4⁻Crtam⁻ T cells (FIG. 1B). Second, GeneCHIP and quantitative TaqMan analysis of sorted 14 h-activated Crtam⁺ CD4⁺ T cells revealed elevated levels of IFNγ and IL22 mRNAs as compared to Crtam⁻ CD4⁺ T cells (FIG. 1C upper panels and data not shown). In contrast, levels of IL2, IL4 and IL13 mRNAs were comparable (FIG. 1C, upper panel and data not shown). Third, re-stimulation of sorted Crtam⁺ or Crtam⁻CD4⁻ T cells revealed that Crtam⁺ T cells secreted more IFNγ and IL22, but not IL2 protein than re-stimulated Crtam⁻ T cells (FIG. 1C, lower panel). As IFNγ and IL22 are associated with T_{H}1 and T_{H}17 cell functions, we further analyzed whether Crtam⁺ and Crtam⁻ cells differed under T_{H} differentiating conditions. Differentiation of CD4⁻ T cells under T_{H}1 conditions revealed that Crtam⁻ T cells secreted and produced more intracellular IFNγ (FIGS. 1D and 1E). Similarly, Crtam⁺ T cells made substantially more IL22 and IL17 when differentiated under T_{H}17 conditions (FIG. 1F). In contrast, no difference in IL4 was detected between Crtam⁺ and Crtam⁻ cells under T_{H}2 differentiation conditions (FIG. 1G). Together, these data suggest that Crtam expression on a subset of naïve CD4⁺ T cells is associated with a greater ability to synthesize and secrete IFNγ, IL22 and IL17 cytokines following TCR activation.

### Crtam^{-/-} T Cells Have Reduced IFNγ and IL22 Production.

To further investigate the physiological function of Crtam, Crtam-deficient mice were generated by homologous recombination (FIG. 9A). Gene targeting was confirmed at the DNA level (FIG. 9B), the absence of mRNA was confirmed by RT-PCR (FIG. 9C) and the lack of protein was supported by both flow cytometric and Western blot analysis of activated CD4⁻ and CD8⁺ T cells (FIGS. 2A and 2B). Crtam^{-/-} mice were born at expected Mendelian ratios. No thymic developmental defects were detected in Crtam^{-/-} mice (FIG. 10A) and positive selection of OT-II TCR transgenic thymocytes was normal (FIG. 10B). Absolute numbers of immune cells in spleen and blood from Crtam^{-/-} mice at 6-weeks of age were comparable to wild-type littermates (FIGS. 10C and 10D).

Consistent with the properties of CD4⁺ Crtam⁻ T cells isolated from wildtype mice, analysis of naïve CD4⁺ Crtam^{-/-} T cells under T_{H}1 differentiating conditions revealed decreased IFNγ mRNA levels and intracellular staining of IFNγ in TCR-activated Crtam^{-/-} T_{H}1 cells (FIGS. 2C and 2D). Reduction in IFNγ was further supported by reduced IFNγ secretion following both primary and secondary TCR stimulations when compared to CD4⁺ Crtam^{+/+} T cells (FIG. 2E and FIG. 11). Decreases in IL22, and to a lesser degree IL17, were also observed in CD4⁺ Crtam^{-/-} T cells under T_{H}17 commitment conditions (TGFβ and IL6) (FIG. 2E). A more modest reduction in IL22, but not IL17, was observed when cells were incubated with IL23, a condition that preferentially induces IL22 during primary T cell activation (Zheng et al., 2007) (FIG. 12). In contrast, no difference in IL4 production was observed in Crtam^{-/-} T cells differentiated under T_{H}2 conditions (FIG. 2E). Hence, loss of Crtam resulted in decreased secretion of IFNγ (a T_{H}1-associated cytokine) as well as IL22 and IL17 (T_{H}17-associated cytokines). Similarly, TCR activation resulted in decreased IFNγ secretion in CD8⁺ Crtam^{-/-} naïve and effector/memory populations (FIG. 2F). As both CD4⁺ T cells and IFNγ are required for host defense against Citrobacter rodentium, a non-invasive attaching and effacing bacteria (Eckmann, 2006; Mundy et al., 2005), we orally inoculated Crtam^{+/+} and Crtam^{-/-} mice with C. rodentium and analyzed bacterial burden 7 and 14 days post-infection. Consistent with the in vitro defects of Crtam^{-/-} T_{H}1 cells, Crtam^{-/-} mice demonstrated >1 log increase in colonic bacterial burden on day 7 and ˜2 log increase in splenic pathogen burden at 7 and 14 days post-infection (FIG. 2G). Together, these data indicate that Crtam deficiency is associated with a selective defect in IFNγ, IL22 and IL17 production and compromised host resistance to oral C. rodentium infection.

### Crtam^{-/-} T Cells Demonstrate Increased TCR-Mediated Proliferation

In contrast to decreased IFNγ and IL22 secretion, naïve CD4⁺CD62L⁺ Crtam^{-/-} T cells expressing an MHC class II-restricted OT-II TCR displayed increased [³H]-thymidine incorporation when stimulated with irradiated APCs pulsed with OVA peptides when compared to Crtam^{+/+} T cells (FIG. 3A). Enhanced cellular proliferation was further supported by increased dilution of CFSE-labeled Crtam^{-/-} CD4⁺ T cells following antigen challenge (FIG. 3B). Similar results were obtained with OT-I TCR⁺ CD8⁺ T cells in response to peptide/APC stimulation (FIGS. 3C and 3D) and when CD4⁺ and CD8⁺ T cells were stimulated with anti-CD3 and CD28 mAbs (FIGS. 13A-D). Analysis of Crtam⁺ and Crtam⁻ cells revealed similar results. Sorted Crtam⁺ T cells upon TCR re-stimulation demonstrated increased [³H]-thymidine incorporation and increased CFSE dilution when compared to sorted Crtam⁺ T cells (FIGS. 13E and 13F). To ensure that differences in proliferation between Crtam⁺ and Crtam⁻ T cells were not due to extrinsic factors, such as the indirect anti-proliferative effects of IFNγ (Gajewski and Fitch, 1988), CFSE-labeled CD45.2⁺ Crtam⁺ and CD45.2⁻ Crtam⁻ T cells were mixed and activated together with anti-CD3 and CD28 mAbs. Despite co-incubation, Crtam⁻ T cells still proliferated faster and synthesized less IFNγ than Crtam⁺ T cells (FIGS. 13F and 13G). Hence, differences in proliferation between Crtam⁺ and Crtam⁻ T cells were due to the direct intrinsic function of Crtam.

The in vitro hyperproliferation was also observed in vivo as adoptively transferred CFSE-labeled Crtam^{-/-} OT-II TCR⁺ CD4⁺ T cells demonstrated increased cell divisions in response to OVA immunization when compared to Crtam^{+/+} OT-II TCR CD4⁺ T cells (FIG. 3E). In turn, older Crtam^{-/-} mice accumulated CD4⁺ and CD8⁺ T, but not B, cells in the peripheral lymph nodes and blood when compared to their wildtype littermates (FIG. 3F). Together, these data indicate that Crtam^{-/-} CD4⁺ and CD8⁺ T cells demonstrate increased cycling and proliferation in response to TCR activation.

### Crtam Controls a Late Phase of T Cell Polarity.

Biochemical analysis of TCR-mediated signaling pathways of Crtam^{+/+} and Crtam^{-/-} T cells revealed that loss of Crtam did not impact signaling processes within the initial phases of T cell activation during which Crtam is not expressed, but likely impacts cellular processes with its upregulation within the 6 to 18 hour timeframe following TCR engagement (data not shown). Analysis of naïve CD4⁺ T cells within this latter phase of T cell activation revealed that Crtam, Talin and CD3 were co-localized and asymmetrically polarized relative to CD44 14 h following T cell activation (FIG. 4A, panels 4-6; FIG. 4B, panels 3-4; FIG. 4C, panels 4-6). This asymmetric polarization of CD3/Talin and CD44 was dependent upon Crtam. In Crtam^{-/-} T cells, CD3, Talin and CD44 were detected as multiple non-polarized focal aggregates 14 hours following co-crosslinking of anti-CD3 and anti-CD28 mAbs (FIG. 4A, panel 11, FIG. 4B, panel 7 and FIG. 4C, panel 11). Quantitation of >200 cells per condition demonstrated the inability of Crtam^{-/-} T cells to efficiently polarize Talin, CD44 and CD3 during this latter phase of T cell activation (FIG. 4D). Construction of a 3-dimensional model by reconstitution of 2-dimensional slat images further established the focal co-localization of Crtam/Talin (FIG. 4E, panel 3) and asymmetric polarization of CD44 (panel 4) in Crtam^{+/+} T cells as well as the loss of this asymmetric polarity of Talin (panel 5) and CD44 (panel 6) in Crtam^{-/-} T cells following TCR activation. Similar defects in Talin (FIG. 4F, panel 4), CD44 (panel 5) and CD3 (panel 6) polarization were observed in naïve OT-II TCR⁺ Crtam^{-/-} T cells 14 h post-activation using OVA peptide pulsed APCs (FIG. 4G). In contrast to the defects observed during this latter phase of T cell activation, OT-II TCR⁺ CD4⁺ Crtam^{-/-} T cells were capable of interacting within the first 30 minutes with OVA antigen pulsed DCs as assessed by confocal microscopy and by a FACS-based conjugation assay (FIGS. 4H and 4I). Hence, consistent with the kinetics of its upregulation between 6 and 12 hours following TCR activation, Crtam does not play any role during the initial phases of T cell:APC conjugate formation, actin polymerization, reorganization of the microtubule organizing center (MTOC) or early TCR-mediated signaling, but is required for establishing T cell polarity during the latter phases of T cell activation.

### Crtam Assembles a Scrib-Containing Complex to Control Late T Cell Polarity and Cytokine Production.

The studies of the PDZ domains of human Scrib have revealed a conserved C-terminal recognition motif ([D/E][T/S]X[LIV]_{COOH}), which is found in many diverse proteins including Crtam (ESIV_{COOH}) that may interact with Scrib (Tonikian et al., 2007). To define the mechanism(s) by which Crtam regulates T cell morphology, we analyzed the ability of Scrib and Dlg1 to co-immunoprecipitate with Crtam in resting and activated T cells (FIG. 5A). Crtam was detected in Scrib, but not Dlg1, immunoprecipitates 14 h following TCR activation. This interaction was direct as the third PDZ domain within Scrib was sufficient to interact with Crtam (FIG. 14A). Confocal microscopy revealed that Scrib co-localized with Crtam and CD3 during this late phase following TCR activation (FIG. 5B, panels 1-3 and data not shown). As Scrib controls polarization of astrocytes through a Cdc42 GTPase/β-PIX guanine nucleotide exchange factor dependent pathway (Audebert et al., 2004; Osmani et al., 2006), we analyzed whether Crtam/Scrib controlled Cdc42 and PKCζ localization. In naïve CD4⁺ T cells, TCR activation resulted in polarized co-localization of Crtam, Scrib, Cdc42 and PKCζ (FIGS. 5B-D, panels 1-3). Conversely, absence of Crtam resulted in loss of Scrib and Cdc42 polarization and co-localization (FIGS. 5B-D, panels 4-6). Together, our studies suggested the existence of distinct signaling machinery that controlled early and late phases of T cell polarity.

To determine if similar or distinct molecular complexes may be involved in regulating the early and late phases of T cell polarity, we analyzed the localization of CD3, Talin and CD44 and compared the subcellular localization of molecules implicated during the early phases of T cell activation (PKCθ) and the Crtam/PKCζ complex using OTII TCR⁺CD4⁺ T cells when presented with OVA₃₂₃₋₃₃₉ pulsed APCs (FIG. 5E). While CD3, Talin and CD44 maintained their capped structures during the entire 12 h period following T cell activation (FIG. 5E, first three columns), distinct modules of signaling proteins were co-localized with CD3 during the early (<2 h) and late (>6 h) phases following TCR engagement. Consistent with previous reports, PKCθ was rapidly co-localized with CD3 (panels 25, 32 and 39) (Monks et al., 1997), but was lost by 4 hours (panel 46) following T cell activation. In comparison, while PKCζ was not co-localized with CD3 within the first 4 hours following T cell activation (panels 12, 19, 26, 33, 40 and 47), the appearance of Crtam resulted in the co-localization of PKCζ with Crtam 8 h following T cell activation (panel 54). Hence, distinct modules of signaling proteins are co-localized with CD3 and Talin during the early and later phases of T cell activation.

### Requirement for Crtam:Scrib Interaction in Late T Cell Polarity and IFNγ/IL22 Production.

To define the structural basis for the requirement of Crtam in Scrib polarization and enhanced cellular proliferation, we utilized retroviral transduction of Crtam, Crtam lacking the entire intracellular domain (ΔICD) or Crtam lacking the C-terminal four amino acids required for its interaction with Scrib (ΔESIV) in Crtam^{-/-} or OT-II TCR⁺ CD4⁺ Crtam^{-/-} T cells (FIG. 6A). While Crtam reversed the enhanced proliferation observed with Crtam^{-/-} T cells, neither Crtam (ΔICD) nor Crtam (ΔESIV) reversed the hyperproliferative phenotype despite comparable levels of protein reconstitution (FIG. 6B, FIGS. 14B and 15). These data are consistent with the increased cellular proliferation observed with loss of Scrib in Drosophila and epithelial cells (Bilder et al., 2000; Nagasaka et al., 2006). Similar to the proliferative phenotype, only full length Crtam restored IFNγ production, IL22 production and Talin polarization (FIGS. 6C and 6D). Neither Crtam (ΔICD) nor Crtam (ΔESIV) restored the cytokine defects or Talin polarization. Additionally, expression of Crtam, Crtam(ΔICD) or Crtam(ΔESIV) had no effect on IL4 production. Thus, up-regulation of Crtam following T cell activation regulates T cell polarity through its interaction with Scrib that, in turn, controls cellular proliferation and selective cytokine production.

To determine whether expression of Crtam alone was sufficient for IFNγ and IL22 secretion, we sorted naïve CD4⁺CD62L⁺Crtam⁻T cells from wildtype mice and retrovirally transduced Crtam, Crtam (ΔESIV) or a control vector. Expression of wild type Crtam in sorted Crtam⁻ T cells from wildtype mice was sufficient to confer IFNγ and IL22, but not IL4, production to levels comparable found in sorted Crtam⁺ T cells (FIG. 6E). This gain of function was dependent upon the ability of Crtam to interact with Scrib, as Crtam (ΔESIV) was unable to confer IFNγ or IL22 production. Coincident with gain of IFNγ and IL22 production, expression of wildtype Crtam in sorted Crtam⁻ T cells also resulted in Talin polarization (FIG. 6F), an effect that also required its interaction with Scrib (panel 4). Hence, expression of Crtam can induce Crtam⁻ T cells, isolated from wildtype mice, to control cellular proliferation, establish cellular polarity and specifically induce IFNγ and IL22 production.

To further establish the importance of Scrib in Crtam function, we introduced Scrib siRNA into FACS sorted Crtam⁺ CD4⁺ T cells from C57/BL6 mice. Decrease in Scrib protein was confirmed by Western blotting (FIG. 7A). Knockdown of Scrib protein resulted in loss of Talin polarization 8 hours following TCR activation (FIG. 7B) as well as inhibition of IFNγ and IL22, but not IL4, production following TCR activation (FIG. 7C). Finally, to establish the importance of the Crtam-Scrib interaction, we expressed a chimeric receptor, designated as Crtam(ΔICD):Scrib, consisting of the extracellular and transmembrane domains of Crtam lacking its native ICD, but fused to a cytoplasmic domain consisting of Scrib. Expression of Crtam(ΔICD):Scrib in Crtam^{-/-} T cells was confirmed by Western blot analysis (FIG. 7D) and FACS staining (FIG. 7E). Expression of Crtam(ΔICD):Scrib restored Talin polarization (FIG. 7F, panel 5) and selectively increased IFNγ and IL22, but not IL4, production (FIG. 7G). Collectively, these results underscore the importance of the Crtam:Scrib interaction to regulate this late phase of T cell cytoskeletal polarity and selective cytokine production.

### Extracellular Domain of Crtam Regulates Cytokine Production

While the interaction of Scrib with the ICD of Crtam was critical for Crtam function, we also assessed the contribution of the extracellular domain (ECD) of Crtam. Crtam binds cell adhesion molecule (Cadm1, also known as Necl2) that, in turn, can bind itself through homotypic interactions and Cadm3/Necl1 through heterotypic interactions (Galibert et al., 2005; Shingai et al., 2003). In contrast to Crtam, Cadm1 was expressed at low levels on resting naive T cells and its expression was further downregulated 14 h following TCR activation (data not shown). Moreover, confocal microscopy revealed that Cadm1 was diffusely distributed on both resting and activated T cells and did not co-localize with either Talin or Crtam 14 hours following TCR activation (FIG. 16A). To assess the contribution of the ECD of Crtam, we expressed a FLAG-epitope tagged mutant of Crtam lacking the ECD, but encoding the transmembrane and intracellular domains of Crtam (aa 251-393) (FIG. 16B). Despite lacking its ECD, expression of Crtam(ΔECD) in Crtam^{-/-} T cells retained its ability to polarize with CD3, Talin, Scrib, Cdc42 and PKCζ 8 hours following TCR activation (FIG. 16C, panels 16-20). In addition, Crtam(ΔECD) partially reversed the TCR-mediated hyperproliferative phenotype observed in Crtam^{-/-} T cells (FIG. 16D). Surprisingly, expression of Crtam(ΔECD) was unable to fully restore IFNγ or IL22 production (FIG. 16E). Hence, the ECD of Crtam appeared to be dispensable for control of normal cellular division and establishment of a late phase of T cell polarity, but contribute to IFNγ or IL22 production.

To further explore the contributions of the ECD of Crtam in cytokine production, Crtam^{+/-} naive T cells were activated with anti-CD3 and anti-CD28 mAbs in the absence or presence of a Cadm1 (ECD)-Fc fusion protein encoding the ECD of Cadm1 (aa 1-374) fused to the Fc-domain of human IgG (FIG. 16F). Co-incubation of Cadm1(ECD)-Fc augmented IFNγ and IL22, but not IL2, production in TCR-activated Crtam^{+/+} T cells. In contrast, Cadm1(ECD)-Fc had no effect on cytokine production on Crtam^{-/-} T cells. Since expression of Cadm1 and Cadm3 was not affected in Crtam^{-/-} T cells (data not shown), augmentation of IFNγ and IL22 production by Cadm1(ECD)-Fc in Crtam^{+/+} T cells is likely to be mediated by Cadm1:Crtam interactions. Collectively, these data distinguish functional contributions by the ligand binding of Cadm1 to the ECD of Crtam in the selective regulation of IFNγ and IL22 production and the contributions of the transmembrane and IC domains of Crtam in establishing cellular polarity and the control of cell division.

### Crtam is Uniformly Upregulated in Activated CD8+ T Cells

Establishment of cellular polarity is requisite in asymmetric cellular division, cell migration, organ development and tissue morphogenesis. Activated CD8⁺ cytotoxic T lymphocytes (CTLs) play important roles in tumor eradication and clearance of intracellular pathogens through their cytolytic programs and secretion of pro-inflammatory cytokines Encounter with naïve CD8+ T cells with antigen initiates T cell antigen receptor (TCR) activation that is associated with an orchestrated temporal and spatial reorganization of cellular proteins required for cellular proliferation and differentiation into cytotoxic T lymphocytes (CTLs). As discussed below, we demonstrate here that activation of CD8⁺ T cells result in upregulation of Crtam (Class I restricted T cell associated molecule) to organize a Scribble containing signaling complex that maintains a late phase of T cell polarity important in anti-Listeria immunity.

As in CD4 T cells, CD8⁺ T cells organize an immunological synapse (IS) at the TCR contact site and segregate CD3ζ, Lck and PKCθ within the central supramolecular activation complex (cSMAC) that is spatially separated from an adhesion ring containing CD11a and talin residing in the peripheral SMAC (pSMAC). Upon target cell recognition, there is rapid polarization of microtubule organizing center (MTOC) and Golgi apparatus toward the target cell. MTOC reorientation facilitates movement of the lytic granules along microtubules to the contact site and focuses release of lysosomal content, including perforin and granzyme B, to a specific site between the signaling molecule patch and the adhesion ring. Uptake of stored effector mediators into the cytosol of the target cell results in the eventual target cell death through a caspase-dependent pathway. Hence, cytolytic activity requires appropriate spatial and temporal reorganization of cellular proteins in CD8⁺ T cells.

As discussed above, we have demonstrated an important role for Crtam in maintaining T cell polarity in a subset of naïve CD4⁺ T cells to selectively augment IFNγ, IL17 and IL22 production. In contrast to naïve CD4⁺ T cells, naïve CD8⁺ T cells uniformly upregulate Crtam following T cell antigen receptor activation. While Crtam was not expressed in naïve CD8⁺ T cells as measured by RT-PCR and FACS analysis (FIG. 17A), co-crosslinking with plate-bound anti-CD3/28 mAbs resulted in Crtam upregulation within 2 hours, peaked at 6-12 hours and was absent by 72 hours in naïve CD8⁺ T cells (FIG. 17A). Challenge of OT-1 TCR transgenic mice with ovalbumin antigen in the left footpad similarly resulted in upregulation of Crtam on CD8⁻ T cells isolated from the left, but not from the right, popliteal lymph node (FIG. 17B). The in vivo kinetics of upregulation of Crtam was different than CD69 as maximal upregulation of Crtam was achieved by 36 hours and absent by 96 h while maximal upregulation of CD69 was achieved at 48 hours and still maintained at 96 h (FIG. 18). Hence, Crtam is an early activation antigen on naïve CD8⁻ T cells.

While we have demonstrated that naïve and effector/memory CD8⁺ Crtam^{-/-} T cells had reduced IFNγ production, these cells also demonstrated reduced TNFα and IL22 production following TCR engagement with anti-CD3/28 mAbs or OVA pulsed APCs (FIGS. 17C and 19). Regulation was at a transcriptional level as both IFNγ and TNFα mRNA levels were also decreased in TCR-activated Crtam^{-/-}, as compared to Crtam^{-/+} CD8⁺ T cells (data not shown). In contrast, no difference in IL2 production was detected between Crtam^{+/+} and Crtam^{-/-} CD8⁺ T cells. As cytolysis of target cells represent a key effector function of CD8⁺ T cells, we also analyzed the ability of Crtam^{+/+} and Crtam^{-/-} CD8⁺ blasts to secrete granzyme B, degranulation as measured by upregulation of CD107, upregulation of FasL, and kill peptide loaded EL4 target cells. No differences were observed in each of these cytolytic parameters or in TCR expression between Crtam^{+/+} and Crtam^{-/-} CD8⁺ blasts (FIGS. 17D-F and 20). Collectively, these data indicate that Crtam is required for optimal INFγ, TNF and IL22 transcription and secretion, but not cytolytic functions.

### Crtam Maintains a Late Phase of T Cell Polarity and Selective Cytokine Production Through Scrib

As Crtam coordinates a signaling complex consisting of Scrib, Cdc42 and PKCζ to regulate IFNγ, IL17 and IL22 production in a subset of CD4⁺ T cells, we analyzed the ability of assembly of such a complex in CD8⁺ T cells. Immunoprecipitation of Scrib from activated Crtam^{-/+} CD8⁺ T cells revealed a complex that also contained Crtam, Cdc42 and PKCζ. Co-precipitation of Crtam, Cdc42 and PKCζ with Scrib was not detectable in activated Crtam^{-/-} CD8⁺ T cells (FIG. 21A). This biochemical association was further supported by confocal microscopic studies of activated CD8⁺ T cells. Crtam co-localized with Scrib, PKCζ and Talin in CD8⁺ T cells 16 hours following activation by OVA/APCs (FIG. 21B, panels 7-9, 13-15, 19-21). In addition, Crtam also co-localized with CD3 and both were oriented with the microtubule organizing center face as determined by pericentrin staining (FIG. 21B, panels 1-3, FIG. 21C and FIG. 22, panels 1-3). The inability of Crtam, Cdc42 and PKCζ to co-immunoprecipitate with Scrib in activated Crtam^{-/-} CD8⁺ T cells was also supported by the lack of Scrib, PKCζ and CD3 polarization as well as the random distribution of pericentrin 16 hours following receptor activation (FIG. 21B, panels 4-6, 10-12, 16-18, 22-24, FIG. 21C and FIG. 22, panels 4-6). Similar data were obtained with CD8⁺ T cells activated by plate-bound anti-CD3/28 mAbs (FIGS. 22A-B). In contrast to these differences observed 14+ hours post-activation, the early phase of T cell polarization of CD3 and PKCθ as well as MTOC re-orientation in Crtam^{-/-} CD8⁺ T cells were not perturbed 30 minutes following TCR engagement (FIG. 23A). In addition, absence of Crtam did not affect upregulation of early activation antigens-CD25 and CD69 (FIG. 23B).

The ability of Crtam to selectively upregulate IFNγ, TNFα and IL22 as well as maintain T cell polarity required its ability to interact with Scrib. Expression of a mutant Crtam [designated as Crtam(ΔESIV) that lacks the C-terminal PDZ-binding ESIV motif and unable to interact with Scrib] was unable to restore selective IFNγ, TNFα and IL22 production and also was unable to maintain T cell polarity in Crtam^{-/-} CD8⁻ T cells (FIGS. 21D-E).

### Scrib and PKCζ are Required to Maintain the Late Phase of T Cell Polarity That, in Turn, is Required for IFNγ, TNFα and IL22 Regulation

Selective cytokine regulation and maintenance of cellular polarity also required the presence of Scrib. Knockdown of Scrib protein in CD8⁺ T cells by Scrib siRNA resulted in a significant reduction in Scrib expression (FIG. 24A). While Crtam, Scrib and Talin were co-localized in activated CD8⁺ T cells transfected with control siRNAs (FIG. 24B, panels 1 and 3, FIG. 24C, panel 1 and FIG. 25A), Talin, Crtam and Scrib were diffusely distributed in cells transfected with Scrib siRNA (FIG. 24B, panels 2 and 4, FIG. 24C, panel 2 and FIG. 25A). TCR-activated CD8⁺ T cells, in which Scrib expression was decreased, also selectively produced less IFNγ, TNFα and IL22, without affecting IL2 levels (FIG. 24D).

A similar requirement was also observed for PKCζ. Knockdown of PKCζ protein in CD8⁺ T cells by PKCζ siRNA resulted in a significant reduction in PKCζ expression (FIG. 24E) that was associated with loss of co-localization of Crtam, Scrib and Talin

(FIG. 24B, panel 3, FIG. 24F and FIG. 25B) as well as selectively decreased production of IFNγ, TNF and IL22 (FIG. 24G). Conversely, knockdown of either Scrib or PKCζ had no effect on early T cell polarity as detected by polarization of CD3 and PKCθ 30 minutes following TCR activation and upregulation of CD25 or CD69 (FIGS. 26A and B). Hence, each of the specific signaling proteins involved in the Crtam complex-Scrib and PKCζ are functionally required for maintaining this late phase of T cell polarity and selective cytokine production.

To link Scrib and cellular polarity to selective cytokine production, we expressed a chimeric receptor whose cytoplasmic domain consists solely of Scrib in Crtam^{-/-} CD8⁺ T cells (FIG. 24H and FIG. 27). Expression of Crtam(DICD):Scrib in Crtam^{-/-} CD8⁺ T cells resulted in polarization and co-localization of Talin with the chimeric receptor and also selectively augmented IFNγ, TNFα and IL22 production (FIGS. 24I-J). Hence, a direct link exists between maintenance of cellular polarity with CD8⁺ T cell cytokine production.

Crtam Mediated CD8 T Cell Responses is Necessary for Host Resistance During L. monocytogenes Infection

Since Crtam is expressed on multiple cell lineages (including a subset of CD4⁺ T, natural killer and NKT cells) and to determine the biological importance of maintaining cellular polarity by Crtam in CD8⁻ T cells, rag2^{-/-} mice were adoptively transferred with either Crtam^{+/+} or Crtam^{-/-} CD8⁺ T cells and analyzed for immunity against the intracellular Gram-positive bacterium, Listeria monocytogenes. All mice populated with Crtam^{+/-} CD8⁺ T cells survived acute infection with 2.5×10⁵ CFU of L. monocytogenes (ATCC strain 43251) (FIGS. 28A and 29). In contrast, 80% of mice populated with Crtam^{-/-} CD8⁻ T cells succumbed to L. monocytogenes infection despite T cell repopulation. While spleens from infected mice reconstituted with Crtam^{+/+} CD8⁺ T cells were enlarged when compared to non-infected mice reconstituted with Crtam^{+/+} T cells, spleens from infected mice reconstituted with Crtam^{-/-} CD8+ T cells demonstrated multiple foci of necrosis (FIG. 28B). Correspondingly, bacteria burden of spleens reconstituted with Crtam^{-/-} CD8⁺ T cells were ˜10¹⁰ higher than spleens isolated from mice reconstituted with Crtam^{-/+} CD8+ T cells (FIG. 28C). The degree of reconstitution of Crtam^{+/-} and Crtam^{-/-} CD8⁻ T cells was comparable in both blood and spleen of adoptively transferred mice (FIGS. 29A and B). Serum IFNγ and TNFα levels from mice reconstituted with Crtam^{-/-} CD8⁺ T cells were substantially less than mice reconstituted with Crtam^{+/+} CD8⁺ T cells and similar to levels of infected rag2^{-/-} mice (FIG. 28D). Recall responses of splenocytes isolated from infected Crtam^{-/-} CD8⁺ T cell reconstituted mice also demonstrated lower levels of INFγ, TNFα and IL22, but not IL2, secretion than mice reconstituted with Crtam^{+/+} CD8+ T cells (FIG. 28E). Finally, consistent with the normal in vitro CTL activity of Crtam^{-/-} CD8⁺ T cell blasts, Crtam^{-/-} CD8⁻ T cell isolated from day 5 infected mice also demonstrated normal granzyme B secretion against L. monocytogenes infected IC-21 target cells (FIG. 28F). Hence, Crtam is required for protective CD8⁺ T cell immunity against L. monocytogenes.

### Discussion

Reorganization of the T cell cytoskeleton during the first hours following cellular activation is important for establishment of cellular polarity, MTOC reorganization, efficient and sustained generation of second messengers and subsequent T cell effector functions. While the initial phase of cytoskeletal reorganization involves recruitment of PKCθ, Vav1/Vav, Was/Wasp, Wasf2/WAVE2 and Hcls1/HS1 to the IS within the first hour of T cell activation (Fischer et al., 1998; Gomez et al., 2006; Holsinger et al., 1998; Monks et al., 1997; Nolz et al., 2006; Zhang et al., 1999; Zipfel et al., 2006), our studies here reveal an additional latter phase of cellular polarization important in a subset of CD4⁺ T cells involving Crtam, Scrib, PKCζ and Cdc42 beginning ˜6 hours following T cell activation. Our studies further show that Crtam binds Scrib via a C-terminal motif that, in turn, organizes a complex involving PKCζ and Cdc42. The Crtam-Scrib interaction is required to assemble this molecule complex and to maintain T cell polarity beyond the early phase of T cell activation. Crtam⁻ and crtam^{-/-} T cells establish normal initial phases of cytoskeletal reorganization with formation of T-cell:APC conjugates, F-actin formation, MTOC reorganization, as well as segregation and polarization of CD3, Talin, CD44 and PKCθ indistinguishable from Crtam⁺ or Crtam^{+/+} T cells. However, in the absence of Crtam-Scrib interactions, T cells are unable to sustain CD3, Talin and CD44 polarization and segregation >6 h following the initiation of T cell activation. Hence, divergent signaling machinery is involved in initiating and maintaining T cell polarity.

Prolonged maintenance of T cell polarity through Crtam is tightly associated with normal control of cellular proliferation and TCR-mediated production of IFNγ and IL22, and to a lesser degree IL17, but not IL4 or IL2. Sorted Crtam⁻ T cells or T cells expressing Crtam mutants unable to establish late T cell polarity demonstrate increased proliferative rates and secrete less IFNγ and IL22 than Crtam⁻ T cells. The selective induction by Crtam on IFNγ and IL22 appears, at minimum, to involve transcriptional activation of these genes as mRNAs of IFNγ and IL22 are augmented in Crtam⁺ and Crtam^{+/-} cells when compared to Crtam⁻ and Crtam^{-/-} T cells, respectively. Additional effects by Crtam on post-transcriptional, post-translational or on secretary pathways cannot be excluded, as have been described for regulation of IL4 and IL2 (Huse et al., 2006; Mohrs et al., 2005), though we have observed no effects of Crtam on the secretion of cytokines including IL4 and IL2.

Crtam directly interacts with Scrib, a member of the Scribble family of proteins (Scribble, Dlg and Lgl) that serve as scaffolding proteins to regulate protein-protein interactions (Humbert et al., 2006). In Drosophila and epithelial cells, loss of these genes result in mislocalization of apical proteins and adherens junctions, enhanced cellular proliferation, reduced differentiation capacity and lower thresholds to cellular transformation. Structure-function analysis of Scrib in Drosophila have demonstrated a critical role of the LRR domain in localizing Scrib to the plasma membrane, while its PDZ domains mediate interactions with yet-to-be identified proteins at septate junctions (Zeitler et al., 2004). Moreover, control of epithelial polarity and cellular proliferation, through mutational analysis of Scrib, appears to be tightly linked. Our studies demonstrate a similar regulatory pathway for late T cell polarity, control of cellular proliferation, and IFNγ and IL22 production through Crtam and Scrib. Mutation of the Scrib interacting site at the C-terminus of Crtam has a selective effect on IFNγ and IL22 production, without any overt effects on IL4. Additionally, knockdown of Scrib using siRNAs results in an inability of T cells to polarize Talin 8 h following TCR activation and decreased IFNγ and IL22, but not IL4, production. Finally, the importance of the Crtam-Scrib interaction is underscored by the ability of a Crtam(ΔICD):Scrib chimera to restore Talin polarization and to selectively augment IFNγ and IL22 production. Hence, the functional effects of Crtam on maintaining the late phase of T cell polarity and selective cytokine production depend critically upon its association with Scrib. The ability of the Scrib complex to interact with a variety of effector proteins, including the Rho GTPase regulatory βPIX-GIT complex (Audebert et al., 2004), may enable Crtam to activate and/or stabilize downstream signaling pathways, including Cdc42 and PKCζ, and contribute selectively to IFNγ and IL22, but not IL4, production. Interestingly, establishment of T cell polarity has also been recently linked during asymmetric T cell division to the generation of disparate memory and effector T cell fates (Chang et al., 2007). Absence of Crtam does not appear to impact this latter process as the numbers of effector/memory T cells are not compromised in Crtam^{-/-} mice.

In contrast to requirement of the Crtam-Scrib interaction for maintenance of late phase T cell polarity, control of cellular division, and IFNγ/IL22 production, the ECD of Crtam, while dispensable for polarity and proliferation, contributes to the regulation of IFNγ/IL22 production. Engagement of Crtam's ligand, Cadm1, augments TCR-mediated IFNγ/IL22 production in Crtam^{+/+}, but not Crtam^{-/-}, T cells. As Cadm1, the ligand for Crtam, is expressed on a subset of mouse DCs within T cell zones as well as on epithelial cells (Galibert et al., 2005; Shingai et al., 2003), the contributions of Cadm1 binding to Crtam within these microenvironments likely influences the functional programs of Crtam⁻ T cells. Cadm1 expressing cells augment IL22 mRNA expression of CD8⁺ T cells and natural killer cell responses (Boles et al., 2005; Galibert et al., 2005). As Crtam preferentially induces IFNγ and IL22, Cadm1:Crtam interactions on CD4⁺ T cells may influence T_{H}1 and T_{H}17 biology.

While our studies do not presently demonstrate a role for Crtam in T lineage commitment, linkage between cytoskeletal reorganization and T cell differentiation has been suggested by several observations. Co-localization of IFNγR with the TCR within the IS is associated with T_{H}1 lineage commitment and is inhibited by T_{H}2 differentiation (Maldonado et al., 2004). In humans, T cells deficient in the WAS protein not only exhibit defective cytoskeletal reorganization, but also demonstrate selective defects in TCR-mediated IFNγ and IL2, but not IL4, IL5 or IL10, transcriptional activation (Trifari et al., 2006). In addition to subcellular cytoskeletal reorganization, the strength and duration of TCR signal also differs between T_{H}1 and T_{H}2 cells and affect T lineage commitment (Iezzi et al., 1999). T_{H}1 differentiation can be induced with shorter duration of stimuli or less co-stimulation requirements while IL4 secretion requires longer TCR triggering (Holzer et al., 2003; Iezzi et al., 1999). Others have demonstrated that very low and very high antigen doses favor Th2-like cells (Hosken et al., 1995). Engagement of Crtam⁺ T cells 14 h following initiation of TCR activation with specialized cells that express or upregulate Cadm1 may provide an additional level of regulation of T cell differentiation and function.

Unlike other activation markers, such as CD69 and CD25, where the entire population of activated T cells expressed the upregulated marker, only a subset of activated (CD69 and CD25) naïve CD4⁺ T cells express Crtam. The inability of T cells to upregulate Crtam likely reflects a stochastic event following T cell activation. While we cannot presently exclude other instructional signals that influence Crtam expression on naive CD4⁺ T cells, Crtam is similarly upregulated on a subset of mature CD4 thymocytes ex vivo with anti-CD3 and anti-CD28 crosslinking (data not shown) and argues against an instructional model once cells exit the thymus. Nonetheless, once an activated T cell acquires Crtam on its cell surface, signaling through Crtam may represent one of many mechanisms that can influence epigenetic changes in DNA and chromatin structure surrounding IFNγ and IL22 during T cell differentiation. The ability of sorted Crtam⁺ T cells to uniformly upregulate Crtam and the inability of sorted Crtam⁻ T cells to upregulate Crtam with subsequent rounds of T cell activation are consistent with the idea that Crtam may influence the plasticity of early T cell differentiation. As Necl2, the ligand for Crtam, is expressed on a subset of CD11c^{bright}CD11b⁻CD8α⁺ mouse dendritic cells (DCs) within T cell zones as well as on epithelial cells (Galibert et al., 2005; Shingai et al., 2003), the contributions of Necl2 binding to Crtam within these microenvironments may influence the differentiation programs of Crtam⁺ T cells. Necl2 expressing cells augment IL22 mRNA expression of CD8⁺ T cells and natural killer cell responses (Boles et al., 2005; Galibert et al., 2005). As Crtam preferentially induces IFNγ and IL22, Necl2:Crtam interactions on CD4⁻ T cells may influence T_{H}1 and T_{H}17 biology. Additional studies using reporter mice that mark Crtam expression will permit further dissection of Crtam's role in the differentiation, trafficking and maintenance of T_{H} subsets. Together, our data support the idea that Crtam organizes a molecular scaffold through Scrib to regulate a previously unrecognized later phase of T cell activation and demonstrates that signals delivered beyond the first few hours of T cell activation continue to influence epigenetic changes in DNA and chromatin structure to influence T cell functions. In addition, the tight association between CRTAM expression and a restricted subset of activated T cells having important biological functions linked to various pathological disorders points to CRTAM as a viable target for therapeutic intervention.

### PARTIAL LIST OF REFERENCES

Abbas, A. R., Baldwin, D., Ma, Y., Ouyang, W., Gurney, A., Martin, F., Fong, S., van Lookeren Campagne, M., Godowski, P., Williams, P. M., et al. (2005). Immune response in silico (IRIS): immune-specific genes identified from a compendium of microarray expression data. Genes Immun 6, 319-331.
Audebert, S., Navarro, C., Nourry, C., Chasserot-Golaz, S., Lecine, P., Bellaiche, Y., Dupont, J. L., Premont, R. T., Sempere, C., Strub, J. M., et al. (2004). Mammalian Scribble forms a tight complex with the betaPIX exchange factor. Curr Biol 14, 987-995.
Batten, M., Li, J., Yi, S., Kljavin, N. M., Danilenko, D. M., Lucas, S., Lee, J., de Sauvage, F. J., and Ghilardi, N. (2006). Interleukin 27 limits autoimmune encephalomyelitis by suppressing the development of interleukin 17-producing T cells. Nat Immunol 7, 929-936.
Bilder, D., Li, M., and Perrimon, N. (2000). Cooperative regulation of cell polarity and growth by Drosophila tumor suppressors. Science 289, 113-116.
Boles, K. S., Barchet, W., Diacovo, T., Cella, M., and Colonna, M. (2005). The tumor suppressor TSLC1/NECL-2 triggers NK-cell and CD8+ T-cell responses through the cell-surface receptor CRTAM. Blood 106, 779-786.
Cantrell, D. (1996). T cell antigen receptor signal transduction pathways Annu Rev Immunol 14, 259-274.
Chang, J. T., Palanivel, V. R., Kinjyo, I., Schambach, F., Intlekofer, A. M., Banerjee, A., Longworth, S. A., Vinup, K. E., Mrass, P., Oliaro, J., et al. (2007). Asymmetric T lymphocyte division in the initiation of adaptive immune responses. Science 315, 1687-1691.
Du Pasquier, L. (2004) Innate immunity in early chordates and the appearance of adaptive immunity. C R Biol 327, 591-601.
Dustin, M. L., and Cooper, J. A. (2000). The immunological synapse and the actin cytoskeleton: molecular hardware for T cell signaling. Nat Immunol 1, 23-29.
Eckmann, L. (2006). Animal models of inflammatory bowel disease: lessons from enteric infections. Ann N Y Acad Sci 1072, 28-38.
Fischer, K. D., Kong, Y. Y., Nishina, H., Tedford, K., Marengere, L. E., Kozieradzki, I., Sasaki, T., Starr, M., Chan, G., Gardener, S., et al. (1998). Vav is a regulator of cytoskeletal reorganization mediated by the T-cell receptor. Curr Biol 8, 554-562.
Friedl, P., den Boer, A. T., and Gunzer, M. (2005). Tuning immune responses: diversity and adaptation of the immunological synapse. Nat Rev Immunol 5, 532-545.
Fuchs, A., and Colonna, M. (2006). The role of NK cell recognition of nectin and nectin-like proteins in tumor immunosurveillance. Semin Cancer Biol 16, 359-366.
Gajewski, T. F., and Fitch, F. W. (1988). Anti-proliferative effect of IFN-gamma in immune regulation. I. IFN-gamma inhibits the proliferation of Th2 but not Th1 murine helper T lymphocyte clones. J Immunol 140, 4245-4252.
Galibert, L., Diemer, G. S., Liu, Z., Johnson, R. S., Smith, J. L., Walzer, T., Comeau, M. R., Rauch, C. T., Wolfson, M. F., Sorensen, R. A., et al. (2005). Nectin-like protein 2 defines a subset of T-cell zone dendritic cells and is a ligand for class-I-restricted T-cell-associated molecule. J Biol Chem 280, 21955-21964.
Gomez, T. S., McCarney, S. D., Carrizosa, E., Labno, C. M., Comiskey, E. O., Nolz, J. C., Zhu, P., Freedman, B. D., Clark, M. R., Rawlings, D. J., et al. (2006). HS1 functions as an essential actin-regulatory adaptor protein at the immune synapse. Immunity 24, 741-752.
Holsinger, L. J., Graef, I. A., Swat, W., Chi, T., Bautista, D. M., Davidson, L., Lewis, R. S., Alt, F. W., and Crabtree, G. R. (1998). Defects in actin-cap formation in Vav-deficient mice implicate an actin requirement for lymphocyte signal transduction. Curr Biol 8, 563-572.
Holzer, U., Kwok, W. W., Nepom, G. T., and Buckner, J. H. (2003). Differential antigen sensitivity and costimulatory requirements in human Th1 and Th2 antigen-specific CD4+ cells with similar TCR avidity. J Immunol 170, 1218-1223.
Hosken, N. A., Shibuya, K., Heath, A. W., Murphy, K. M., and O'Garra, A. (1995). The effect of antigen dose on CD4+ T helper cell phenotype development in a T cell receptor-alpha beta-transgenic model. J Exp Med 182, 1579-1584.
Humbert, P. O., Dow, L. E., and Russell, S. M. (2006). The Scribble and Par complexes in polarity and migration: friends or foes? Trends Cell Biol 16, 622-630.
Huppa, J. B., and Davis, M. M. (2003). T-cell-antigen recognition and the immunological synapse. Nat Rev Immunol 3, 973-983.
Huse, M., Lillemeier, B. F., Kuhns, M. S., Chen, D. S., and Davis, M. M. (2006). T cells use two directionally distinct pathways for cytokine secretion. Nat Immunol 7, 247-255.
Iezzi, G., Scotet, E., Scheidegger, D., and Lanzavecchia, A. (1999). The interplay between the duration of TCR and cytokine signaling determines T cell polarization. Eur J Immunol 29, 4092-4101.
Kennedy, J., Vicari, A. P., Saylor, V., Zurawski, S. M., Copeland, N. G., Gilbert, D. J., Jenkins, N. A., and Zlotnik, A. (2000). A molecular analysis of NKT cells: identification of a class-I restricted T cell-associated molecule (CRTAM). J Leukoc Biol 67, 725-734.
Krummel, M. F., and Macara, I. (2006). Maintenance and modulation of T cell polarity. Nat Immunol 7, 1143-1149.
Lee, G. R., Kim, S. T., Spilianakis, C. G., Fields, P. E., and Flavell, R. A. (2006). T helper cell differentiation: regulation by cis elements and epigenetics. Immunity 24, 369-379.
Ludford-Menting, M. J., Oliaro, J., Sacirbegovic, F., Cheah, E. T., Pedersen, N., Thomas, S. J., Pasam, A., Iazzolino, R., Dow, L. E., Waterhouse, N. J., et al. (2005). A network of PDZ-containing proteins regulates T cell polarity and morphology during migration and immunological synapse formation. Immunity 22, 737-748.
Ma, A., Koka, R., and Burkett, P. (2006). Diverse functions of IL-2, IL-15, and IL-7 in lymphoid homeostasis. Annu Rev Immunol 24, 657-679.
Maldonado, R. A., Irvine, D. J., Schreiber, R., and Glimcher, L. H. (2004). A role for the immunological synapse in lineage commitment of CD4 lymphocytes. Nature 431, 527-532.
Mohrs, K., Wakil, A. E., Killeen, N., Locksley, R. M., and Mohrs, M. (2005). A two-step process for cytokine production revealed by IL-4 dual-reporter mice. Immunity 23, 419-429.
Monks, C. R., Kupfer, H., Tamir, I., Barlow, A., and Kupfer, A. (1997). Selective modulation of protein kinase C-theta during T-cell activation. Nature 385, 83-86.
Mundy, R., MacDonald, T. T., Dougan, G., Frankel, G., and Wiles, S. (2005). Citrobacter rodentium of mice and man. Cell Microbiol 7, 1697-1706.
Nagasaka, K., Nakagawa, S., Yano, T., Takizawa, S., Matsumoto, Y., Tsuruga, T., Nakagawa, K., Minaguchi, T., Oda, K., Hiraike-Wada, O., et al. (2006). Human homolog of Drosophila tumor suppressor Scribble negatively regulates cell-cycle progression from G1 to S phase by localizing at the basolateral membrane in epithelial cells. Cancer Sci.
Nolz, J. C., Gomez, T. S., Zhu, P., Li, S., Medeiros, R. B., Shimizu, Y., Burkhardt, J. K., Freedman, B. D., and Billadeau, D. D. (2006). The WAVE2 complex regulates actin cytoskeletal reorganization and CRAC-mediated calcium entry during T cell activation. Curr Biol 16, 24-34.
Osmani, N., Vitale, N., Borg, J. P., and Etienne-Manneville, S. (2006). Scrib controls Cdc42 localization and activity to promote cell polarization during astrocyte migration. Curr Biol 16, 2395-2405.
Pear, W. S., Nolan, G. P., Scott, M. L., and Baltimore, D. (1993). Production of high-titer helper-free retroviruses by transient transfection. Proc Natl Acad Sci USA 90, 8392-8396.
Round, J. L., Humphries, L. A., Tomassian, T., Mittelstadt, P., Zhang, M., and Miceli, M. C. (2007). Scaffold protein Dlghl coordinates alternative p38 kinase activation, directing T cell receptor signals toward NFAT but not NF-kappaB transcription factors. Nat Immunol 8, 154-161.
Round, J. L., Tomassian, T., Zhang, M., Patel, V., Schoenberger, S. P., and Miceli, M. C. (2005). Dlgh1 coordinates actin polymerization, synaptic T cell receptor and lipid raft aggregation, and effector function in T cells. J Exp Med 201, 419-430.
Shingai, T., Ikeda, W., Kakunaga, S., Morimoto, K., Takekuni, K., Itoh, S., Satoh, K., Takeuchi, M., Imai, T., Monden, M., and Takai, Y. (2003). Implications of nectin-like molecule-2/IGSF4/RA175/SgIGSF/TSLC1/SynCAM1 in cell-cell adhesion and transmembrane protein localization in epithelial cells. J Biol Chem 278, 35421-35427.
Tato, C. M., Laurence, A., and O'Shea, J. J. (2006). Helper T cell differentiation enters a new era: le roi est mort; vive le roi! J Exp Med 203, 809-812.
Teft, W. A., Kirchhof, M. G., and Madrenas, J. (2006). A molecular perspective of CTLA-4 function. Annu Rev Immunol 24, 65-97.
Tonikian, R., Zhang, Y., Sazinsky, S. L., Currell, B., Yeh, J.-H., Reva, B., Held, H. A., Appleton, B. A., Evangelista, M., Wu, Y., Deshayes, K., Chan, A. C., Seshagiri, S., Sander, C., Lasky, L. A., Boone, C., Bader, G. D., and Sidhu, S. S. (2007). A specificity map for the PDZ domain family. (Cell in submission).
Trifari, S., Sitia, G., Aiuti, A., Scaramuzza, S., Marangoni, F., Guidotti, L. G., Martino, S., Saracco, P., Notarangelo, L. D., Roncarolo, M. G., and Dupre, L. (2006). Defective Th1 cytokine gene transcription in CD4+ and CD8+ T cells from Wiskott-Aldrich syndrome patients. J Immunol 177, 7451-7461.
Vicente-Manzanares, M., and Sanchez-Madrid, F. (2004). Role of the cytoskeleton during leukocyte responses. Nat Rev Immunol 4, 110-122.
Xavier, R., Rabizadeh, S., Ishiguro, K., Andre, N., Ortiz, J. B., Wachtel, H., Morris, D. G., Lopez-Ilasaca, M., Shaw, A. C., Swat, W., and Seed, B. (2004). Discs large (Dlg1) complexes in lymphocyte activation. J Cell Biol 166, 173-178.
Zeitler, J., Hsu, C. P., Dionne, H., and Bilder, D. (2004). Domains controlling cell polarity and proliferation in the Drosophila tumor suppressor Scribble. J Cell Biol 167, 1137-1146.
Zhang, J., Shehabeldin, A., da Cruz, L. A., Butler, J., Somani, A. K., McGavin, M., Kozieradzki, I., dos Santos, A. O., Nagy, A., Grinstein, S., et al. (1999). Antigen receptor-induced activation and cytoskeletal rearrangement are impaired in Wiskott-Aldrich syndrome protein-deficient lymphocytes. J Exp Med 190, 1329-1342.
Zhang, Y., Yeh, S., Appleton, B. A., Held, H. A., Kausalya, P. J., Phua, D. C., Wong, W. L., Lasky, L. A., Wiesmann, C., Hunziker, W., and Sidhu, S. S. (2006). Convergent and divergent ligand specificity among PDZ domains of the LAP and zonula occludens (ZO) families. J Biol Chem 281, 22299-22311.
Zheng, Y., Danilenko, D. M., Valdez, P., Kasman, I., Eastham-Anderson, J., Wu, J., and Ouyang, W. (2007). Interleukin-22, a T(H)17 cytokine, mediates IL-23-induced dermal inflammation and acanthosis. Nature 445, 648-651.
Zipfel, P. A., Bunnell, S. C., Witherow, D. S., Gu, J. J., Chislock, E. M., Ring, C., and Pendergast, A. M. (2006). Role for the Abi/wave protein complex in T cell receptor-mediated proliferation and cytoskeletal remodeling. Curr Biol 16, 35-46.

1. A method for the treatment of an autoimmune disease, comprising administering to a subject an effective amount of a CRTAM modulator, wherein said modulator inhibits CRTAM activity in an activated T cell subpopulation, whereby the disease is treated.

2. The method of claim 1 wherein said subject is a human patient.

3. The method of claim 2 wherein said modulator comprises a monoclonal antibody.

4. The method of claim 3 wherein said antibody is a blocking, non-blocking or depleting antibody.

5. The method of claim 4 wherein said antibody is a depleting antibody.

6. The method of claim 4 wherein said antibody is an antibody fragment.

7. The method of claim 6 wherein said antibody fragment is selected from the group consisting of Fab, Fab′, F(ab′)₂, scFv, (scFv)₂, dAb, complementarity determining region (CDR) fragments, linear antibodies, single-chain antibody molecules, minibodies, diabodies, and multispecific antibodies formed from antibody fragments.

8. The method of claim 4 wherein said antibody is chimeric, humanized or human.

9. The method of claim 5 wherein said depleting antibody is an antibody conjugate comprising a toxin.

10. The method of claim 9, wherein said toxin is a cytotoxic agent selected from the group consisting of a radioactive isotope, an enzyme, and a small molecule toxin.

11. The method of claim 2, wherein said autoimmune disease is selected from the group consisting of rheumatoid arthritis (RA); multiple sclerosis (MS); systemic lupus erythematosus (SLE); lupus nephritis; cutaneous lupus erythematosus (CLE); autoimmune hepatitis; juvenile rheumatoid arthritis; infectious hepatitis; primary biliary cirrhosis; psoriasis; dermatitis; atopic dermatitis; systemic scleroderma; systemic sclerosis; Crohn's disease, ulcerative colitis; respiratory distress syndrome; adult respiratory distress syndrome; ARDS; meningitis; encephalitis; uveitis; glomerulonephritis; pemphigus; macrophage activation syndrome; eczema; asthma; atherosclerosis; leukocyte adhesion deficiency; diabetes mellitus; Type I diabetes mellitus; insulin dependent diabetes mellitis; allergic rhinitis; autoimmune reaction associated with organ transplantation; Reynaud's syndrome; autoimmune thyroiditis; Hashimoto's thyroiditis; allergic encephalomyelitis; Sjogren's syndrome; juvenile onset diabetes; immune responses associated with acute and delayed hypersensitivity mediated by cytokines and T-lymphocytes; tuberculosis, sarcoidosis, polymyositis, granulomatosis; vasculitis; pernicious anemia (Addison's disease); diseases involving leukocyte diapedesis; central nervous system (CNS) inflammatory disorder; multiple organ injury syndrome; hemolytic anemia; cryoglobinemia; Coombs positive anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) and autoimmune thrombocytopenia.

12. The method of claim 11 wherein said autoimmune diseases is selected from the group consisting of rheumatoid arthritis (RA), multiple sclerosis (MS), systemic lupus erythematosus (SLE), cutaneous lupus erythematosus (CLE), psoriasis, Crohn's disease, ulcerative colitis, uveitis, atopic dermatitis, asthma, autoimmune reaction associated with organ transplantation, autoimmune hepatitis, juvenile rheumatoid arthritis, infectious hepatitis, glomerulonephritis, primary biliary cirrhosis, vasculitis, pemphigus, macrophage activation syndrome, allergic rhinitis, diabetes mellitus 1, and diabetes mellitus 2.

13. The method of claim 1, wherein said autoimmune disease is characterized by the presence of activated CD4⁺CRTAM⁺ T cells.

14. The method of claim 13, wherein said T cells are further characterized by elevated levels of expression of one or more cytokines when compared to expression of said one or more cytokines in CD4⁻CRTAM⁻ T cells.

15. The method of claim 14, wherein said T cells are further characterized by elevated secretion levels of one or more cytokines when compared to secretion levels of said one or more cytokines in CD4⁻CRTAM⁻ T cells.

16. The method of claim 14, wherein said cytokine is IFNγ.

17. The method of claim 14, wherein said cytokine is IL22.

18. The method of claim 14, wherein said cytokine is IFNγ, IL22 or IL17.

19. The method of 13, wherein said T cells are autoreactive.

20. The method of claim 1, wherein said treatment is characterized by a decrease in cytokine expression in said mammalian subject as compared to a mammalian subject not administered said CRTAM modulator.

21. The method of claim 20, wherein said treatment is further characterized by a decrease in cytokine secretion levels in said subject as compared to a subject not administered said CRTAM modulator.

22. The method of claim 20, wherein said cytokine is IFNγ.

23. The method of claim 20, wherein said cytokine is IL22.

24. The method of claim 20, wherein said cytokine is IFNγ, IL22 or IL17.

25. A method of treating a disorder comprising administering to a subject an effective amount of a CRTAM modulator that enhances CD4+ T cell activity, wherein said disorder is a cancer, an immune deficiency, or an infection.

26. A method of inhibiting a biological activity associated with T cell activation comprising contacting a CRTAM modulator with a T cell, wherein said modulator inhibits activity of a CRTAM-expressing T cell.

27. The method of claim 26 wherein said modulator comprises a monoclonal antibody.

28. The method of claim 27, wherein the antibody is afucosylated.

29. The method of claim 27 wherein said antibody is a blocking, non-blocking or depleting antibody.

30. The method of claim 29 wherein said antibody is a depleting antibody.

31. The method of claim 29 wherein said antibody is an antibody fragment.

32. The method of claim 31 wherein said antibody fragment is selected from the group consisting of Fab, Fab′, F(ab′)₂, scFv, (scFv)₂, dAb, complementarity determining region (CDR) fragments, linear antibodies, single-chain antibody molecules, minibodies, diabodies, and multispecific antibodies formed from antibody fragments.

33. The method of claim 29 wherein said antibody is chimeric, humanized or human.

34. The method of claim 30 wherein said antibody is an antibody conjugate comprising a toxin.

35. The method of claim 34 wherein said toxin is a cytotoxic agent selected from the group consisting of a radioactive isotope, an enzyme, and a small molecule toxin.

36. A method of enhancing a biological activity associated with T cell activation comprising contacting a CRTAM modulator with a CD4+ T cell, wherein said modulator enhances activity of a CRTAM-expressing CD4+ T cell.

37. A method for detecting an autoimmune disease comprising:
(a) contacting a CRTAM modulator with a first test sample comprising T cells from a subject and a control; and
(b) determining that a relative amount of CRTAM⁺ cells in said test sample is higher than a relative amount of CRTAM⁺ cells in said control, wherein said higher relative amount of CRTAM⁺ cells in said test sample is indicative of an autoimmune disease in said mammalian subject.

38. The method of claim 37, further comprising
(c) obtaining a second test sample comprising T cells from said subject;
(d) contacting a CRTAM modulator with said second test sample; and
(e) determining that a relative amount of CRTAM⁺ cells in said second test sample is higher than a relative amount of CRTAM⁺ cells in said first test sample,
wherein said higher relative amount of CRTAM⁺ cells in said second test sample is indicative of a flare-up in said autoimmune disease in the subject from which said test samples were obtained.

39. The method of claim 37, wherein the test sample comprises CRTAM⁺ T cells.

40. The method of claim 37, wherein the test sample comprises CD4⁺ T cells.

41. The method of claim 37, wherein the test sample comprises CD8⁺ T cells.

42. The method of claim 37, wherein the test sample comprises CD4⁺ T cells and CD8⁺ T cells.

43. The method of claim 37, wherein said autoimmune disease is selected from the group consisting of autoimmune disease is selected from the group consisting of rheumatoid arthritis (RA); multiple sclerosis (MS); systemic lupus erythematosus (SLE); lupus nephritis; cutaneous lupus erythematosus (CLE); autoimmune hepatitis; juvenile rheumatoid arthritis, infectious hepatitis; primary biliary cirrhosis; psoriasis; dermatitis; atopic dermatitis; systemic scleroderma; systemic sclerosis; Crohn's disease, ulcerative colitis; respiratory distress syndrome; adult respiratory distress syndrome; ARDS; meningitis; encephalitis; uveitis; glomerulonephritis; pemphigus; macrophage activation syndrome; eczema; asthma; atherosclerosis; leukocyte adhesion deficiency; diabetes mellitus; Type I diabetes mellitus; insulin dependent diabetes mellitis; allergic rhinitis; autoimmune reaction associated with organ transplantation; Reynaud's syndrome; autoimmune thyroiditis; Hashimoto's thyroiditis; allergic encephalomyelitis; Sjogren's syndrome; juvenile onset diabetes; immune responses associated with acute and delayed hypersensitivity mediated by cytokines and T-lymphocytes; tuberculosis, sarcoidosis, polymyositis, granulomatosis; vasculitis; pernicious anemia (Addison's disease); diseases involving leukocyte diapedesis; central nervous system (CNS) inflammatory disorder; multiple organ injury syndrome; hemolytic anemia; cryoglobinemia; Coombs positive anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) and autoimmune thrombocytopenia.

44. The method of claim 43, wherein said autoimmune disease is selected from the group consisting of rheumatoid arthritis (RA), multiple sclerosis (MS), systemic lupus erythematosus (SLE), cutaneous lupus erythematosus (CLE), psoriasis, Crohn's disease, ulcerative colitis, uveitis, atopic dermatitis, asthma, autoimmune reaction associated with organ transplantation, autoimmune hepatitis, juvenile rheumatoid arthritis, infectious hepatitis, glomerulonephritis, primary biliary cirrhosis, vasculitis, pemphigus, macrophage activation syndrome, allergic rhinitis, diabetes mellitus 1, and diabetes mellitus 2.

45. The method of claim 37, wherein said autoimmune disease is an active autoimmune disease.

46. The method of claim 37, wherein said active autoimmune disease is characterized by the presence of activated T cells, wherein said T cells are CD4⁺CRTAM⁺ T cells.

47. The method of claim 46, wherein said activated T cells are further characterized by elevated expression levels of one or more cytokines as compared to expression levels of said one or more cytokines in CD4⁻CRTAM⁻ T cells.

48. The method of claim 47, wherein said activated T cells are further characterized by elevated secretion levels of one or more cytokines as compared to secretion levels of said one or more cyotokines in CD4⁺CRTAM⁻ T cells.

49. The method of claim 47, wherein said cytokine is IFNγ.

50. The method of claim 47, wherein said cytokine is IL22.

51. The method of claim 47, wherein said cytokine is IFNγ, IL22 or IL17.

52. The method of claim 46, wherein said activated T cells are autoreactive.

53. An isolated population of activated CD4+ T cells characterized by (1) expression of CRTAM; and (2) an elevated expression level of one or more cytokines relative to CD4+ activated T cells not expressing CRTAM.

54. The population of CD4+ T cells of claim 53 wherein said cytokine is IFNγ, IL22 or IL 17.

55. A method for isolating a population of activated CD4+ T cells, comprising contacting a sample comprising a mixed population of T cells from a subject with an anti-CRTAM antibody and separating any cells from said mixed population that do not bind said CRTAM antibody, wherein a population of activated CD4+ T cells bound by said CRTAM antibody remains in said sample.

56. The method of claim 55 further comprising separating said bound CRTAM antibody from said population of activated CD4+ cells bound to said CRTAM antibody.

57. A kit comprising a CRTAM modulator and instructions for administering said modulator to treat an autoimmune disease.

58. The kit of claim 57 wherein said modulator comprises a monoclonal antibody.

59. The kit of claim 58 wherein said antibody is a blocking, non-blocking or depleting antibody.

60. The kit of claim 59 wherein said antibody is a depleting antibody.

61. The kit of claim 59 wherein said antibody is an antibody fragment.

62. The kit of claim 61 wherein said antibody fragment is selected from the group consisting of Fab, Fab′, F(ab′)₂, scFv, (scFv)₂, dAb, complementarity determining region (CDR) fragments, linear antibodies, single-chain antibody molecules, minibodies, diabodies, and multispecific antibodies formed from antibody fragments.

63. The kit of claim 59 wherein said antibody is chimeric, humanized or human.

64. The kit of claim 60 wherein said depleting antibody is an antibody conjugate comprising a toxin.

65. The kit of claim 64, wherein said toxin is a cytotoxic agent selected from the group consisting of a radioactive isotope, an enzyme, and a small molecule toxin.

66. Use of a CRTAM modulator in the preparation of a medicament to treat an autoimmune disease.

67. The use of claim 66 wherein said modulator comprises a monoclonal antibody.

68. The use of claim 67 wherein said antibody is a blocking, non-blocking or depleting antibody.

69. The use of claim 68 wherein said antibody is a depleting antibody.

70. The use of claim 68 wherein said antibody is an antibody fragment.

71. The use of claim 70 wherein said antibody fragment is selected from the group consisting of Fab, Fab′, F(ab′)₂, scFv, (scFv)₂, dAb, complementarity determining region (CDR) fragments, linear antibodies, single-chain antibody molecules, minibodies, diabodies, and multispecific antibodies formed from antibody fragments.

72. The use of claim 68 wherein said antibody is chimeric, humanized or human.

73. The use of claim 69 wherein said depleting antibody is an antibody conjugate comprising a toxin.

74. The use of claim 73, wherein said toxin is a cytotoxic agent selected from the group consisting of a radioactive isotope, an enzyme, and a small molecule toxin.

75. A CRTAM modulator for use in the treatment of an autoimmune disease.

76. The modulator of claim 75 wherein said modulator comprises a monoclonal antibody.

77. The antibody of claim 76 wherein said antibody is a blocking, non-blocking or depleting antibody.

78. The antibody of claim 77 wherein said antibody is a depleting antibody.

79. The antibody of claim 77 wherein said antibody is an antibody fragment.

80. The antibody of claim 79 wherein said antibody fragment is selected from the group consisting of Fab, Fab′, F(ab′)₂, scFv, (scFv)₂, dAb, complementarity determining region (CDR) fragments, linear antibodies, single-chain antibody molecules, minibodies, diabodies, and multispecific antibodies formed from antibody fragments.

81. The antibody of claim 77 wherein said antibody is chimeric, humanized or human.

82. The antibody of claim 78 wherein said depleting antibody is an antibody conjugate comprising a toxin.

83. The antibody of claim 82, wherein said toxin is a cytotoxic agent selected from the group consisting of a radioactive isotope, an enzyme, and a small molecule toxin.
